SEQUENCE LISTING (10 sequences)

SEQ ID NO: 1 - DNA, 1863 bp, triticum turgidum spp. Durum
  Rht-a1a Wild type allele of Rht-a1
atgaagcgcgagtaccaggacgccggcgggagcggtggcgggggcggcatgggctcgtctgaggacaagatgatggtgtcggcggcggcgggggagggggaggaggtggacgagctgctggcggcgctcgggtacaaggtgcgggcctccgacatggcggacgtggcgcagaagctggagcagctggagatggccatggggatgggcggcgtgggcgccggcgccgcccccgacgacagcttcgccacccacctcgccacggacaccgtgcactacaaccccaccgacctctcctcctgggtcgagagcatgctgtcggagctcaacgcgccgccgccgcccctcccgcccgccccgcagcagctcaacgcctccacctcctccaccgtcacgggcggtgggtacttcgatctcccgccctcggttgactcctcctgcagcacctacgccctgcggccgatcccctccccggccggcgccgtcgggccggccgacctgtccgccgactccgtgcgggaccccaagcggatgcgcactggcgggagcagcacctcgtcgtcgtcatcctcctcgtcctctctcggtgggggcgccaggagctctgtggtggaggctgctccgccggtcgcggccggggccaacgcgcccgcgctgccggtcgtcgtggtcgacacgcaggaggccgggattcggctggtgcacgcgctgctggcctgcgcggaggccgtgcagcaggagaacttctctgccgcggaggcgctggtgaagcagatacccttgctggccgcgtcccagggcggcgcgatgcgcaaggtcgccgcctacttcggcgaggccctcgcccgccgcgtcttccgcttccgcccgcagccggacagctccctcctcgacgccgccttcgccgacctcctccacgcgcacttctacgagtcctgcccctacctcaagttcgcccacttcaccgccaaccaggccatcctggaggccttcgccggctgccgccgcgtgcacgtcgtcgacttcggcatcaagcaggggatgcagtggcccgccctcctccaggccctcgcgctccgtcccggcggccctccctcgttccgcctcaccggcgtcggccccccgcagccggacgagaccgacgccttgcagcaggtgggctggaagctcgcccagttcgcgcacaccatccgcgtcgacttccagtaccgcggcctcgtcgccgccacgctcgcggacctggagccattcatgctgcagccggagggcgaggaggacccgaacgaggagcccgaggtaatcgccgtcaactcggtcttcgagatgcaccggctgctcgcgcagcccggcgccctggagaaggtcctgggcaccgtgcgcgccgtgcggccgaggatcgtcaccgtggtggagcaggaggccaaccacaactccggcacattcctggaccgcttcaccgagtctctgcactactactccaccatgttcgattccctcgagggcggcagctccggcggcccatccgaagtctcatcgggggctgccgctgctcctgccgccgccggcacggaccaggtcatgtccgaggtgtacctcggccggcagatctgcaacgtggtggcctgcgagggggcggagcgcacagagcgccacgagacgctggggcagtggcggaaccggctgggcaacgccgggttcgagaccgtgcacctgggctccaatgcctacaagcaggcgagcacgctgctggccctattcgccggcggcgacgggtacaaggtggaggagaaggagggctgcctgactctcgggtggcacacgcgcccgctgatcgccacctcggcatggcgcctggccgcgccgtga

SEQ ID NO: 2 - DNA, 1863 bp, triticum turgidum spp. Durum
  Mutant allele of Rht-a1a
atgaagcgcgagtaccaggacgccggcgggagcggtggcgggggcggcatgggctcgtctgaggacaagatgatggtgtcggcggcggcgggggagggggaggaggtggacgagctgctggcggcgctcgggtacaaggtgcgggcctccgacatggcggacgtggcgcagaagctggagcagctggagatggccatggggatgggcggcgtgggcgccggcgccgcccccgacgacagcttcgccacccacctcgccacggacaccgtgcactacaaccccaccgacctctcctcctgggtcgagagcatgctgtcggagctcaacgcgccgccgccgcccctcccgcccgccccgcagcagctcaacgcctccacctcctccaccgtcacgggcggtgggtacttcgatctcccgccctcggttgactcctcctgcagcacctacgccctgcggccgatcccctccccggccggcgccgtcgggccggccgacctgtccgccgactccgtgcgggaccccaagcggatgcgcactggcgggagcagcacctcgtcgtcgtcatcctcctcgtcctctctcggtgggggcgccaggagctctgtggtggaggctgctccgccggtcgcggccggggccaacgcgcccgcgctgccggtcgtcgtggtcgacacgcaggaggccgggattcggctggtgcacgcgctgctggcctgcgcggaggccgtgcagcaggagaacttctctgccgcggaggcgctggtgaagcagatacccttgctggccgcgtcccagggcggcgcgatgcgcaaggtcgccgcctacttcggcgaggccctcgcccgccgcgtcttccgcttccgcccgcagccggacagctccctcctcgacgccgccttcgccgacctcctccacgcgcacttctacgagtcctgcccctacctcaagttcgcccacttcaccgccaaccaggccatcctggaggccttcgccggctgccgccgcgtgcacgtcgtcgacttcggcatcaagcaggggatgcagtggcccgccctcctccaggccctcgcgttccgtcccggcggccctccctcgttccgcctcaccggcgtcggccccccgcagccggacgagaccgacgccttgcagcaggtgggctggaagctcgcccagttcgcgcacaccatccgcgtcgacttccagtaccgcggcctcgtcgccgccacgctcgcggacctggagccattcatgctgcagccggagggcgaggaggacccgaacgaggagcccgaggtaatcgccgtcaactcggtcttcgagatgcaccggctgctcgcgcagcccggcgccctggagaaggtcctgggcaccgtgcgcgccgtgcggccgaggatcgtcaccgtggtggagcaggaggccaaccacaactccggcacattcctggaccgcttcaccgagtctctgcactactactccaccatgttcgattccctcgagggcggcagctccggcggcccatccgaagtctcatcgggggctgccgctgctcctgccgccgccggcacggaccaggtcatgtccgaggtgtacctcggccggcagatctgcaacgtggtggcctgcgagggggcggagcgcacagagcgccacgagacgctggggcagtggcggaaccggctgggcaacgccgggttcgagaccgtgcacctgggctccaatgcctacaagcaggcgagcacgctgctggccctattcgccggcggcgacgggtacaaggtggaggagaaggagggctgcctgactctcgggtggcacacgcgcccgctgatcgccacctcggcatggcgcctggccgcgccgtga

SEQ ID NO: 3 - DNA, 1863 bp, triticum turgidum spp. Durum
  Mutant allele of Rht-a1a
atgaagcgcgagtaccaggacgccggcgggagcggtggcgggggcggcatgggctcgtctgaggacaagatgatggtgtcggcggcggcgggggagggggaggaggtggacgagctgctggcggcgctcgggtacaaggtgcgggccttcgacatggcggacgtggcgcagaagctggagcagctggagatggccatggggatgggcggcgtgggcgccggcgccgcccccgacgacagcttcgccacccacctcgccacggacaccgtgcactacaaccccaccgacctctcctcctgggtcgagagcatgctgtcggagctcaacgcgccgccgccgcccctcccgcccgccccgcagcagctcaacgcctccacctcctccaccgtcacgggcggtgggtacttcgatctcccgccctcggttgactcctcctgcagcacctacgccctgcggccgatcccctccccggccggcgccgtcgggccggccgacctgtccgccgactccgtgcgggaccccaagcggatgcgcactggcgggagcagcacctcgtcgtcgtcatcctcctcgtcctctctcggtgggggcgccaggagctctgtggtggaggctgctccgccggtcgcggccggggccaacgcgcccgcgctgccggtcgtcgtggtcgacacgcaggaggccgggattcggctggtgcacgcgctgctggcctgcgcggaggccgtgcagcaggagaacttctctgccgcggaggcgctggtgaagcagatacccttgctggccgcgtcccagggcggcgcgatgcgcaaggtcgccgcctacttcggcgaggccctcgcccgccgcgtcttccgcttccgcccgcagccggacagctccctcctcgacgccgccttcgccgacctcctccacgcgcacttctacgagtcctgcccctacctcaagttcgcccacttcaccgccaaccaggccatcctggaggccttcgccggctgccgccgcgtgcacgtcgtcgacttcggcatcaagcaggggatgcagtggcccgccctcctccaggccctcgcgctccgtcccggcggccctccctcgttccgcctcaccggcgtcggccccccgcagccggacgagaccgacgccttgcagcaggtgggctggaagctcgcccagttcgcgcacaccatccgcgtcgacttccagtaccgcggcctcgtcgccgccacgctcgcggacctggagccattcatgctgcagccggagggcgaggaggacccgaacgaggagcccgaggtaatcgccgtcaactcggtcttcgagatgcaccggctgctcgcgcagcccggcgccctggagaaggtcctgggcaccgtgcgcgccgtgcggccgaggatcgtcaccgtggtggagcaggaggccaaccacaactccggcacattcctggaccgcttcaccgagtctctgcactactactccaccatgttcgattccctcgagggcggcagctccggcggcccatccgaagtctcatcgggggctgccgctgctcctgccgccgccggcacggaccaggtcatgtccgaggtgtacctcggccggcagatctgcaacgtggtggcctgcgagggggcggagcgcacagagcgccacgagacgctggggcagtggcggaaccggctgggcaacgccgggttcgagaccgtgcacctgggctccaatgcctacaagcaggcgagcacgctgctggccctattcgccggcggcgacgggtacaaggtggaggagaaggagggctgcctgactctcgggtggcacacgcgcccgctgatcgccacctcggcatggcgcctggccgcgccgtga

SEQ ID NO: 4 - DNA, 1864 bp, triticum turgidum spp. Durum
  Rht-B1a Wild type allele of Rht-B1
atgaagcgcgagtaccaggacgccggagggagcggcggtggcgggggaggcatgggctcgtccgaggacaagatgatggtgtcggggtcggcggcggcgggggagggggaggaggtggacgagctgctggcggcgctcgggtacaaggtgcgggcgtccgacatggcggacgtggcgcagaagctggagcagctggagatggccatggggatgggcggcgtgggcgccggcgccgcgcccgacgacagcttcgccacccacctcgccacggacaccgtgcactacaaccccaccgacctctcctcctgggtcgagagcatgctgtcggagctcaacgcgccgccgccgcccctcccgcccgccccgcagctcaacgcctccacctcctccaccgtcaccggcggcgggtacttcgatctcccgccctccgtcgactcctcctgcagcacctacgcgctgcggccgatcccgtccccggccgtcgcgccggccgacctctccgccgactccgtcgtgcgggatcccaagcggatgcgcactggcggcagcagcacctcgtcgtcatcctcatcgtcctctctcggcggtggcggcgccaggagctctgtggtggaggctgccccgccggtggccgccgcggccggtgcgcccgcgctgccggtcgtcgtggtcgacacgcaggaggccgggattcggctggtgcacgcgctgctggcgtgcgcagaggccgtgcagcaggagaacttctctgccgcggaggcgctggtgaagcagatacccttgctggccgcgtcccagggcggcgccatgcgcaaggtcgccgcctacttcggcgaggccctcgcccgccgcgtcttccgcttccgcccgcagccggacagctccctcctcgacgccgccttcgccgacctcctccacgcgcacttctacgagtcctgcccctacctcaagttcgcccacttcaccgccaaccaggccatcctggaggcgttcgccggctgccgccgcgtgcacgtcgtcgacttcggcatcaagcaggggatgcagtggcccgcccttctccaggccctggcgctccgtcccggcggccctccctcgttccgcctcaccggcgtcggccccccgcagccggacgagaccgacgccttgcagcaggtgggctggaagctcgcccagttcgcgcacaccatccgcgtcgacttccagtaccgcggcctcgtcgccgccacgctcgcggacctggagccgttcatgctgcagccggagggcgaggaggacccgaacgaggagcccgaggtaatcgccgtcaactcggtcttcgagatgcaccggctgctcgcgcagcccggcgccctggagaaggtcctgggcaccgtgcgcgccgtgcggccgaggatcgtcaccgtggtggagcaggaggcgaaccacaactccggcacattcctggaccgcttcaccgagtccctgcactactactccaccatgttcgattctctggagggcggcagctccggcggcccatccgaagtctcatctggggcggctgctgctcctgccgccgccggcacggaccaggtcatgtccgaggtgtacctcggccggcagatctgcaacgtggtggcctgcgagggggcggagcgcacagagcggcacgagaccctggggcagtggcggaaccgcctcggcaacgccgggttcgagaccgtccacctgggctccaatgcctacaagcaggcgagcacgctgctggcgctcttcgcaggcggcgacgggtacaaggtggaggagaaggagggctgcctgacgctggggtggcacacgcgcccgctgatcgccacctccgcatggcgcctggccgcgccga

SEQ ID NO: 5 - DNA, 1864 bp, triticum turgidum spp. Durum
  Mutant allele of Rht-B1b
atgaagcgcgagtaccaggacgccggagggagcggcggtggcgggggaggcatgggctcgtccgaggacaagatgatggtgtcggggtcggcggcggcgggggagggggaggaggtggacgagctgctggcggcgctcgggtacaaggtgcgggcgtccgacatggcggacgtggcgcagaagctggagtagctggagatggccatggggatgggcggcgtgggcgccggcgccgcgcccgacgacagcttcgccacccacctcgccacggacaccgtgcactacaaccccaccgacctctcctcctgggtcgagagcatgctgtcggagctcaacgcgccgccgccgcccctcccgcccgccccgcagctcaacgcctccacctcctccaccgtcaccggcggcgggtacttcgatctcccgccctccgtcgactcctcctgcagcacctacgcgctgcggccgatcccgtccccggccgtcgcgccggccgacctctccgccgactccgtcgtgcgggatcccaagcggatgcgcactggcggcagcagcacctcgtcgtcatcctcatcgtcctctctcggcggtggcggcgccaggagctctgtggtggaggctgccccgccggtggccgccgcggccggtgcgcccgcgctgccggtcgtcgtggtcgacacgcaggaggccgggattcggctggtgcacgcgctgctggcgtgcgcagaggccgtgcagcaggagaacttctctgccgcggaggcgctggtgaagcagatacccttgctggccgcgtcccagggcggcgccatgcgcaaggtcgccgcctacttcggcgaggccctcgcccgccgcgtcttccgcttccgcccgcagccggacagctccctcctcgacgccgccttcgccgacctcctccacgcgcacttctacgagtcctgcccctacctcaagttcgcccacttcaccgccaaccaggccatcctggaggcgttcgccggctgccgccgcgtgcacgtcgtcgacttcggcatcaagcaggggatgcagtggcccgcccttctccaggccctggcgctccgtcccggcggccctccctcgttccgcctcaccggcgtcggccccccgcagccggacgagaccgacgccttgcagcaggtgggctggaagctcgcccagttcgcgcacaccatccgcgtcgacttccagtaccgcggcctcgtcgccgccacgctcgcggacctggagccgttcatgctgcagccggagggcgaggaggacccgaacgaggagcccgaggtaatcgccgtcaactcggtcttcgagatgcaccggctgctcgcgcagcccggcgccctggagaaggtcctgggcaccgtgcgcgccgtgcggccgaggatcgtcaccgtggtggagcaggaggcgaaccacaactccggcacattcctggaccgcttcaccgagtccctgcactactactccaccatgttcgattctctggagggcggcagctccggcggcccatccgaagtctcatctggggcggctgctgctcctgccgccgccggcacggaccaggtcatgtccaaggtgtacctcggccggcagatctgcaacgtggtggcctgcgagggggcggagcgcacagagcggcacgagaccctggggcagtggcggaaccgcctcggcaacgccgggttcgagaccgtccacctgggctccaatgcctacaagcaggcgagcacgctgctggcgctcttcgcaggcggcgacgggtacaaggtggaggagaaggagggctgcctgacgctggggtggcacacgcgcccgctgatcgccacctccgcatggcgcctggccgcgccga

SEQ ID NO: 6 - DNA, 1059 bp, triticum turgidum spp. Durum
  tatB1-4a Wild type allele of tatB1-4a
atgtttcctttctatgattccccgagccccatggacttacccctttaccaacagctgcagatcagccctccctcgccaaaggcgccggatcaccaatccttgctctactaccattcctcccctgcattcgccgccgacgccttccaccacagctacctctgtgccggcgccgcgacgccgcccgcggccgagatcgacaaccagccgccaccggagctgctgctgatggatcaggcgccagcgccaagggcagacggtgttggaaccgcacaaggcctgcacggcggtggaggcctcgacagcgcggcggctgccgcggccaggaaagaccggcacagcaagatatgcaccgccggcgggatgagggaccggcggatgcggctgtccctcgacgtcgcccgcaagttcttcgcgctccaggacatgctcggcttcgacaaggccagcaagacggtgcaatggctcctcaatacgtcaaagggcgccatcagggaggtcatgactgacgaggcgtcctccgactgcgaggaggacggctccagcagcctctccgtcgtcgacggcaagcacaagccgccagggacggaggctggaggtggtgatcacggcgaggggaagaagccgatgccaagggcagctagaagagcacccgccaatccaaagccgcaaaggaaattggccagtgcgcacccgatccccgacaaggagtcgaggacgaaggcgcgggagagggccagggagcggacgcgggaaaagaaccggatgcgatgggttacgctcgcgtctacgattaacatcgagccggcagccaccggcatggcggcggcgagggcggatgagttgatcacgaggccgcacaattttatcaatcgctcctcgtccatgaacacagcaagcgctgaattggaggaggggtgctcgtcgtccatgccgagcgaagctatcgtggctggcttcggcaatggagggtacggaagcagcggcaactactaccagtatcagctggagcagcaatgggagctcgctggagtggtgtttcccaactcgcagccctactga

SEQ ID NO: 7 - DNA, 1059 bp, triticum turgidum spp. Durum
  tatB1-4a Mutant allele of tatB1-4a
atgtttcctttctatgattccccgagccccatggacttacccctttaccaacagctgcagatcagccctccctcgccaaaggcgccggatcaccaatccttgctctactaccattcctcccctgcattcgccgccgacgccttccaccacagctacctctgtgccggcgccgcgacgccgcccgcggccgagatcgacaaccagccgccaccggagctgctgctgatggatcaggcgccagcgccaagggcagacggtgttggaaccgcacaaggcctgcacggcggtggaggcctcgacagcgcggcggctgccgcggccaggaaagaccggcacagcaagatatgcaccgccggcgggatgagggaccggcggatgcggctgtccctcgacgtcgcccgcaagttcttcgcgctccaggacatgctcggcttcgacaaggccagcaagacggtgcaatggctcctcaatacgtcaaagggcgccatcagggaggtcatgactgacgaggcgtcctccgactgcgaggaggacggctccagcagcctctccgtcgtcgacggcaagcacaagccgccagggacggaggctggaggtggtgatcacggcgaggggaagaagccgatgccaagggcagctagaagagcacccgccaatccaaagccgcaaaggaaattggccagtgcgcacccgatccccgacaaggagtcgaggacgaaggcgcgggagagggccagggagcggacgcgggaaaagaaccggatgcgatgggttacgctcgcgtctacgattaacatcgagccggcagccaccggcatggcggcggcgagggcggatgagttgatcacgaggccgcacaattttatcaatcgctcctcgtccatgaacacagcaagcgctgaattggaggaggggtgctcgtcgtccatgccgagcgaagctatcgtggctggcttcggcaatggagggtacggaagcagcggcaactactaccagtatcagctggagcagcaataggagctcgctggagtggtgtttcccaactcgcagccctactga

SEQ ID NO: 8 - DNA, 1065 bp, triticum turgidum spp. Durum
  tatB1-4B Wild type allele of tatB1-4B
atgtttcctttctatgattcccctagccccatggacttgcccctttaccaacagctgcagatcagccctccctcgccaaaggcgccggatcaccaatccttgctctactaccattcctcccctgcatttgccgccgactccttccaccacagctacctctgtgccggtgccgcgacgcccacgccgcccgcggccgagatcgacaaccagtcgccgccggagctgctgctgatggatcaggctccggcgccaagggcagacggtgttggaaccgcacaaggcctgcacggcggtggaggcctcgacagcgcggcggccgccgcggccaggaaagaccggcacagcaagatatgcaccgccggcgggatgagggaccggcggatgcggctctccctcgacgtcgcccgcaagttcttcgcgctccaggacatgctcggcttcgacaaggccagcaagacggtgcaatggctcctcaatacgtcaaagggcgccatcagggaggtcatgactgacgaggcgtcctccgactgcgaggaggacggctccagcagcctctccgtcgtcgacggcaagcacaagccgccagggacggaggctgaaggtggtgatcacggcgaggggaagaagcaggtgccaagggcagccagaagggcacccgccaaaccaaagccgcaaaggaaattggccagtgcgcacccgatccccgacaaggagtcgaggacgaaggcgcgggagagggcgagggagcggactagggaaaagaaccggatgcgatgggtgacgctcgcgtccacaattaacatcgagccggcagccaccggcatggcggcggcgagggcggacgagttcgtcacgaggccgaacaatttgatcagtcgctcctcgtccatgaacacagcaagagctgaattggaggaggggtgctcgtcgtccatgccgagcgaagcgatcatggctggcttcggcaatggaggatacggaagcagcggtaactactaccagtatcagctggagcagcaatgggagctcggtggagtggtgtttcccaactcccagccctactga

SEQ ID NO: 9 - DNA, 1065 bp, triticum turgidum spp. Durum
  tatB1-4B Mutant allele of tatB1-4B
atgtttcctttctatgattcccctagccccatggacttgcccctttaccaacagctgcagatcagccctccctcgccaaaggcgccggatcaccaatccttgctctactaccattcctcccctgcatttgccgccgactccttccaccacagctacctctgtgccggtgccgcgacgcccacgccgcccgcggccgagatcgacaaccagtcgccgccggagctgctgctgatggatcaggctccggcgccaagggcagacggtgttggaaccgcacaaggcctgcacggcggtggaggcctcgacagcgcggcggccgccgcggccaggaaagaccggcacagcaagatatgcaccgccggcgggatgagggaccggcggatgcggctctccctcgacgtcgcccgcaagttcttcgcgctccaggacatgctcggcttcgacaaggccagcaagacggtgcaatggctcctcaatacgtcaaagggcgccatcagggaggtcatgactgacgaggcgtcctccgactgcgaggaggacggctccagcagcctctccgtcgtcgacggcaagcacaagccgccagggacggaggctgaaggtggtgatcacggcgaggggaagaagcaggtgccaagggcagccagaagggcacccgccaaaccaaagccgcaaaggaaattggccagtgcgcacccgatccccgacaaggagtcgaggacgaaggcgcgggagagggcgagggagcggactagggaaaagaaccggatgcgatgggtgacgctcgcgtccacaattaacatcgagccggcagccaccggcatggcggcggcgagggcggacgagttcgtcacgaggccgaacaatttgatcagtcgctcctcgtccatgaacacagcaagagctgaattggaggaggggtgctcgtcgtccatgccgagcgaagcgatcatggctggcttcggcaatggaggatacggaagcagcggtaactactaccagtatcagctggagcagcaatgagagctcggtggagtggtgtttcccaactcccagccctactga

SEQ ID NO: 10 - DNA, 1065 bp, triticum turgidum spp. Durum
  tatB1-4B Mutant allele of tatB1-4B
atgtttcctttctatgattcccctagccccatggacttgcccctttaccaacagctgcagatcagccctccctcgccaaaggcgccggatcaccaatccttgctctactaccattcctcccctgcatttgccgccgactccttccaccacagctacctctgtgccggtgccgcgacgcccacgccgcccgcggccgagatcgacaaccagtcgccgccggagctgctgctgatggatcaggctccggcgccaagggcagacggtgttggaaccgcacaaggcctgcacggcggtggaggcctcgacagcgcggcggccgccgcggccaggaaagaccggcacagcaagatatgcaccgccggcgggatgagggaccggcggatgcggctctccctcgacgtcgcccgcaagttcttcgcgctctaggacatgctcggcttcgacaaggccagcaagacggtgcaatggctcctcaatacgtcaaagggcgccatcagggaggtcatgactgacgaggcgtcctccgactgcgaggaggacggctccagcagcctctccgtcgtcgacggcaagcacaagccgccagggacggaggctgaaggtggtgatcacggcgaggggaagaagcaggtgccaagggcagccagaagggcacccgccaaaccaaagccgcaaaggaaattggccagtgcgcacccgatccccgacaaggagtcgaggacgaaggcgcgggagagggcgagggagcggactagggaaaagaaccggatgcgatgggtgacgctcgcgtccacaattaacatcgagccggcagccaccggcatggcggcggcgagggcggacgagttcgtcacgaggccgaacaatttgatcagtcgctcctcgtccatgaacacagcaagagctgaattggaggaggggtgctcgtcgtccatgccgagcgaagcgatcatggctggcttcggcaatggaggatacggaagcagcggtaactactaccagtatcagctggagcagcaatgggagctcggtggagtggtgtttcccaactcccagccctactga

(19) United States
(12) Patent Application Publication
(10) Pub. No.: US 2026/0152756 A1
(43) Pub. Date: Jun. 4, 2026

(54) OPTIMIZATION OF SMALL GRAIN CEREAL FORAGE AND GRAIN PRODUCTION VIA MUTATION OF THE TB1 AND RHT GENES

(71) Applicant: MONTANA STATE UNIVERSITY, Bozeman, MT (US)

(72) Inventor: MICHAEL GIROUX, Bozeman, MT (US)

(21) Appl. No.: 18/271,210

(22) PCT Filed: Jan. 6, 2022
(86) PCT No.: PCT/US22/11455

§ 371 Date: Jul. 6, 2023

Related U.S. Application Data
(60) Provisional application No. 63/134,729, filed on Jan. 7, 2021.

(30) Foreign Application Priority Data
Jan. 7, 2021 (US) 63134729

(51) Int. Cl. C12N 15/82

(52) U.S. Cl. CPC C12N 15/8251

(57) ABSTRACT

Methods of producing a mutated wheat plant having an increased wheat yield content, an increased seed protein content, an increased biomass content, or a combination thereof, are also disclosed comprising: introducing at least one TB1 null allele mutation, at least one Rht semi-dwarfing allele mutation, or a combination thereof to a wheat plant seed to produce a first mutated wheat plant seed, wherein the mutated wheat plant has a modified height as compared to a nonmutated wheat plant.

### GOVERNMENT RIGHTS

This invention was made with government support under contracts 2017-67014-26190 and 2019-67014-29199 awarded by the National Institute of Food and Agriculture, USDA. The government has certain rights in the invention.

### SEQUENCE LISTING

The Sequence Listing found in File Name “SequenceListing01”, created on Jan. 3, 2025 and which is 23.9 KB in size, is incorporated herein by reference.

### FIELD OF THE SUBJECT MATTER

The field of the subject matter is optimization of small grain cereal forage and grain production via mutation of the TB1 and Rht genes. Methods of production and use are also disclosed, along with the development of wheat plants and seeds. The subject matter also relates to identifying Rht and TB1 mutations to optimize small grain cereal crop height, tillering potential, and seed protein content for forage, grain production, and dual-purpose varieties.

### BACKGROUND

Durum wheat, Triticum turgidum ssp. durum, is the tetraploid wheat species used to produce semolina for pasta worldwide, and several specialty bread and dessert products in certain cultures. In the U.S., durum wheat is grown mainly in four states: North Dakota, Montana, California, and Arizona (NASS 2020b).

U.S. durum wheat acres consisted of between 0.46 and 0.98 million hectares planted yearly from 2011 to 2020 (NASS 2020c). Accounting for only 2-4% of the total wheat hectares planted in those years (NASS 2020a), durum wheat acres were worth just a fraction of the overall U.S. wheat crop value. However, to those localities where durum wheat is grown, it is economically important. For example, Montana harvested 550,000 acres of durum wheat in 2019, worth about 104 million dollars (NASS 2019). Breeding program objectives include both regionally important agronomic traits such as race-specific disease resistance and universally desired traits such as grain yield and end-use quality traits like high seed protein content, semolina yield, gluten strength, pasta firmness, and semolina and pasta color (Clark et al. 1998, Royo et al. 2009).

When choosing a variety to grow, farmers consider several factors including yield stability, disease and insect resistance, and seed quality. Not surprisingly, yield is the number one factor since profitability is most affected by yield. Thus, it is important for breeding programs to create and release locally adapted, high yielding durum cultivars. Total grain yield is impacted by genetic and environmental components. Environmental factors including nutrient and water availability, temperature throughout the growing season, competition from weeds, disease and insect pressure impact yield, but genetic factors are what determine the yield potential of a variety under ideal field conditions (Duggan et al. 2000, Destro et al. 2001).

One of the difficulties in selecting for increasing yield is that many yield-impacting traits such as productive tillers, spikelets per head, and seed size are negatively correlated (Fischer et al. 1977, Cantrell and Haro-Arias 1986). As a result, studies designed to increase yield generally focus on one or more of these factors with varying levels of success. Some methods, with a few mentioned below, have a universally beneficial effect. Other studies have shown that what impacts yield under certain environmental conditions has little effect in others (Garcia del Moral et al. 2003, Elhani et al. 2007, Moayedi et al. 2009, Destro et al. 2001).

Increasing the number of tillers per plant, and presumably spikes per plant, can increase yield (Naseer et al. 2016). However, simply increasing total tillers is not sufficient. Late increases in tillering can siphon away limited resources that otherwise would contribute to grain yield, resulting in a plant with few productive tillers and lower grain yield (Elhani et al. 2006 and Motzo et al. 2002). It is necessary, therefore, to increase the number of productive tillers that contribute to yield. An allele identified at a chromosome 6B QTL was associated with increased early tiller number and led to an increase in productive tiller number (PTL) and yield in favorable environments (Naseer et al. 2016). This allele has been identified in several popular Montana grown hard red spring wheat varieties, such as Vida (PI 642366) and Reeder (PI 613586) (Naseer et al. 2016), indicating that manipulation of tiller number is a factor already being utilized in wheat breeding programs. Demonstrating specific genes' effects on tillering is an approach that can be used to modify genetic yield potential.

Tillering determines not only the number of spikes per plant, but also plant canopy size and photosynthetic area. The desired plant phenotype in wheat would optimize tiller number to maximize wheat grain and biomass yield. There is a fine balance between too many tillers and too few. Too few or too many results in loss of grain yield. Wheat tiller development takes place in several stages. These include axillary bud initiation, primary bud outgrowth, tillering cessation, tiller abortion, and finally development of surviving tillers. Tiller buds develop from axillary meristems in the axils of developing leaves from main shoots (Baker and Gallagher, 1983; Longnecker et al., 1993) and tillering normally ends immediately prior to stem elongation (Baker and Gallagher, 1983; Gomez-Macpherson et al., 1998). Each tillering stage is regulated by genetic, metabolic, and environmental factors.

The gene Teosinte Branched 1 (TB1) has been demonstrated to regulate tiller number in wheat in a dosage dependent manner (Dixon et al., 2018), with high expression leading to fewer tillers and increased spikelet number. TB1 was originally discovered in maize, where expression leads to complete loss of axillary buds (Doebley et al., 1997). TB1 orthologs regulate tiller number and/or inflorescence architecture across many cereal crops including maize, barley, rice, and wheat (Doebley et al., 1995; 1997; Takeda et al., 2003; Studer et al., 2011; Ramsay et al., 2011, Dixon et al., 2018).

As a class II member of the transcriptional regulator family known as the TCP family (Cubas et al. 1999), TB1 and its homologs all have conserved function as negative regulators that suppress lateral branching, growth of axillary buds, and internode growth of branches (Aguilar-Martinez et al. 2007, Doebley et al. 1995 and Doebley et al. 1997, and Takeda et al. 2003). However, the effect of TB1 on plant architecture varies across species.

In maize, TB1 prevents outgrowth of buds at lower nodes and promotes development of ears at upper nodes, while mutant TB1 plants develop more tillers and male inflorescences (Cubas et al. 1999). Mutant TB1 alleles in maize were also associated with increased node length and overall plant height (Hubbard et al. 2002) and an increase in root biomass that keeps the ratio of above-ground to below-ground growth proportionately similar across mutant and wild-type genotypes (Gaudin et al. 2014), along with reduced expression of genes in growth and development regulating hormone pathways (Dong et al. 2019).

The gene most closely related to TB1, BRC1, restricts bud outgrowth in Arabidopsis and other dicots (Aguilar-Martinez et al. 2007). Overexpression of OsTB1 suppresses outgrowth by limiting tiller development to the primordial stage in rice (Sakamoto and Matsuoka 2004), while overexpression of PvTB1 in switchgrass inhibited tiller emergence and development, along with inducing changes in stem height and diameter, and biomass yield (Xu et al. 2014). Repressed TB1 expression in sorghum was shown to induce axillary bud growth (Kebrom et al. 2006).

Hexaploid wheat containing TB1 mutant alleles demonstrated to have reduced in vitro binding to the Flowering Time 1 protein were taller than wild type plants (Dixon et al. 2020). Altered bread wheat inflorescence architecture in the form of “paired spikelets” was associated with increased D genome expression of TB1 (Dixon et al. 2018). Lewis et al. (2008) showed that overexpression of TB1 in wheat results in fewer tillers, reduced axillary bud outgrowth, shorter plants, and increased inflorescence axillary outgrowth. Additionally, overexpression of the taemiR156 regulator resulted in reduced expression of TaTB1 and enhanced hexaploid wheat tillering (Liu et al. 2018).

TB1 has been shown to be an important gene contributing to crop yield. Investigation of this gene has focused on how this gene regulates cereal inflorescence architecture. In wheat and other grasses, inflorescence architecture is made up of flowers arranged on branches known as spikelets. The number and arrangement of flowers and spikelets within the inflorescence can contribute to grain yield as this trait determines the number of sites available for seed development. Wheat has a central rachis with multiple spikelets alternating on opposite sides of the rachis. Each spikelet produces multiple florets, with both spikelet and floret number contributing to grain yield. Until recently, there has been little research regarding how this trait is genetically regulated.

Aside from the wild type (WT) TB1 alleles (TB-A1a, TB-B1a, and TB-D1a), only two other variant alleles have been identified in modern wheat germplasm (Dixon et al., 2018). For TB-D1, a single nonsynonymous point mutation has been identified at the beginning of the TCP domain in TB-D1. The second variant is in TB-B1 and contains five sequence polymorphisms. These alleles lend to the diversity of TB1 in modern wheat varieties, although while these alleles may have altered TB1 function neither has been demonstrated to be associated with increased tillering or plant biomass. It is likely, due to the nature of each of those mutations, that any influence they may have on plant yield is limited. Missense mutations do not as a rule lead to large changes in protein function. In our research we have identified stop codon (nonsense) mutations in TB1 to knock down function of TB1 and increase tiller number and plant biomass. Note that the focus of published research to date has been on the effect that TB1 has on inflorescence architecture in wheat, rather than the role it plays in tillering potential. No published data seems to exist on seed protein content and seed size.

Another mutation that has interest with respect to the work disclosed herein, is the Rht gene or Reduced Height gene mutation. The ability to increase wheat yield while not reducing protein content and product quality is paramount to increasing economic returns for wheat growers. Historically, one of the greatest successes in this endeavor was the introduction of dwarfing genes into cereals as part of the ‘green revolution.’ Modern reduced height (semi-dwarf) varieties have approximately 10-14% higher yields compared to standard (full height) wheat varieties. This results from both decreased loss of seed yield from lodging, and from a redirecting of nutrients from stem development to the spike, resulting in increased seed number per head (Youseffian et al., 1992) and increased productive tiller number (Allan, 1986).

Incorporation of semi-dwarfing alleles of the Reduced Height (Rht) gene into hexaploid wheat (Triticum aestivum) varieties led to increased yield during the middle part of the 20^{th} century (Hedden 2003, Pearce et al. 2011). Mutant alleles Rht-B1b and Rht-D1b have nearly identical impacts on hexaploid wheat height and grain yield. Each confers a height reduction of ˜20% and yield increase of ˜10% (Hoogendoorn et al. 1990, Flintham et al. 1997). Most modern hexaploid wheat varieties are semi-dwarf and contain either Rht-B1b or Rht-D1b (Worland et. al. 1998, Knopf et al. 2008). The Rht semi-dwarfing alleles increase seeds per plant and productive tiller number, which result in increased yield (Flintham et al. 1997, Hoogendoorn et al. 1990, Li et al. 2006, Pearce et al. 2011, Peng et al. 1999). Rht-B1b and Rht-D1b also confer a decrease in seed size and protein, along with reducing coleoptile and stem internode length (Fick and Qualset 1979, Schillinger et al. 1998, Jobson et al. in press 2020, Amram et al. 2015, Liatukas and Ruzgas 2011).

In the semi-dominant, altered function Rht-B1b and Rht-D1b alleles, a premature stop codon in the N-terminal DELLA domain prevents GID1 (GIBBERELLIN INSENSITIVE DWARF1, a receptor protein) from binding (Peng et al. 1999, Pearce et al. 2011). It is proposed that Rht translation reinitiates after the stop codon, which produces N-terminally truncated DELLA proteins (Mo et al. 2018). These shorter DELLA proteins cannot detect GID1-mediated gibberellic acid (GA) signals, and plants are rendered partially GA insensitive (Gale & Marshall 1973, Peng et al. 1999, Youssefian et al. 1992). This insensitivity confers the semi-dwarf phenotype seen in Rht-B1b and Rht-D1b hexaploid wheat plants.

Genes belonging to the DELLA domain containing gene family play key roles in GA signaling pathways and are present in many plant species. Characterized orthologs of wheats' Rht-1 genes include GAI and RGA (Arabidopsis), Slender1 (SLN1 in barley; SLR1 in rice), and Dwarf 8 and Dwarf 9 (maize) (Wilhem et al. 2013, Ikeda et al. 2001). Each of these acts in a similar manner to Rht-1, with mutant alleles causing reduced GA responsiveness and plant growth (Peng et al. 1997, Chandler et al. 2008, Ikeda et al. 2001, Winkler and Freeling 1994, Chandler et al. 2002). Other Poeacae orthologs include Bradi1g11090.1 (B. distachyon), Si039400m (foxtail millet), Sb01g010660 (sorghum), GenBank accession #JN793959 (E. tef), Dwarf8 and Dwarf9 (maize), and GenBank accession #DQ062091.1 (sugarcane) (Wilhelm et al. 2013). Of the listed orthologs, 80-95% of the protein residues are shared with Rht-1 in Chinese Spring wheat (Wilhelm et al. 2013).

Much research has focused on the effects of Rht-B1b and Rht-D1b in hexaploid wheat, but less is known about the impact of Rht alleles in durum wheat. Decreasing plant height has been shown to increase yield in durum wheat as well, with the commonly used Rht-B1b semi dwarfing gene increasing yield by ˜8.7% in durum wheat (Mathews et al. 2006). However, this moderate yield increase comes at the cost of a severe decrease in height of over 45% (Mathews et. al. 2006). Plants that are too short present harvesting challenges. The combine header must be lowered enough to capture the plant heads, and if the moving metal parts strike a rock there is both fire danger and possible equipment damage. Additionally, short coleoptiles are associated with poor emergence when seeds are planted deeper to reach the soil moisture, as is common in dry climates (Schillinger et al. 1998). In Montana, only one of the commonly grown durum varieties carries the Rht-B1b semi-dwarfing allele. This provides an opportunity for a breeding program to create varieties with a more moderate height decrease but increased yield potential over the currently grown full-height varieties.

Mutations in the Reduced Height (Rht) genes impact wheat plant height (Allan, 1970). There are three functional copies of Rht in wheat, one each on the group 4 A, B, and D chromosomes (Gale et al., 1975; Gale and Marshall, 1976; McVittie et al., 1978; Sourdille et al., 1998). Wheat varieties carrying Rht mutations have increased yield relative to standard height varieties (Butler et al. 2005, Blake et al. 2009, Lanning et al. 2012). Select dominant acting mutations in the B and D genome copies result in reduced height (no Rht-A1a semi-dwarfing mutations have been reported) (Prior Art FIG. 2). The Japanese wheat variety Norin 10 (Reitz and Salmon, 1968), used in the green revolution, contains the Rht-B1b and the Rht-D1b mutations (Borner, et al., 1996), and most semi-dwarf wheat varieties contain one of these two alleles. There is no measurable functional difference between Rht-B1b and Rht-D1b. Both produce a 15-20% reduction in plant height and a ˜10-14% increase in wheat grain yield (Gent et al., 1997).

Essentially all hexaploid semi-dwarf wheat varieties carry either Rht-B1b or Rht-D1b (Evans et al., 1998). Presence of Rht-B1b or Rht-D1b reduces plant height by ˜20% (Gale and Youseffian, 1985; Flintham et al., 1997, Jobson et al., in press 2019). This trait is observable shortly after germination, with reduced height lines having shorter coleoptiles (Trethowan et al., 2001). Although other mutations in Rht have been identified in wheat, they do not result in useful plant phenotypes. Rht mutations such as Rht-Blc and Rht-D1c or the combination of Rht-B1b and Rht-D1b result in wheat that is not as high yielding as Rht-B1b or Rht-D1b, or wheat that is too short to be readily harvested (Hoogendoorn et al., 1990; Flintham et al., 1997) (Prior Art FIG. 2). Other sources of height reduction and yield increase in wheat, such as Rht8, are inferior in yield relative to Rht-B1b or Rht-D1b (Lanning et al., 2012).

Selecting for mutations in genes that both reduce height and increase productive tillers is known to increase yield, as seen with the commonly used Rht-B1b semi-dwarfing gene which increases yield by ˜8.7% in durum wheat (Mathews et al. 2006). With a reduction in height, the plant can redirect the energy that would have been expended on stem growth to seed production instead (Youseffian et al. 1992, Flintham et al. 1997). Although Rht-B1b is well studied and is effective at reducing height, increasing productive tillers, and increasing yield in hexaploid wheat (Flintham et al. 1997, Hoogendoorn et al. 1990, Li et al. 2006, Pearce et al. 2011, Peng et al. 1999, Fick and Qualset 1979, Jobson et al. 2018, Jobson et al. 2019), there still exists opportunities for other variation in Rht that may have more practical applications in tetraploid wheat. The effect of Rht-B1b in durum is greater than in hexaploid wheat (Mathews et al. 2006), so a less dramatic height reduction allele would be useful for breeding programs.

By exploring the effect of Rht-A mutations in durum wheat both with and without the Rht-B1b allele, we hope to identify an allele that would confer height intermediate between standard and semi-dwarf plant height. Along with Rht-related yield increases from reduced and/or modified height and increased productive tillers, other genes could also provide a yield increase through similar mechanisms. Genes that are known to affect tillering, such as Teosinte Branched 1 (TB1), could be manipulated to increase the number of tillers a plant produces (Dixon et al. 2018). An increase in total tiller number, when associated with an increase in productive tiller number, can increase plant yield (Destro et al. 2001, Elhani et al. 2007).

To this end, it would be desirable to optimize small grain cereal forage and grain production by: a) identifying TB1 mutations and testing their impact on wheat tillering potential, plant biomass, seed size, protein content, and grain yield; b) demonstrating that alleles with altered or mutated TB1 structure are be associated with increased tillering or plant biomass; c) identifying RHT mutations and testing their impact on plant biomass, seed size, protein content, and grain yield; and d) demonstrating that alleles with altered or mutated RHT structure are associated with height variation, increased yield, and either the same or an increased seed protein content.

### SUMMARY OF THE SUBJECT MATTER

Methods of producing a mutated wheat plant having an increased biomass content, an increased seed protein content, or a combination thereof are disclosed comprising: introducing at least one TB1 null allele mutation to a wheat plant seed to produce a first TB1 mutated wheat plant seed.

Methods of producing a mutated wheat plant having an increased wheat yield content, are disclosed comprising: introducing at least one Rht semi-dwarfing allele mutation to a wheat plant seed to produce a first Rht mutated wheat plant seed, wherein the mutated wheat plant has a modified height as compared to a nonmutated wheat plant.

Methods of producing a mutated wheat plant having an increased wheat yield content, an increased seed protein content, an increased biomass content, or a combination thereof, are also disclosed comprising: introducing at least one TB1 null allele mutation, at least one Rht semi-dwarfing allele mutation, or a combination thereof to a wheat plant seed to produce a first mutated wheat plant seed, wherein the mutated wheat plant has a modified height as compared to a nonmutated wheat plant.

Methods of producing a mutated wheat plant having an increased wheat yield content, an increased seed protein content, an increased biomass content, or a combination thereof, are disclosed comprising: introducing at least one TB1 null allele mutation, at least one Rht semi-dwarfing allele mutation, or a combination thereof to produce a mutated wheat plant having an increased wheat yield content; introducing at least one second TB1 null allele mutation, at least one second Rht semi-dwarfing allele mutation, or a combination thereof to a wheat plant seed to produce a second mutated wheat plant seed; crossing the second mutated wheat plant seed with the first mutated wheat plant seed, and producing a third mutated wheat plant seed.

Mutated wheat plant seeds, mutated wheat plants, or a combination thereof produced from contemplated methods are also disclosed.

### BRIEF DESCRIPTION OF THE FIGURES

Prior Art FIG. 1 shows the impact of the highly branched (hb) allele upon wheat plants. Inflorescence architecture (A), frequency of paired spikelets (B), tiller phenotype (C), and tiller number (D) in near isogenic lines (NILs) with either wild type or ‘highly branched’ (hb) inflorescences with multiple paired spikelets (Dixon et al., 2018). (The hb allele has increased dosage and expression of TB-D1 in developing inflorescences relative to the wild type.)

Prior Art FIG. 2 shows the effects of different dominant acting Rht alleles on plant height in wheat (cv. April Bearded). Standard height (wild-type) wheat varieties contain the Rht-B1a and Rht-D1a alleles (a), located on the B and D genomes, respectively. Modern semi-dwarf varieties contain either the Rht-Blb (b) or Rht-D1b (c) allele. The combination of Rht-B1b and Rht-D1b (d) results in a dwarfing phenotype too severe to harvest. Rht-B1c is also a very severe mutation (e). Combinations of Rht alleles result in an additive effect based on mutation severity (d, f). Figure from Hedden, (2010).

FIG. 3 shows the average plant height of progeny lines containing Rht-A1/Rht-B1b-E529K, Rht-A1-S50T/Rht-B1b, or Rht-A1-S358F/Rht-B1b with each compared to control progeny lines carrying Rht-A1a, Rht-B1b (standard semidwarf). Rht-B1b-E529K increases plant height while S50T and L358F do not alter plant height in the presence of Rht-B1b.

FIG. 4 shows the average seed weight of progeny lines between durum varieties containing Rht-A1/Rht-B1b-E529K, Rht-A1-S50T/Rht-B1b, or Rht-A1-S358F/Rht-B1b with each compared to control progeny lines carrying Rht-A1a, Rht-B1b (standard semidwarf). Rht-A1-S50T increases plant yield while L358F did not alter plant yield in the presence of Rht-B1b.

FIG. 5 shows the average plant height of progeny lines containing Rht-A1/Rht-B1b-E529K, Rht-A1-S50T/Rht-B1a, or Rht-A1-S358F/Rht-B1a. All three combinations decreased plant height relative to Rht-A1a/Rht-B1a and all created specific plant heights.

FIG. 6 shows the average plant yield of progeny lines containing Rht-A1/Rht-B1b-E529K, Rht-A1-S50T/Rht-B1a, or Rht-A1-S358F/Rht-B1a. Only Rht-A1-S50T and Rht-A1-L358F had distinct trends of increased yield relative to Rht-A1, Rht-B1a.

FIG. 7 shows Yeast-2-hybrid assay of durum EMS-derived Rht mutations to characterize the interaction between DELLA and GID1 in the presence or absence of 100 μM GA.

FIG. 8 shows Yeast-2-hybrid assay of EMS-derived Rht mutations found in hexaploid wheat indicating the interaction between DELLA and GID1 in the presence or absence of 100 μM GA.

### SEQUENCES

Below is a brief description of certain sequences described herein.

(SEQ ID NO 001)
>Rht-A1a

Type: DNA
Organism: Triticum turgidum spp. Durum
Length: 1863 base pairs
Definition: The Rht-Ala Wild Type Allele of Rht-A1

(SEQ ID NO 002)
>Rht-A1-MDIV169-L358F

Type: DNA
Organism: Triticum turgidum spp. Durum
Length: 1863 base pairs
Definition: Mutant Allele of Rht-Ala
Location: Mutation at the 1072 base pair
Other Information: Base change from C to T resulting in amino acid change L358F

(SEQ ID NO 003)
>Rht-A1-MDIV4-S50F

Type: DNA
Organism: Triticum turgidum spp. Durum
Length: 1863 base pairs
Definition: Mutant Allele of Rht-Ala
Location: Mutation at the 149 base pair
Other Information: Base change from C to T resulting in amino acid change S50F

(SEQ ID NO 004)
>Rht-B1a

Type: DNA
Organism: Triticum turgidum spp. Durum
Length: 1866 base pairs
Definition: The Rht-B1a Wild Type Allele of Rht-B1

(SEQ ID NO 005)
>Rht-B1b-Kronos-E529K

Type: DNA
Organism: Triticum turgidum spp. Durum
Length: 1866 base pairs
Definition: Mutant Allele of Rht-B1b
Location: Mutation at the 1585 base pair
Other Information: Base change from C to T resulting in amino acid change E529K

(SEQ ID NO 006)
>TaTB1-4A-Wildtype

Type: DNA
Organism: Triticum turgidum spp. Durum
Length: 1059 base pairs
Definition: The TaTB1-4A Wild Type Allele of TaTB1-4A

(SEQ ID NO 007)
>TaTB1-4A-Kronos 944-STOP

Type: DNA
Organism: Triticum turgidum spp. Durum
Length: 1059 base pairs
Definition: Mutant Allele of TaTB1-4A
Location: Mutation at the 1016 base pair

(SEQ ID NO 008)
>TaTB1-4B-Wildtype

Type: DNA
Organism: Triticum turgidum spp. Durum
Length: 1065 base pairs
Definition: The TaTB1-4B Wild Type Allele of TaTB1-4B

(SEQ ID NO 009)
>TaTB1-4B-Kronos562-STOP

Type: DNA
Organism: Triticum turgidum spp. Durum
Length: 1065 base pairs
Definition: Mutant Allele of TaTB1-4B
Location: Mutation at the 1023 base pair

(SEQ ID NO 010)
>TaTB1-4B-Kronos2226-STOP

Type: DNA
Organism: Triticum turgidum spp. Durum
Length: 1065 base pairs
Definition: Mutant Allele of TaTB1-4B
Location: Mutation at the 424 base pair

### DETAILED DESCRIPTION

As part of this work, contemplated and disclosed embodiments have shown the ability to optimize small grain cereal forage and grain production by: a) identifying TB1 mutations and testing their impact on wheat tillering potential, plant biomass, seed size, protein content, and grain yield; b) demonstrating that alleles with altered or mutated TB1 structure are associated with increased tillering or plant biomass; c) identifying RHT mutations and testing their impact on plant biomass, seed size, protein content, and grain yield; and d) demonstrating that alleles with altered or mutated RHT structure are associated with height variation—either increased or reduced, increased yield, and either the same or an increased seed protein content. Each of these outcomes will be described in detail herein, along with showing examples and data, so that one of ordinary skill in the art can understand the importance of each of these outcomes and why this work is important, novel, and inventive in light of conventional methods and approaches.

Increasing the yield of wheat requires identifying new genetic combinations of alleles by crossing or by creating variation in yield limiting genes. Wheat yield is impacted by tiller number and seeds per tiller, both of which are impacted by the Reduced height (Rht) and Teosinte branched1 (TB1) genes. As part of this work, wheat plants, including durum plants, varying for Rht and TB1 alleles were created by EMS (ethyl methanesulfonate) mutagenesis and were studied to determine the impact of each allele upon agronomic and seed traits. Both projects aimed to increase durum yield, one through an increase in tiller number; the other through the development of a plant with height between current full-height and semidwarf varieties that can allocate more resources to seed production.

The impact of TB1 null alleles were studied alone and together in greenhouse and field trials. TB1 single and double null mutants produced more biomass and tillers per plant, and expression of genes in meristems varied. A screen of wheat varieties grown in Montana identified that several spring and durum wheat varieties contain different TB1 alleles, but none contained TB1 null alleles.

Rht experiments included field trials, coleoptile length and gibberellic acid responsiveness assays, and an in-vitro test to determine the impact of each Rht mutation upon binding to Gibberellin Interacting Domain 1 (GID1) that directly influences plant height. It was found that the previously described Rht-B1b-E529K allele reduced both plant height and coleoptile length while two newly characterized Rht mutations had lesser impacts with trends towards intermediate-height plants. The results of this research demonstrate that Rht alleles that alter RHT binding to GID1 and TB1 null alleles prove useful in increasing durum tillering and optimizing plant height for different growing conditions.

There are several benefits to utilizing a TB1 gene mutation, a RHT gene mutation or a combination thereof, including: increase grain and/or forage yield, while maintaining or even increasing seed protein content; non-genetically modified (Non-GMO) solutions to optimize variety performance; Rht and TB1 discoveries can be stacked to fine tune varieties; ability to produce intermediate height durum varieties that are optimized for prime growing regions in North America, including Montana, North Dakota, and Canada; potential to improve forage yields by at least 18% without sacrificing seed yield or quality in dual purpose varieties.

With respect to the TB1 gene mutations, investigation of the ‘paired spikelets’ (or ‘highly branched’ (hb)) trait has been particularly informative. Notably, the increased spikelet trait is negatively correlated with tiller number (Prior Art FIG. 1). Based upon this and upon our preliminary results we hypothesized that combining loss of function TB1 mutations through crossing would increase tillering and reduce paired spikelet formation. Reducing paired spikelet formation could also increase protein content and seed size. In fact, our results demonstrate that this approach is feasible, and that TB1 mutations increase tiller number and plant biomass without negatively impacting seed protein content. Increasing productive tiller number will prove valuable to increasing plant biomass and forage yield. At the same time reducing the number of paired spikelets will reduce seed size variation, which is an added benefit in wheat.

Specifically, and as contemplated herein, methods of producing a wheat plant having an increased biomass content are disclosed and described herein comprising: introducing at least one TB1 null allele mutation to a wheat plant seed to produce a first TB1 mutated wheat plant seed. Contemplated methods further comprise introducing a second TB1 null allele mutation to a wheat plant seed to produce a second TB1 mutated wheat plant seed, crossing the second TB1 mutated wheat plant seed with the first TB1 mutated wheat plant seed, and producing a third TB1 mutated wheat plant seed. Mutated wheat plants produced from these methods are also contemplated. In addition, mutated wheat plant seeds produced from these methods are contemplated.

In contemplated embodiments, the biomass content of a mutated wheat plant is increased by at least 5% as compared to or with the biomass content of a non-mutated wheat plant. In additional contemplated embodiments, the biomass content of a mutated wheat plant is increased by at least 10% as compared to or with the biomass content of a non-mutated wheat plant. In yet other contemplated embodiments, the biomass content of a mutated wheat plant is increased by at least 15% as compared to or with the biomass content of a non-mutated wheat plant. Biomass content is measured and quantified by the total dry weight of the harvested plant tissue.

It should be understood as used herein and throughout this work that the phrase “non-mutated wheat plant” means a biosimilar wheat plant as the mutated wheat plant other than the mutation that has been introduced. In other words, the comparison between a mutated wheat plant and a non-mutated wheat plant is a comparison between two biosimilar wheat plants and not of two different varieties.

Specifically, and as contemplated herein, methods of producing a wheat plant having increased seed protein content are disclosed and described herein comprising: introducing at least one TB1 null allele mutation to a wheat plant seed to produce a first TB1 mutated wheat plant seed. Contemplated methods further comprise introducing a second TB1 null allele mutation to a wheat plant seed to produce a second TB1 mutated wheat plant seed, crossing the second TB1 mutated wheat plant seed with the first TB1 mutated wheat plant seed, and producing a third TB1 mutated wheat plant seed. Mutated wheat plants produced from these methods are also contemplated. In addition, mutated wheat plant seeds produced from these methods are contemplated. In some embodiments, there may be multiple mutations in one TB1 gene. In other embodiments, there may be mutations in different copies of the TB1 gene with one on each genome. In yet other contemplated embodiments, mutations are combined in independent copies of the TB1 gene, where there is one copy on each genome.

In contemplated embodiments, the seed protein content of a mutated wheat plant is increased by at least 1% as compared to or with the seed protein content of a non-mutated wheat plant. In additional contemplated embodiments, the seed protein content of a mutated wheat plant is increased by at least 3% as compared to or with the seed protein content of a non-mutated wheat plant. In yet other contemplated embodiments, the seed protein content of a mutated wheat plant is increased by at least 5% as compared to or with the seed protein content of a non-mutated wheat plant. Seed protein content is measured and quantified by a weight percent of the seed.

Specifically, and as contemplated herein, methods of producing a wheat plant having an increased biomass content and an increased seed protein content are disclosed and described herein comprise: introducing at least one TB1 null allele mutation to a wheat plant seed to produce a first TB1 mutated wheat plant seed. Contemplated methods further comprise introducing a second TB1 null allele mutation to a wheat plant seed to produce a second TB1 mutated wheat plant seed, crossing the second TB1 mutated wheat plant seed with the first TB1 mutated wheat plant seed, and producing a third TB1 mutated wheat plant seed. Mutated wheat plants produced from these methods are also contemplated. In addition, mutated wheat plant seeds produced from these methods are contemplated. For the purposes of this application, the term “biomass” and the phrase “biomass content” mean the same thing and can be used interchangeably with one other to mean the biomass or amount of biomass.

In contemplated embodiments, the biomass content of a mutated wheat plant is increased by at least 5% as compared to or with the biomass content of a non-mutated wheat plant and the seed protein content of the mutated wheat plant is increased at least 1% as compared to the seed protein content of a nonmutated wheat plant. In additional contemplated embodiments, the biomass content of a mutated wheat plant is increased by at least 10% as compared to or with the biomass content of a non-mutated wheat plant and the seed protein content of the mutated wheat plant is increased at least 3% as compared to the seed protein content of a nonmutated wheat plant. In yet other contemplated embodiments, the biomass content of a mutated wheat plant is increased by at least 15% as compared to or with the biomass content of a non-mutated wheat plant and the seed protein content of the mutated wheat plant is increased at least 5% as compared to the seed protein content of a nonmutated wheat plant.

As contemplated herein, TB1 null allele mutations are found in genomes in the group consisting of SEQ ID NO. 007 and 009-010, which are described herein and incorporated herein by reference.

In addition, and as contemplated herein, methods of producing a mutated wheat plant having an increased wheat yield content, comprise: introducing at least one Rht semi-dwarfing allele mutation to a wheat plant seed to produce a first Rht mutated wheat plant seed, wherein the mutated wheat plant has a modified (either increased or decreased/reduced) height as compared to a nonmutated wheat plant. In some embodiments, methods of producing a mutated wheat plant having an increased wheat yield content, comprise: introducing a second Rht semi-dwarfing allele mutation to a wheat plant seed to produce a second Rht mutated wheat plant seed, crossing the second Rht mutated wheat plant seed with the first Rht mutated wheat plant seed, and producing a third Rht mutated wheat plant seed. Mutated wheat plants produced from these methods are also contemplated. In addition, mutated wheat plant seeds produced from these methods are contemplated.

In contemplated methods, the wheat yield content of the mutated wheat plant is increased at least 5% as compared to the yield content of a non-mutated wheat plant. In other contemplated methods, the yield content of the mutated wheat plant is increased at least 10% as compared to the yield content of a non-mutated wheat plant. In yet other contemplated methods, the yield content of the mutated wheat plant is increased at least 20% as compared to the yield content of a non-mutated wheat plant. In contemplated embodiments, the at least one Rht semi-dwarfing allele mutation is found in a genome in the group consisting of SEQ ID NO. 002, 003, and 005.

In contemplated methods, the modification in height of the mutated wheat plant can be either increased or reduced by at least 5%, as compared to the average height of a non-mutated wheat plant. In other contemplated methods, the height of the mutated wheat plant can be either increased or reduced by at least 10%, as compared to the height of a non-mutated wheat plant. In yet other contemplated methods, the height of the mutated wheat plant can be either increased or reduced by at least 20%, as compared to the height of a non-mutated wheat plant. In contemplated embodiments, the at least one Rht semi-dwarfing allele mutation is found in a genome in the group consisting of SEQ ID NO. 002, 003, and 005. It should be noted that in some instances, as disclosed herein, the height is increased. For example, the Rht-B1b-E529K allele is increased in height relative to Rht-B1b.

As also contemplated herein, methods of producing a mutated wheat plant having an increased wheat yield content, an increased seed protein content, an increased biomass content, or a combination thereof, comprise: introducing at least one TB1 null allele mutation, at least one Rht semi-dwarfing allele mutation, or a combination thereof to a wheat plant seed to produce a first mutated wheat plant seed, wherein the mutated wheat plant has a modified height as compared to a nonmutated wheat plant. In some embodiments, methods of producing a mutated wheat plant having an increased wheat yield content, an increased seed protein content, an increased biomass, or a combination thereof, comprise: introducing at least one TB1 null allele mutation, at least one Rht semi-dwarfing allele mutation, or a combination thereof to produce a mutated wheat plant having an increased wheat yield content, comprise: introducing at least one second TB1 null allele mutation, at least one second Rht semi-dwarfing allele mutation, or a combination thereof to a wheat plant seed to produce a second mutated wheat plant seed, crossing the second mutated wheat plant seed with the first mutated wheat plant seed, and producing a third mutated wheat plant seed. Mutated wheat plants produced from these methods are also contemplated. In addition, mutated wheat plant seeds produced from these methods are contemplated.

Methods for producing or constructing a mutated wheat plant wherein the mutated wheat plant has increased biomass content are contemplated with the contemplated methods comprising: (i) introducing at least one TB1 null allele mutation into a wheat plant to provide a mutated wheat plant; (ii) selecting the mutated wheat plant that comprises at least one TB1 null allele mutation, thereby constructing the mutated wheat plant wherein the mutated wheat plant exhibits increased biomass content as compared to a non-mutated wheat plant. Mutated wheat plants produced from these methods are also contemplated. In addition, mutated wheat plant seeds produced from these methods are contemplated.

Methods for producing or constructing a mutated wheat plant wherein the mutated wheat plant has increased seed protein content are contemplated with the contemplated methods comprising: (i) introducing at least one TB1 null allele mutation into a wheat plant to provide a mutated wheat plant; (ii) selecting the mutated wheat plant that comprises at least one TB1 null allele mutation, thereby constructing the mutated wheat plant wherein the mutated wheat plant exhibits increased seed protein content as compared to a non-mutated wheat plant. Mutated wheat plants produced from these methods are also contemplated. In addition, mutated wheat plant seeds produced from these methods are contemplated.

Methods for producing or constructing a mutated wheat plant wherein the mutated wheat plant has increased biomass content and increased seed protein content are contemplated with the contemplated methods comprising: (i) introducing at least one TB1 null allele mutation into a wheat plant to provide a mutated wheat plant; (ii) selecting the mutated wheat plant that comprises at least one TB1 null allele mutation, thereby constructing the mutated wheat plant wherein the mutated wheat plant exhibits increased biomass content and increased seed protein content as compared to a non-mutated wheat plant. Mutated wheat plants produced from these methods are also contemplated. In addition, mutated wheat plant seeds produced from these methods are contemplated.

Methods for producing a wheat plant having increased biomass, increased seed protein content, or a combination thereof are disclosed herein, contemplated methods comprising constructing a wheat plant that includes at least one TB1 null allele mutation, to provide a wheat plant having at least one TB1 null allele mutation, thereby producing wheat plants having increased biomass, increased seed protein content, or a combination thereof.

Methods of altering at least one plant architecture characteristic in a wheat plant, comprising: (a) introducing into a regenerable wheat plant cell at least one TB1 null allele mutation; (b) regenerating a transgenic plant from the regenerable plant cell after step (a), wherein the transgenic plant comprises in its genome the at least one TB1 null allele mutation; and (c) obtaining a progeny plant derived from the transgenic plant of step (b), wherein said progeny plant comprises in its genome the at least one TB1 null allele mutation and exhibits an alteration in at least one plant architecture characteristic when compared to a control plant not comprising the at least one TB1 null allele mutation. Mutated wheat plants produced from these methods are also contemplated. In addition, mutated wheat plant seeds produced from these methods are contemplated.

Methods for producing or constructing a mutated wheat plant wherein the mutated wheat plant has an increased wheat yield content are contemplated with the contemplated methods comprising: (i) introducing at least one Rht semi-dwarfing allele mutation into a wheat plant to provide a mutated wheat plant; (ii) selecting the mutated wheat plant that comprises at least one Rht semi-dwarfing allele mutation, thereby constructing the mutated wheat plant wherein the mutated wheat plant exhibits an increased wheat yield content as compared to a non-mutated wheat plant. Mutated wheat plants produced from these methods are also contemplated. In addition, mutated wheat plant seeds produced from these methods are contemplated.

In contemplated methods, the wheat yield content of the mutated wheat plant is increased at least 5% as compared to the yield content of a non-mutated wheat plant. In other contemplated methods, the yield content of the mutated wheat plant is increased at least 10% as compared to the yield content of a non-mutated wheat plant. In yet other contemplated methods, the yield content of the mutated wheat plant is increased at least 20% as compared to the yield content of a non-mutated wheat plant. In contemplated embodiments, the at least one Rht semi-dwarfing allele mutation is found in a genome in the group consisting of SEQ ID NO. 002, 003, and 005.

Methods for producing or constructing a mutated wheat plant wherein the mutated wheat plant has a modified height are contemplated with the contemplated methods comprising: (i) introducing at least one Rht semi-dwarfing allele mutation into a wheat plant to provide a mutated wheat plant; (ii) selecting the mutated wheat plant that comprises at least one Rht semi-dwarfing allele mutation, thereby constructing the mutated wheat plant wherein the mutated wheat plant exhibits a modified height as compared to a non-mutated wheat plant. Mutated wheat plants produced from these methods are also contemplated. In addition, mutated wheat plant seeds produced from these methods are contemplated. In some embodiments, the height of the mutated wheat plant is increased, as compared to a non-mutated wheat plant. In other embodiments, the height of the mutated wheat plant is decreased or reduced, as compared to a non-mutated wheat plant.

In these contemplated methods, the modification in height of the mutated wheat plant can be either increased or reduced by at least 5%, as compared to the average height of a non-mutated wheat plant. In other contemplated methods, the height of the mutated wheat plant can be either increased or reduced by at least 10%, as compared to the height of a non-mutated wheat plant. In yet other contemplated methods, the height of the mutated wheat plant can be either increased or reduced by at least 20%, as compared to the height of a non-mutated wheat plant. In contemplated embodiments, the at least one Rht semi-dwarfing allele mutation is found in a genome in the group consisting of SEQ ID NO. 002, 003, and 005. It should be noted that in some instances, as disclosed herein, the height is increased. For example, the Rht-B1b-E529K allele is increased in height relative to Rht-B1b.

Methods for producing or constructing a mutated wheat plant wherein the mutated wheat plant has an wheat yield content and a modified (either increased or reduced/decreased) height are contemplated with the contemplated methods comprising: (i) introducing at least one Rht semi-dwarfing allele mutation into a wheat plant to provide a mutated wheat plant; (ii) selecting the mutated wheat plant that comprises at least one Rht semi-dwarfing allele mutation, thereby constructing the mutated wheat plant wherein the mutated wheat plant exhibits an increased wheat yield content and a decreased height as compared to a non-mutated wheat plant. Mutated wheat plants produced from these methods are also contemplated. In addition, mutated wheat plant seeds produced from these methods are contemplated.

Methods for producing a wheat plant having an increased wheat yield content, a decreased height, or a combination thereof are disclosed herein, contemplated methods comprising constructing a wheat plant that includes at least one Rht semi-dwarfing allele mutation, to provide a wheat plant having at least one Rht semi-dwarfing allele mutation, thereby producing wheat plants having an increased wheat yield content, a decreased height, or a combination thereof.

Methods of altering at least one plant architecture characteristic in a wheat plant, comprising: (a) introducing into a regenerable wheat plant cell at least one Rht semi-dwarfing allele mutation; (b) regenerating a transgenic plant from the regenerable plant cell after step (a), wherein the transgenic plant comprises in its genome the at least one Rht semi-dwarfing allele mutation; and (c) obtaining a progeny plant derived from the transgenic plant of step (b), wherein said progeny plant comprises in its genome the at least one Rht semi-dwarfing allele mutation and exhibits an alteration in at least one plant architecture characteristic when compared to a control plant not comprising the at least one Rht semi-dwarfing allele mutation. Mutated wheat plants produced from these methods are also contemplated. In addition, mutated wheat plant seeds produced from these methods are contemplated.

Contemplated wheat plants may comprise any wheat relative species, including winter wheat and spring wheat, because of the similarity between durum wheat and hexaploid wheat. In additional embodiments, a contemplated wheat species may be triticale. One of ordinary skill in the art would recognize that the contemplated embodiments disclosed herein can be utilized on other wheat relative species without undue experimentation.

It is important to understand that the contemplated embodiments and work disclosed herein is primarily driven by the “dosage” and number of mutations with respect to the impact that those mutations have on the overall characteristics of the seed or plant. For example, the ratio of wild type alleles to mutant alleles impacts the effect of a mutant allele, since as the proportion of wild type allele decreases the impact of mutation increases. As already discussed herein, Rht and TB1 are linked, so that if the mutations needed are on the same genome, then they should be created in a genotype already carrying the desired wild type or mutant genome.

As used herein, the terms “dosage”, “dose”, and/or “dosing” refers to the number of wild type to semi-dwarfing versions of the Rht and TB1 genes. What is referred to as spring and winter wheat is common hexaploid wheat (Triticum aestivum L.) in which there are three separate genomes (termed A, B, and D) where there exists a functional version of Rht and TB1 on each genome. The commonly used semi-dwarfing alleles of Rht (Rht-B1b and Rht-D1b) reduce hexaploid wheat by 20% when only one is present and there remains two wild type forms of Rht. However, when combined in spring or winter wheat, they reduce plant height more than 50%. Similarly, in durum wheat (Triticum durum L.) which is tetraploid, there are only two separate genomes (A and B) and when Rht-B1b is present, there remains only one functional copy of Rht and height is reduced by at least 45%. Thus, the ratio of semi-dwarfing alleles to remaining wild type versions determines the degree of height reduction.

More specifically, for Rht and hexaploid wheat (wheat with 3 complete sets of homologous chromosomes), one “dose” of a natural semi-dwarfing allele or alleles (either Rht-B1b or RhtD1b) may be appropriate in terms of decreasing height and increasing productive tillers, but it might reduce the seed size and protein content too much. An intermediate height allele might be considered better for seed size and protein content.

For Rht and tetraploid wheat (wheat with 2 complete sets of homologous chromosomes), Rht-B1b can make the final product or plant too short, along with decreasing seed and protein content, even more than in hexaploid wheat, because there is only one wild type Rht gene left. Whereas, in hexaploid wheat, there are two genes left when it carries Rht-B1b and Rht-D1b. In these cases, it might be contemplated to utilize an Rht-A or Rht-B1b-E529K intermediate height allele.

For Rht and triticale (a hybrid of wheat and rye), it is generally the same as hexaploid wheat, but in the case of triticale, it can only utilize or incorporate Rht-B1b, since it doesn't have the D genome.

On the other hand, for TB1 and hexaploid wheat, two knockouts of TB1 can increase tillering and biomass without negatively impacting seed yield. In these cases, one can combine two TB1 knockouts with Rht-B1b or Rht-D1b for additive and positive impact on tillering.

For TB1 and tetraploid wheat, one knockout dose is likely a better option for maintaining seed yield, while still increasing biomass. If the yield is being considered for hay alone, then two TB1 knockouts would work. In contemplated embodiments, one can combine these TB1 knockouts with Rht-B1b for maximum tillering effects, but TB1 mutations would likely be suitable alone, especially for seeds and plants being utilized in arid environments. In contemplated embodiments, one would utilize intermediate height Rht-A or Rht-B1b-E529K alleles combined with a single TB1 gene knockout.

For TB1 and triticale, it is generally the same as hexaploid wheat, but in the case of triticale, it can only utilize or incorporate Rht-B1b, since it doesn't have the D genome. Rht and TB1 mutations can be found in the R genome for triticale, much in the same way that those mutations exist in the D genome in wheat. In the case of triticale varieties, contemplated embodiments would include finding gene knockouts in the A, B, and R genomes for TB1, so that they could be utilized in both semi-dwarf (Rht-B1b) or tall (Rht-B1a) triticale varieties.

### EXAMPLES

### Example 1: TB1 Research Study

We identified TB1 mutations in both durum and spring wheat and completed field studies in 2019 and 2020 for our durum populations. Because of the similarity of durum and hexaploid wheat, our durum results are highly predictive of results that would be obtained by characterizing TB1 mutations in spring and winter wheat and in fact all wheat relative species. In durum, through crossing we obtained variant combinations for TB1 in both the A and B genomes (combos: WT-A/WT-B, WTA/mut-B, mut-A/WT-B, mut-A/mut-B). Testing in durum clearly demonstrates that this approach leads to significant increases in plant biomass and tiller number. Furthermore, while biomass and tiller number are significantly improved in our durum populations, seed yield was either unchanged, or only slightly down, while protein content was unchanged or increased. This is surprising and it may be specific to the individual alleles we have tested which both contain stop mutations very near the Carboxy terminus of the TB1 protein. This is an ideal trait to exploit in the development of dual-purpose forage winter wheat or increased protein content durum or hexaploid wheat.

We backcrossed the TB1 alleles of interest into durum varieties and we also tested this in hexaploid wheat. To that end, we have identified TB1 mutations from the A, B, and D genomes in hexaploid wheat. We have already intercrossed these alleles in spring wheat and have genotypes representing single, double, and triple TB1 variant combinations and have begun integrating them into two popular Montana spring wheat varieties. We have also begun to integrate the TB-A1 and TB-D1 null alleles into two winter wheat forage cultivars. Dual purpose forage wheats and other wheat relative species used as forage (such as triticale) are the most obvious application for this technology.

### Results: Durum

### TB1 Null Mutations Increase Tiller Number and Biomass:

Seed containing TB1 null mutations in both the A and B genome in the durum background Kronos was obtained from the TILLING database (www.wheat-tilling.com) (Krasileva et al., 2017) and planted in the greenhouse. Once plants reached anthesis, crosses were made to combine the A and B mutations in durum. This entailed the following crosses: TB-A1mut944 x TB-B1mut2226 and TB-A1mut944 x TB-B1mut562. Crosses were successful, and seed was advanced to the F2 generation at which point >200 plants were genotyped and segregated into four homozygous categories depending upon TB1 allele presence in which plants contained wildtype and mutant combinations for both A and B homeologs (TB-A1a/B1a, TB-A1mut/B1a, TB-A1a/B1mut; TB-A1mut/B1mut).

To assess the impact of the TB1 null mutations on tillering and biomass F2:3 homozygous segregants for each genotypic class were then grown in replicated spaced rows in the field under irrigation in 2019 (Bozeman, MT). F2:4 plants were then also grown in 2020 under irrigated (Bozeman, MT) and dryland conditions (Moccasin, MT). Data from the three location years was combined and significant changes were observed in tiller number and spikelet number per head resulting from the presence of the TB1 null allele in both crosses. Presence of a TB1 null allele (singly or in combination) led to increased tiller numbers for both crosses and a decrease in spikelets per head (Table 1 and 3). As expected, there was a negative correlation between tiller number and number of spikelets. The cross Kronos944 x Kronos562 was of particular interest as increased tiller number translated into significantly increased biomass for the mut/mut and wt/mut genotypes where biomass was increased up to 15% in lines carrying both TB1 null alleles (Table 2).

There was also a significant decrease in harvest index in these genotypes indicating an increase in vegetative tissue compared to seed production (Table 2). The changes observed in mutant classes for the Kronos944 x Kronos2226 cross for biomass and harvest index were similar (Table 4). We also measured seed protein and seed size and surprisingly found increased seed protein content in genotypes carrying one or two TB1 mutations in the Kronos944 x Kronos562 cross while individual seed size and yield was not changed (Table 2). Mutant carrying lines also showed a trend in decreased seeds per head, which correlates with the decreased spikelet number (Table 2). The increase in protein content in mutants was not observed in the Kronos944 x Kronos2226 cross (Table 4). Like the Kronos562 cross though there was no significant difference in individual seed weight or yield and the single and double mutants had less seeds per head, significantly so for the double mutant (Table 4).

Interestingly, TB1 null lines Kronos944 (TB-A1-W339Stop) and Kronos562 (TB-B1-W341Stop) carry equivalent mutation that occurs near the Carboxy terminus of the protein. The TB-B1 coding sequence is two amino acids longer which is why the amino acid numbering is different for that tryptophan (W). These alleles may be key to the results we obtained since we did not see an increase in protein content or biomass in our other cross (Kronos944 x Kronos2226 (TB-B1-Q142stop)).

Our results demonstrate that TB1 null alleles can be used to increase plant biomass and increase the value of wheat and relative species as forage or for hay. That would include spring wheat, winter wheat, durum, triticale, and other wheat relative species. Our results also demonstrate that the specific TB1 null alleles in Kronos944 and Kronos562 should be patented and would be valuable as a method and materials to increase total biomass under varying environments in wheat by up to 15% while also increasing seed protein content by at least 5%.

TABLE 1
Plant Characteristics of Kronos944 (TB-A1 W339stop) ×
Kronos562 (TB-B1 W341stop) over three location years.

| | | | | Leaf | Leaf | Plant | |
|---|---|---|---|---|---|---|---|
| | | | Productive | Length | width | Height | |
| ID^{a} | n | Tillers | Heads | mm | mm | cm | Spikelets |
| WT-A/WT-B | 48 | 14.9 | 12.0 | 20.2 | 1.5 | 69.7 | 15.0 |
| SE | | 0.4 | 0.4 | 0.2 | 0.0 | 0.8 | 0.1 |
| Mut-A/Mut-B | 27 | 16.7 | 13.4 | 19.4 | 1.5 | 68.4 | 14.5 |
| SE | | 0.7 | 0.7 | 0.3 | 0.0 | 1.2 | 0.1 |
| P-value | | 0.013 | 0.034 | 0.016 | 0.372 | 0.176 | 0.005 |
| Mut-A/WT-B | 24 | 15.8 | 12.5 | 19.4 | 1.5 | 69.3 | 14.6 |
| SE | | 0.7 | 0.7 | 0.3 | 0.0 | 1.2 | 0.2 |
| P-value | | 0.116 | 0.273 | 0.021 | 0.022 | 0.398 | 0.034 |
| WT-A/Mut-B | 54 | 15.9 | 12.6 | 20.2 | 1.5 | 69.8 | 14.9 |
| SE | | 0.5 | 0.5 | 0.2 | 0.0 | 0.8 | 0.1 |
| P-value | | 0.064 | 0.163 | 0.463 | 0.417 | 0.487 | 0.325 |

^{a}P-values are from Students t-tests where each genotypic class was compared to the WT-A/WT-B genotype. Values presented are on a single plant basis.
V

TABLE 2
Agronomic and Seed Characteristics of Kronos944 (TB-A1
W339stop) × Kronos562 (TB-B1 W341stop) over three
location years.

| | | | | Individual | | | |
|---|---|---|---|---|---|---|---|
| | | | | Seed | | | |
| | | Biomass^{b} | Yield^{b} | Weight^{c} | Protein^{d} | | Harvest |
| ID^{a} | n | g | g | mg | % | Seed/Head | index |
| WT-A/WT-B | 48 | 27.2 | 23.8 | 49.4 | 14.7 | 56.9 | 46.3 |
| SE | | 0.8 | 0.9 | 1.0 | 0.1 | 2.1 | 1.3 |
| Mut-A/Mut-B | 27 | 31.4 | 21.9 | 51.1 | 15.2 | 51.5 | 41.7 |
| SE | | 2.3 | 1.3 | 0.9 | 0.2 | 2.9 | 2.0 |
| P-value | | 0.020 | 0.107 | 0.128 | 0.020 | 0.066 | 0.025 |
| Mut-A/WT-B | 24 | 27.3 | 23.8 | 49.5 | 15.2 | 55.0 | 46.5 |
| SE | | 1.1 | 1.1 | 1.2 | 0.2 | 2.9 | 1.5 |
| P-value | | 0.475 | 0.497 | 0.496 | 0.014 | 0.304 | 0.462 |
| WT-A/Mut-B | 54 | 29.8 | 23.5 | 50.0 | 14.8 | 55.1 | 43.6 |
| SE | | 1.0 | 1.0 | 0.9 | 0.2 | 2.3 | 1.1 |
| P-value | | 0.020 | 0.401 | 0.342 | 0.333 | 0.281 | 0.059 |

^{a}P-values are from Students t-tests where each genotypic class was compared to the WT-A/WT-B genotype.
^{b}Biomass and yield data presented is on a single plant basis.
^{b}Seed weight is the average of 300 seeds.
^{c}Protein determined by NIR.

TABLE 3
Plant Characteristics of Kronos944 (TB-A1-W339stop) ×
Kronos2226 (TB-B1-Q142stop) over three location years.

| | | | | Leaf | Leaf | Plant | |
|---|---|---|---|---|---|---|---|
| | | | Productive | Length | width | Height | |
| ID^{a} | n | Tillers | Heads | mm | mm | cm | Spikelets |
| WT-A/WT-B | 33 | 14.4 | 11.4 | 18.3 | 1.5 | 62.2 | 14.9 |
| SE | | 0.5 | 0.5 | 0.3 | 0.0 | 1.0 | 0.1 |
| Mut-A/Mut-B | 30 | 16.7 | 13.4 | 16.9 | 1.4 | 65.1 | 14.4 |
| SE | | 0.8 | 0.7 | 0.2 | 0.0 | 1.0 | 0.2 |
| P-value | | 0.004 | 0.007 | 0.000 | 0.092 | 0.017 | 0.007 |
| Mut-A/WT-B | 33 | 15.1 | 12.2 | 17.3 | 1.4 | 64.7 | 14.8 |
| SE | | 0.5 | 0.5 | 0.2 | 0.0 | 0.9 | 0.1 |
| P-value | | 0.214 | 0.174 | 0.002 | 0.331 | 0.036 | 0.245 |
| WT-A/Mut-B | 30 | 15.2 | 12.2 | 17.9 | 1.4 | 62.5 | 14.6 |
| SE | | 0.6 | 0.6 | 0.3 | 0.0 | 0.8 | 0.1 |
| P-value | | 0.181 | 0.165 | 0.149 | 0.196 | 0.409 | 0.046 |

^{a}P-values are from Students t-tests where each genotypic class was compared to the WT-A/WT-B genotype.

TABLE 4
Agronomic and Seed Characteristics of Kronos944 (TB-A1-
W339stop) × Kronos2226 (TB-B1-Q142stop) over
three location years.

| | | | | Individual | | | |
|---|---|---|---|---|---|---|---|
| | | | | Seed | | | |
| | | Biomass^{b} | Yield^{b} | Weight^{c} | Protein^{d} | | Harvest |
| ID^{a} | n | g | g | mg | % | Seed/Head | index |
| WT-A/WT-B | 33 | 20.4 | 19.9 | 42.5 | 14.3 | 60.6 | 48.4 |
| SE | | 1.1 | 1.4 | 0.9 | 0.1 | 3.1 | 1.7 |
| Mut-A/Mut-B | 30 | 22.6 | 20.5 | 41.9 | 14.2 | 51.7 | 47.7 |
| SE | | 1.4 | 1.3 | 1.2 | 0.3 | 2.6 | 1.9 |
| P-value | | 0.216 | 0.184 | 0.273 | 0.193 | 0.012 | 0.258 |
| Mut-A/WT-B | 33 | 20.8 | 20.5 | 42.7 | 14.4 | 56.0 | 48.7 |
| SE | | 1.0 | 1.3 | 0.7 | 0.2 | 2.3 | 2.1 |
| P-value | | 0.386 | 0.362 | 0.425 | 0.351 | 0.124 | 0.454 |
| WT-A/Mut-B | 30 | 21.3 | 20.7 | 44.3 | 14.4 | 56.1 | 48.7 |
| SE | | 1.0 | 1.2 | 0.8 | 0.2 | 2.6 | 1.8 |
| P-value | | 0.285 | 0.330 | 0.065 | 0.250 | 0.141 | 0.461 |

^{a}P-values are from Students t-tests where each genotypic class was compared to the WT-A/WT-B genotype.
^{b}Biomass and yield data presented is on a single plant basis.
^{b}Seed weight is the average of 300 seeds.
^{c}Protein determined by NIR.

### Example 2: Rht Research Study

The goal was to identify Rht mutations that reduce height but not as much as Rht-B1b or Rht-D1b while still increasing yield. Thus far, we have the most complete results in durum wheat. In fact, partially reducing plant height in durum likely has the greatest value if the new Rht alleles decrease plant height while increasing yield without decreasing seed protein content.

We mutagenized a standard height durum variety, advanced it by single seed descent to homozygosity, then screened for Rht-A1 mutations. Rht-A1 mutations were most desirable because then they could be used either alone or in combination with an Rht-B1b allele. In so doing, it would be possible to create a range of plant heights. We had two primary mutations of interest, Rht-A1-S50T and Rht-A1-L358F. These were crossed with both standard height (Rht-B1a) and semi-dwarf (Rht-B1b) varieties. We then screened F2 plants to identify those that were homozygous for individual genotypes and then measured various plant traits.

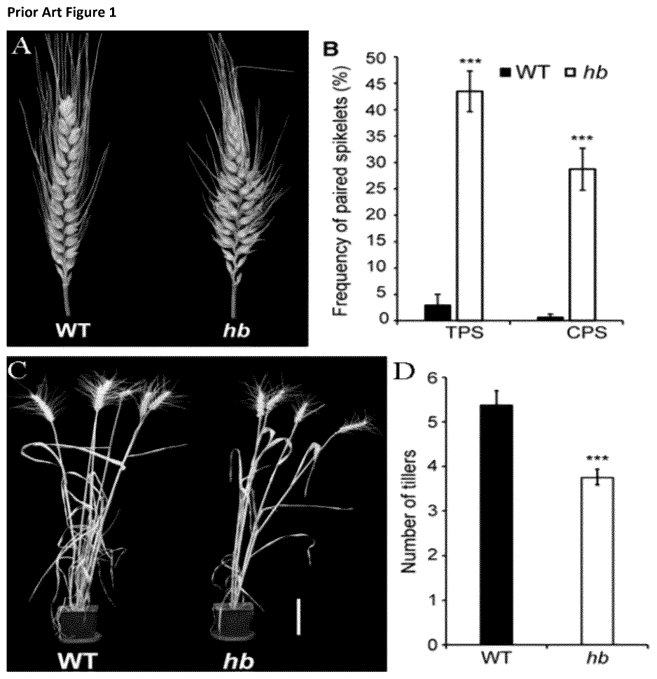

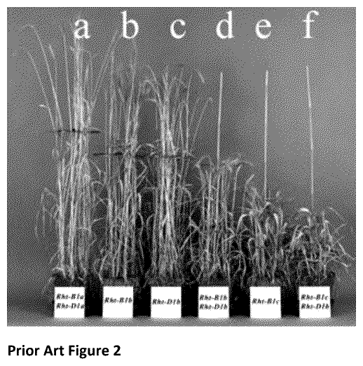

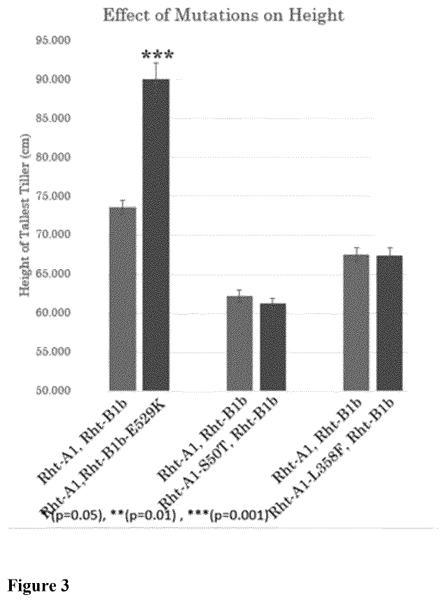

From crosses to the semi-dwarf durum (MT112219) we found that S50T and L358F did not reduce plant height while the control line Rht-B1b-E529K increased plant height (FIG. 3). While Rht-A1S50T did not change plant height, yield was increased on a per plant basis by ˜10%.

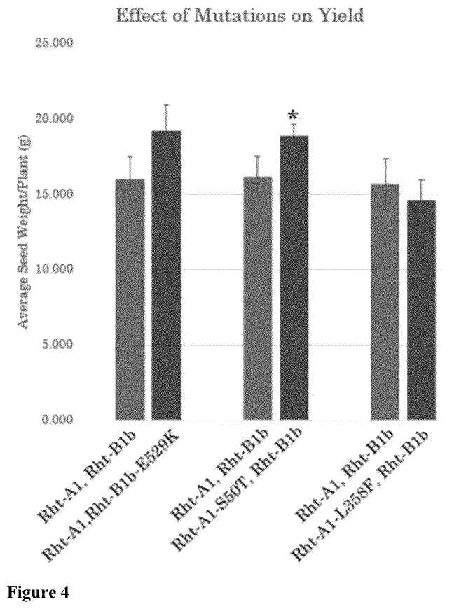

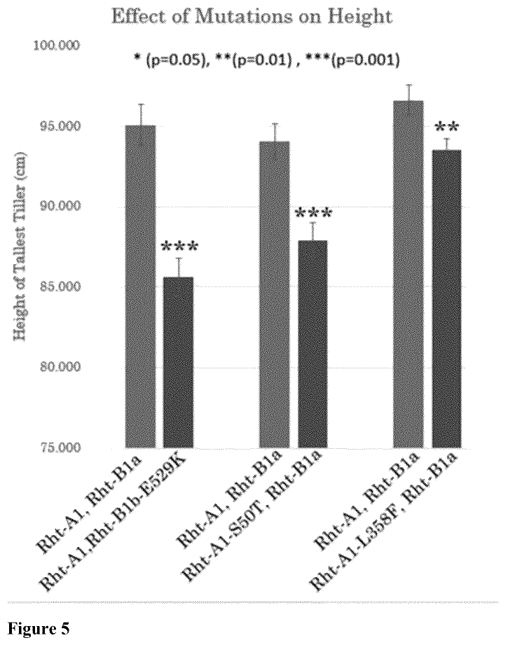

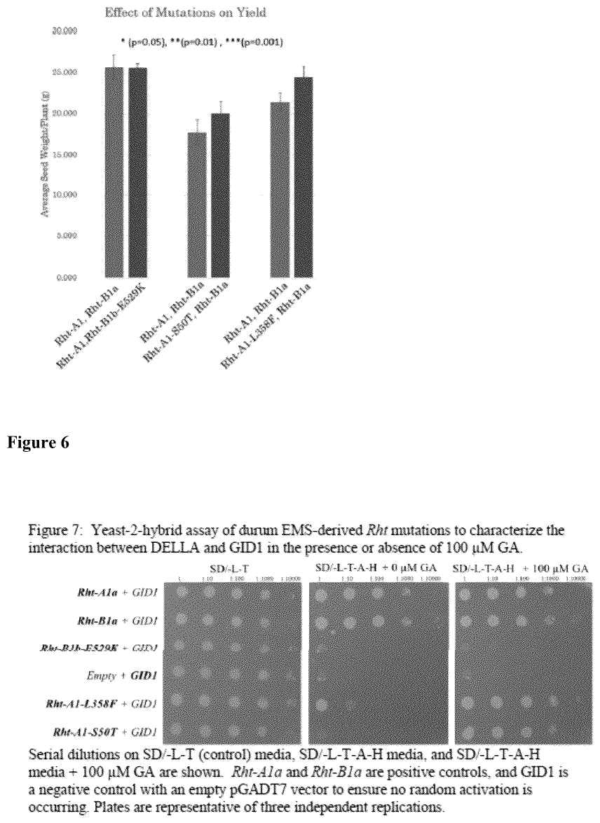

To test whether the new Rht alleles affect plant height and yield in the absence of Rht-B1b, we also crossed S50T and L358F to a standard height durum. We found that both S50T and L358F reduced plant height and to specific levels not possible with current Rht tall or semi-dwarf alleles (FIGS. 4-6). (The Rht-B1b-E529 allele was discovered and tested by UC Davis who published a paper demonstrating the impact of this allele on plant height. The UC Davis researchers did not report any other alleles, nor did they report yield data. S50T and L358F also had a distinct trend toward increased yield.

To test whether the new Rht alleles affect seed protein content in the absence of Rht-B1b, we measured seed protein content and found that S50T had a trend of increased protein content while L358F increased seed protein content from 16.5 to 17.4%. This is the opposite of the impact of Rht-B1b as shown in multiple previous studies (Table 5).

Plant traits measured on progeny lines containing Rht-A1/Rht-B1b-E529K, Rht-A1-S50T/Rht-B1a, or Rht-A1-S358F/Rht-B1a. Rht-A1-L358F decreases height, increases tillers, exhibits distinct trends toward increased productive heads and yield while increasing seed protein content. Rht-A1-S50T decreased plant height and had distinct trend of increased seed weight without reducing seed protein content.

Both Rht-A1-L358F and Rht-A1-S50T are likely to have distinct advantages in durum production systems. Similarly, Rht-A1 missense or nonsense alleles in the same Rht region as Rht-A1-L358F and Rht-A1-S50T would be expected to create similar beneficial phenotypes in hexaploid wheat of increased productivity without reduced protein content.

TABLE 5

| | | | | | | | |
|---|---|---|---|---|---|---|---|
| | | | Flag | Flag | | | |
| | | Productive | Leaf | Leaf | | | Ind. See |
| Height | Tillers | Heads | Length | Width | Yield | Protein | Weight |
| (cm) | (#) | (#) | (cm) | (cm) | (g) | (%) | (g) |

MT16005*2/Divide 169

| | | | | | | | | |
|---|---|---|---|---|---|---|---|---|
| Rht-A1, | 96.62 | 15.77 | 3.92 | 1.64 | 21.96 | 21.37 | 16.46 | 0.05 |
| Rht-B1 | | | | | | | | |
| (n = 13) | | | | | | | | |
| Std | 3.36 | 2.65 | 1.04 | 0.10 | 1.70 | 3.88 | 0.51 | 0.00 |
| Se | 0.93 | 0.74 | 0.29 | 0.03 | 0.47 | 1.08 | 0.14 | 0.00 |
| Rht-A1- | 93.52 | 18.43 | 5.67 | 1.70 | 22.23 | 24.44 | 17.38 | 0.05 |
| L358F, | | | | | | | | |
| Rht-B1 | | | | | | | | |
| (n = 21) | | | | | | | | |
| Std | 3.03 | 3.25 | 6.44 | 0.13 | 1.93 | 6.13 | 0.40 | 0.00 |
| Se | 0.66 | 0.71 | 1.40 | 0.03 | 0.42 | 1.34 | 0.09 | 0.00 |
| Ttest P value | 0.00 | 0.01 | 0.17 | 0.10 | 0.34 | 0.06 | 0.01 | 0.49 |

MT16005*2/Divide 4

| | | | | | | | | |
|---|---|---|---|---|---|---|---|---|
| Rht-A1, | 94.80 | 15.40 | 7.05 | 1.59 | 21.59 | 19.70 | 17.37 | 0.05 |
| Rht-B1 | | | | | | | | |
| (n = 13) | | | | | | | | |
| Std | 4.54 | 3.78 | 1.73 | 0.18 | 1.18 | 5.49 | 0.26 | 0.00 |
| Se | 0.95 | 0.79 | 0.36 | 0.04 | 0.25 | 1.15 | 0.05 | 0.00 |
| Rht-A1- | 88.48 | 14.36 | 6.41 | 1.60 | 21.75 | 21.31 | 17.55 | 0.05 |
| L358F, | | | | | | | | |
| Rht-B1 | | | | | | | | |
| (n = 21) | | | | | | | | |
| Std | 5.11 | 3.32 | 1.94 | 0.14 | 1.82 | 6.47 | 0.41 | 0.00 |
| Se | 1.00 | 0.65 | 0.38 | 0.03 | 0.36 | 1.27 | 0.08 | 0.00 |
| Ttest P value | 0.00 | 0.17 | 0.13 | 0.39 | 0.37 | 0.19 | 0.22 | 0.22 |

To identify alleles useful to improve hexaploid wheat yield without the negatives of reduced seed size and protein content or Rht-B1b or Rht-D1b we screened an Alpowa (soft white spring hexaploid wheat variety) EMS population for mutations in the 5′ portion of Rht. The S50T and L358F alleles have SIFT values of −2.1 and −3.8 respectively, so we have selected a set of lines to test (Table 2). They will be tested as we did for the S50T and L358F alleles, by crossing to both standard height (Rht-A1a, Rht-B1a) varieties and to semi-dwarf varieties (Rht-A1a, Rht-B1b or Rht-D1b). The impact of each unique allele upon wheat agronomic traits will be measured and it is anticipated that one or more of the alleles shown in Table 6 will increase yield without decreasing individual seed size and protein content.

TABLE 6
Rht-A1 mutations discovered by screening ~1000 Alpowa EMS lines. The
Divide mutations described above are shown for reference. We have selected
a set of Rht-A1 missense (and one nonsense) alleles for further testing.

| EMS | Rht | | Codon | Codon | Aa | Aa | | | | |
|---|---|---|---|---|---|---|---|---|---|---|
| Line | bp | AA# | orig | new | orig | new | Mutation | Type | SIFT | |
| Div 4 | 149 | 50 | tcc | ttc | S | T | S50T | Missense | −2.123 | Neutral |
| Div 169 | 1072 | 358 | ctc | ttc | L | F | L358F | Missense | −3.821 | Deleterious |
| 1199 | 293 | 98 | tcc | ttc | S | F | S98F | Missense | −2.861 | Deleterious |
| 1305 | 281 | 94 | ccc | ctc | P | L | P94L | Missense | −8.812 | Deleterious |
| 1390 | 293 | 98 | tcc | ttc | S | F | S98F | Missense | −2.861 | Deleterious |
| 1461 | 187 | 63 | gag | aag | E | K | E63K | Missense | −3.282 | Deleterious |
| 1536 | 7 | 3 | cgc | tgc | R | C | R3C | Missense | −4.903 | Deleterious |
| 1635 | 284 | 95 | acc | atc | T | I | T95I | Missense | −2.840 | Deleterious |
| 1700 | 187 | 63 | gag | aag | E | K | E63K | Missense | −3.282 | Deleterious |
| 1793 | 272 | 91 | cac | cgc | H | R | H91R | Missense | −7.058 | Deleterious |
| 1878 | 166 | 56 | gcg | acg | A | T | A56T | Missense | −3.266 | Deleterious |
| 1916 | 8 | 3 | cgc | cac | R | H | R3H | Missense | −2.769 | Deleterious |
| 1930 | 159 | 53 | gcg | gtg | A | V | A53V | Missense | −2.741 | Deleterious |
| 2141 | 157 | 53 | gcg | acg | A | T | A53T | Missense | −2.725 | Deleterious |
| 1158 | 6 | 2 | cag | tag | Q | * | Q2* | Nonsense | Assuming | Deleterious |

New alleles of Rht have been identified that confer height intermediate between standard height and semi-dwarf height in both durum and hexaploid (spring and winter) wheat. These alleles may prove particularly useful in environments in which standard semi-dwarf wheat is too tall (as in irrigated production in AZ and CA) or too short (as in dryland production in the northern US). Rht semi-dwarf alleles also increase the number of productive tillers per plant and using a new allele that confers intermediate height is likely to have a reduced number of productive tillers. Combining mutations in a second gene that increases productive tillers would enable stable increased yield while still retaining the desired height. The second gene is TB1. TB1 null mutations increase wheat tillering and plant biomass and so would be ideal to combine with an Rht allele conferring intermediate plant height.

As part of this work, we have identified a suite of Reduced Height (Rht) and Teosinte Branched 1 (TB1) gene mutations that can be applied individually and/or in combination to create optimal height and tillering balances without sacrificing yield and quality. These mutant Rht and TB1 alleles have applications in hexaploid wheat for grain producing, forage, and dual-purpose winter forage/grain varieties. For example, the Rht alleles can increase hexaploid wheat tillering without dragging down protein for grain producing varieties. However, there is great potential to improve forage and dual-purpose varieties. The herein described TB1 knockout increased durum biomass up to 18%. Similar increases are anticipated in hexaploid wheat. The result would be higher forage yields for forage varieties, and more grazeable biomass without a reduction in seed protein levels for dual-purpose winter wheats. For grain producing varieties, a combination of one TB1 knockout and one Rht allele bred into a full height or semi-dwarf wheat is anticipated to provide the capability to optimize phenotype features to meet particularized agricultural needs.

Because Rht and TB1 affect different mechanisms within plants, the alleles of each can be utilized in various combinations to fine tune plant height and tillering for the purposes of maximizing economic return. Most current options involve tradeoffs between yield and seed protein. This current invention has demonstrated the ability to increase yield without sacrificing protein quality. In fact, in many instances, protein levels are not only maintained but actually increased.

There are several benefits to utilizing a TB1 gene mutation, a RHT gene mutation or a combination thereof, including: increase grain and/or forage yield, while maintaining or even increasing seed protein; not genetically modified (Non-GMO) solutions to optimize variety performance; Rht and TB1 discoveries can be stacked to fine tune varieties; ability to product intermediate height durum varieties that are optimized for prime growing regions in North America, including Montana, North Dakota, and Canada; potential to improve forage yields by at least 18% without sacrificing seed yield or quality in dual purpose varieties.

### Example 3: Second Rht Research Study

The impacts of other mutant Rht alleles including Rht-B1c, Rht-B1d, Rht-B1e, Rht-D1c, Rht-D1d, Rht-8, Rht-3 (the Tom Thumb dwarfing gene), and Rht-10 have also been thoroughly examined in hexaploid wheat, but no Rht-A mutations have been found to have a height reducing effect on plants (Flintham et al. 1997, Gale and Youssefian 1985, Pearce et al. 2011). However, due to durum's tetraploid genome, phenotypic effects in hexaploid wheat may be different in tetraploid durum wheat. Rht mutant alleles are dosage-dependent, so in tetraploid wheat the effect of an Rht mutation in one genome out of two could be stronger than the effect of a single-genome mutation and two wild type genomes. In hexaploid wheat, Rht-A is expressed in the stem at similar levels as Rht-B (Pearce et al. 2011); therefore, we proposed that Rht-A mutations in durum wheat may also have a height-suppressing effect. Less-severe mutations in either Rht-A1 or Rht-B1 could create a plant phenotype intermediate between the current Rht-B1b and the wild type Rht-B1a.

To test this hypothesis, an EMS-mutagenized population of durum wheat was screened for mutations in Rht-A. Lines containing missense mutations with low PROVEAN scores and reduced plant height were identified as sources for variation. These lines were crossed with both a standard-height and a semi-dwarf parent to create segregating populations that were then grown in field trials to evaluate for differences in height. Coleoptile length differences were also evaluated. Additionally, in-vitro testing of the alleles was conducted with a yeast-2-hybrid assay.

Along with the novel Rht-A mutations tested, the previously identified and described Rht-B1b-E529K mutation was included in each of the described experiments to confirm its effects in Montana-adapted varieties. This missense mutation confers an intermediate-height plant by partially suppressing the semi-dwarf phenotype typically associated with the Rht-B1b allele (Mo et al. 2018). Rht-B1b-E529K was found to increase plant height by 21% and is associated with increases in coleoptile length, seedling shoot length, and stem internode length, all relative to Rht-B1b (Mo et al. 2018).

### Materials and Methods

### Identification and Selection of Mutant Lines

To analyze variation caused by Rht-A1 mutations in the presence and absence of Rht-B1b, an EMS (ethyl methanesulfonate) population was created in the standard height durum variety “Divide” (PI 642021). It was created by mutagenizing 10,000 seeds using the method described by Slade et al. (2005) and modified by Feiz et al. (2009). The population was then advanced through single seed descent through the M3 generation. One thousand, nine hundred M4 head rows were grown in the field and plants were bulk harvested for M3:5 seed. The head rows were measured for plant height, and the 192 shortest rows were screened for mutations in the entire coding sequence of the Rht-A gene. Seven missense and four silent mutations were identified, and the two lines containing missense mutations with the lowest PROVEAN scores were chosen for this project: Rht-A1-S50F and Rht-A1-L358F (Table 7). The “Kronos” TILLING mutant (UC Davis, Tsai et al. 2011, Krasileva et al. 2017) allele Rht-B1b-E529K was also included to analyze and compare its effect in Montana adapted lines to the observations demonstrated in Mo et al. (2018).

TABLE 7
Summary of Rht-1 mutations including location
and predicted effect on protein function.

| | DNA change (base | | | |
|---|---|---|---|---|
| | pairs from start | Original | New | PROVEAN |
| Allele | codon) | Codon | Codon | score^{a} |
| Rht-A1-S50F | 149 | TCC | TTC | −2.12 |
| Rht-A1-L358F | 1072 | CTC | TTC | −3.82 |
| Rht-B1b-E529K | 1585 | GAG | AAG | −3.49b |

^{a}PROVEAN (Protein Variation Effect Analyzer) score used to predict effect of a missense amino acid change: A score of −2.5 for single amino acid substitutions is predicted to be deleterious 80% of the time, with higher scores having a deleterious effect less often and lower scores more often.
^{b}Rht-B1b-E529K contains both a stop mutation at Q64* and a missense mutation downstream at the position shown above. The PROVEAN score for this situation cannot be accurately calculated; the score shown is the missense mutation alone.

Creation of BC₁F₃ Lines

Recurrent, Montana-adapted parents for backcrossing include “MTD16005” (full height durum, fixed for Rht-B1a) and “MT112219” (semi-dwarf durum, fixed for Rht-B1b). F1 crosses were made between each of the three mutant lines and both parents, for a total of 6 F1 crosses. The first backcross was made, then plants allowed to self for BC1F2 seed. DNA was extracted from the BC₁F₂ plants, then PCR was performed using the nested approach described by Li et al. (2013) in which genome specific primers are used for initial segment amplification, then primers are used to amplify the region containing the Rht-A1-L50F and Rht-A1-L358F mutations. The primers from Mo et al. (2018) were used to detect the Rht-B1b-E529K mutation. PCR products were Sanger sequenced (GENEWIZ, Inc., Cambridge, MA, USA), then the sequence analyzed against reference accessions JF930277.1 (Rht-A1) and JF930278.1 (Rht-B1) (www.ncbi.nlm.nih.gov) using the DNASTAR SeqMan Pro software (DNASTAR Version 15.0.1.1, Madison, WI, USA). Lines were genotyped for Rht-A mutations, Rht-B1b-E529K, and Rht-B1b, and plants that were heterozygous for Rht-A mutations and Rht-B1b-E529K but fixed for Rht-B1a and Rht-B1b were kept. This allowed for selection only for the mutations of interest in future generations.

BC₁F₂ seed was grown in the 2019 field season as spaced plants, then genotyped as described above and only Rht homozygous plants were kept for measurement and harvest. Each single plant provided BC₁F_{2:3} seed for one row in the 2020 field season.

### Greenhouse Growing Conditions

All crossing occurred in the greenhouse, set at 22.2° C. in the daytime and 18.3° C. at night. Seeds were planted in Sunshine mix (Sungro Horticulture, Agawam, MA, USA) potting soil, 4 seeds per 20.3 cm pot. Plants were watered daily with a 100 ppm solution of 20-20-20 fertilizer using a Dosatron (Dosatron International, Inc., Clearwater, FL, USA) once plants reached the three-leaf stage. After flowering, the temperature was increased to 25° C. day and 21° C. night until harvest. A micronized sulfur solution (Bonide Products, Inc., Oriskany, NY, USA) at a rate of 4.38 g/l was applied to plants at the 2-3 leaf stage (Z 12-Z13 on the Zadoks growth scale for wheat) for powdery mildew and mite prevention, and Marathon (Imidacloprid 1%, OHP, Inc., Bluffton, SC, USA) at a rate of 2.7 g per pot was applied at the same growth stage for aphid control.

### Field Growing Conditions and Layout

One hundred BC₁F₂ plants from each of the six crosses were grown in 2019 under irrigated conditions at the Arthur H. Post Agronomy Farm near Bozeman, MT (latitude 45.67N, longitude 111.15W, elevation 1,455 m, soil type is Amsterdam silt loam). After testing soil, 185 kg per ha urea (46-0-0) was added. Seeds were hand planted to a depth of 2.5 cm on May 10, 2019. At harvest maturity, single plants were cut at ground level, weighed, and grain collected using a single-plant thresher the second week of September. From May 1st to August 31st, the research station received 18.6 cm of precipitation. The highest recorded air temperature was on July 23 at 32.3° C., while the lowest recorded air temperature was −0.56° C. on May 19 (NOAA Bozeman). Five cm irrigation water was applied using hand line sprinklers on July 1. A 1.1 l/ha application of Huskie (Bayer CropScience, Research Triangle PK, NC, USA) was applied for broadleaf weed control, and throughout the growing season weeds were rogued out by hand.

The plants were grown in 3-m rows with 30-cm spacing between adjacent rows. Within each row, seeds were sown 23 cm apart with 13 plants per row. There were 100 spaced plants of each of the 6 BC₁F₂ crosses planted. One hundred and twenty BC₁F₂-derived F3 lines (ten mutant lines and ten wild type lines from each of the 6 crosses) were grown in two replicates of a randomized complete block design under the above-described irrigated conditions at the Arthur H. Post Agronomy Farm in 2020.

Seeds were hand planted to a depth of 2.5 cm on April 28th. At physiological maturity, single plants were cut at ground level, weighed whole, and threshed with a single-plant thresher the first week of September. From May 1 to August 31, the research center received 14.3 cm of precipitation. The highest recorded air temperature was 34.4° C. on both August 17 and 18, while the lowest recorded air temperature was −0.6° C. on May 8 and 11 (NOAA Bozeman). The plants were grown in 1.5 m rows with 30 cm spacing between rows. Within a row, seeds were sown 15 cm apart with 8 plants per row. The trial was fertilized with 40 kg per ha urea (185 kg N per ha), and weed control consisted of 0.06 liter/hectare of Affinity Broadspec (FMC, Philadelphia, PA, USA), 0.58 liter/hectare MCPA Ester 4 (Albaugh, LLC, Ankeny, IA, USA), 1.2 liter/hectare Discover NG (Syngenta Crop Protection, LLC, Greensboro, NC, USA), and 0.29 liter/hectare Propi-Star EC (Albaugh, LLC, Ankeny, IA, USA).

The same trial was planted on May 11, 2020 at the Central Agricultural Research Center near Moccasin, MT (latitude 47.06N, longitude 109.96W, elevation 1,297 m, soil type is Danvers-Judith clay loam). This was a dryland site, and as such did not receive any additional moisture from irrigation. Seeds were planted to a depth of 2.5 cm and harvested the third week of August. From May 1 to August 31, the research center received 16.03 cm of precipitation. The highest recorded air temperature was on August 17 at 33.9° C., while the lowest recorded air temperature was −1.1° C. on May 12 (NOAA Stanford). The plot was fertilized with 67.3 kg/ha ESN slow-release nitrogen fertilizer (44-0-0, Nutrien, Saskatoon, SK, CA). Vendetta (Wilbur-Ellis Agribusiness, Aurora, CO, USA) was applied on May 27th for broadleaf weed control.

### Plant Measurements

Plant height was measured near physiological maturity, after growth had ceased, by determining the distance from the soil surface to the terminal spikelet of the tallest tiller. Tiller number was counted at the base of these plants, regardless of whether the tiller produced a head. Flag leaf length of the tallest tiller was measured from culm to tip, and width at the widest point of the leaf. The number of productive heads included only heads that formed seed.

Seed protein content was analyzed with the LECO-FP 528 (LECO Co., St. Joseph, MI, USA; AACCI Method 46-30.01) and individual seed weight was calculated by hand, using a Contador seed counter (Pfeuffer, Kitzingen, Bavaria, Germany) on a sample of 100 seeds. In 2019, single plants were measured after genotyping, while in the 2020 five representative plants per row were measured and averaged together.

### GA Responsiveness Assay: Coleoptile Length

To evaluate the impact of the Rht-1 mutations on coleoptile length and responsiveness to GA, we used the ‘cigar roll’ technique described by Bai et al. (2013). BC₁F₄ seeds were surface sterilized and soaked in water for 24 hours. One roll consisted of 10 seeds from a single BC1F4 line placed approximately 2 cm apart on 46×31 cm germination paper (Anchor Paper Co., St. Paul, MN, USA). The germination paper was pre-soaked in water or in a 100 μM GA3 solution.

The seeds were sandwiched by folding over the germination paper, and then rolled to a diameter of 2 cm. The BC₁F₄ lines consisted of ten mutant and wild type lines from each of the six crosses, for a total of 120 rolls in one replication of the experiment. Two separate replications were performed. The rolls were randomized and placed upright in test tube racks, then placed in tubs which submerged the bottom two cm of the rolls in either water or 100 μM GA3 solution. The tubs were then placed in a dark growth chamber at 18° C. Coleoptile length, measured as the distance from the edge of the seed to the end of the coleoptile, was recorded after 10 days.

### GA Responsiveness Assay: GID1 Interaction

Yeast-2-hybrid assays were used to analyze protein-protein interaction. The bait plasmid (pGBKT7) contained GID1, while the prey plasmids (pGADb) each contained one Rht allele. Genes were synthesized and cloned into vectors using GENEWIZ Gene Synthesis Services (GENEWIZ, Plainfield, NJ, USA). Bait and prey vectors were co-transformed into the Saccharomyces cerevisae strain Y2HGold using the Yeastmaker Yeast Transformation System 2 (Clontech, Mountain View, CA, USA) and the Matchmaker Gold Yeast Two-Hybrid System (Takara Bio Inc., Mountain View, CA, USA). Colonies were grown on selectable SD media lacking leucine and threonine (SD/-L-T) for 4-5 days to select for successful co-transformation. SD/-L-T broth was then inoculated with single colonies and grown overnight. Each line to be tested was brought to an internally consistent OD600 reading of ˜0.8, then serial dilutions of 1:10, 1:100, 1:1000, and 1:10000 were prepared. Dilutions, along with full strength solution, of each line were plated on SD media lacking leucine, threonine, adenine, and histidine, (SD/-L-T-A-H) containing 100 μM GA and the same media containing no GA. Growth of each Rht mutant line was compared to Rht-A1a and Rht-B1a positive controls and a GID1 negative control consisting of the pGBKT7-GID1 vector co-transformed with an empty pGAD vector.

### Statistical Analysis

Comparisons between the mutant and wild type sister line means were made using a one-way analysis of variance (ANOVA) in R (R Foundation for Statistical Computing, Vienna, Austria) for the 2019 spaced plant trial. Data were combined over the two locations for the 2020 trials first computing the mean over the two replications for each line. Then a mixed linear model was fitted in R using the Imer4 package in R (Bates et al., 2015) where the model included location, genotype class, lines within genotype, and location x genotype class. Individual lines within genotype class were considered random and other factors were fixed. Data for the coleoptile length trial were analyzed as a randomized complete block design where the model included replication, genotype class and lines within genotype class using the Imer4 package in R where lines within genotype class were considered fixed. Differences between mutant and wild type means were determined from an F ratio from the ANOVA.

### Results

### Field 2019 Trial

The effect of durum Rht mutations in BC1F2 sister lines under field conditions was tested with a spaced, single-plant trial (Tables 8a and 8b). In the semi-dwarf Rht-B1b background, only the Rht-B1b-E529K mutation caused significant differences, reflected as an increase in plant height of 22% (16.5 cm). In the full height Rht-B1a background, the Rht-B1b-E529K mutation decreased height by 11% (10.1 cm). Both Rht-A1-S50T and Rht-A1-L358F also caused a decrease in plant height, the former by 7% (6.2 cm) and the latter by 3% (3.1 cm). Rht-A1-L358F also increased tillers by 17% (2.7 tillers) and protein by 5.5% (0.9%), relative to the wild type plants.

TABLE 8A
The impact of Rht mutations on genotype class mean values
for agronomic traits from 2019 Bozeman field trials.

| Recurrent | | | Plant | | Productive | Flag Leaf | Flag Leaf |
|---|---|---|---|---|---|---|---|
| Parent Rht | Rht-1 | | Height^{b} | Tillers^{b} | Heads^{b} | Length^{b} | Width^{b} |
| Allele | allele | n^{a} | Cm | No./plant | No./plant | Cm | cm |
| Rht-B1b | Rht-B1b-E529K | 22 | 90.0 ± 2.07*** | 11.8 ± 0.71 | 8.23 ± 0.73 | 18.5 ± 0.49 | 1.59 ± 0.02 |
| | Rht-B1b | 16 | 73.5 ± 0.94 | 12.0 ± 0.72 | 7.38 ± 0.76 | 18.4 ± 0.52 | 1.52 ± 0.00 |
| | Rht-A1-S50F | 23 | 61.3 ± 0.67 | 15.7 ± 0.76 | 7.09 ± 0.34 | 17.5 ± 0.42 | 1.50 ± 0.04 |
| | Rht-A1a | 22 | 62.2 ± 0.75 | 15.3 ± 1.00 | 6.55 ± 0.39 | 16.9 ± 0.39 | 1.43 ± 0.03 |
| | Rht-A1-L358F | 17 | 67.4 ± 0.96 | 14.4 ± 0.90 | 5.88 ± 0.46 | 17.4 ± 0.45 | 1.61 ± 0.03 |
| | Rht-A1a | 16 | 67.4 ± 0.91 | 14.1 ± 1.16 | 6.13 ± 0.56 | 17.6 ± 0.48 | 1.64 ± 0.04 |
| Rht-B1a | Rht-B1b-E529K | 20 | 85.6 ± 1.19*** | 14.0 ± 0.85 | 7.75 ± 0.50 | 21.1 ± 0.49 | 1.55 ± 0.03 |
| | Rht-B1a | 27 | 95.1 ± 1.28 | 14.2 ± 0.70 | 8.30 ± 0.61 | 21.3 ± 0.40 | 1.60 ± 0.03 |
| | Rht-A1-S50F | 25 | 87.9 ± 1.10*** | 13.6 ± 0.83 | 6.63 ± 0.40 | 21.9 ± 0.37 | 1.60 ± 0.03 |
| | Rht-A1a | 23 | 94.0 ± 1.09 | 15.1 ± 0.90 | 6.52 ± 0.46 | 22.0 ± 0.34 | 1.58 ± 0.04 |
| | Rht-A1-L358F | 21 | 93.5 ± 0.66** | 18.4 ± 0.71* | 5.88 ± 0.46 | 22.2 ± 0.42 | 1.70 ± 0.03 |
| | Rht-A1a | 13 | 96.6 ± 0.93 | 15.8 ± 0.73 | 6.13 ± 0.56 | 22.0 ± 0.47 | 1.64 ± 0.03 |

^{a}n represents the number of plants in each genotypic group.
^{b}Values represent the average for each genotype ± the standard error.
*, **, and ***denote P values of <0.05, 0.01, and 0.001, respectively in individual comparisons of Rht-1 allelic groups.

TABLE 8B
The impact of Rht mutations on genotype class mean values
for seed traits from 2019 Bozeman field trials.

| Recurrent | Rht-1 | | Grain | Grain | Grain | Seeds per |
|---|---|---|---|---|---|---|
| parent | mutant | | Yield | Protein^{b} | Weight^{b} | Prod. Head^{b} |
| Rht Allele | allele | n^{a} | g/plant | g kg⁻¹ | mg/seed | No./head |
| Rht-B1b | Rht-B1b-E529K | 22 | 19.2 ± 1.69 | 173 ± 0.8 | 49.6 ± 1.14 | 47.1 ± 1.92 |
| | Rht-B1b | 16 | 16.0 ± 1.51 | 172 ± 3.8 | 47.2 ± 3.01 | 45.4 ± 2.38 |
| | Rht-A1-S50F | 23 | 18.9 ± 0.78 | 160 ± 0.9 | 44.9 ± 0.82 | 61.5 ± 2.86 |
| | Rht-A1a | 22 | 16.1 ± 1.36 | 157 ± 0.5 | 44.6 ± 0.64 | 54.4 ± 2.74 |
| | Rht-A1-L358F | 17 | 14.6 ± 1.41 | 158 ± 2.1 | 47.7 ± 0.86 | 52.8 ± 3.06 |
| | Rht-A1a | 16 | 15.7 ± 1.72 | 164 ± 4.9 | 48.8 ± 1.06 | 53.4 ± 3.06 |
| Rht-B1a | Rht-B1b-E529K | 20 | 25.6 ± 1.71 | 163 ± 3.4 | 46.4 ± 2.38 | 68.0 ± 2.01 |
| | Rht-B1a | 27 | 25.7 ± 1.47 | 166 ± 0.4 | 46.9 ± 1.80 | 63.5 ± 2.22 |
| | Rht-A1-S50F | 25 | 20.0 ± 1.52 | 176 ± 0.8 | 45.5 ± 1.95 | 63.3 ± 2.52 |
| | Rht-A1a | 23 | 17.7 ± 1.53 | 174 ± 0.5 | 46.1 ± 0.57 | 58.8 ± 2.07 |
| | Rht-A1-L358F | 21 | 24.4 ± 1.34 | 174 ± 1.8* | 47.1 ± 0.56 | 71.9 ± 0.93 |
| | Rht-A1a | 13 | 21.4 ± 1.08 | 165 ± 2.3 | 47.1 ± 0.83 | 74.9 ± 1.18 |

^{a}n represents the number of plants in each genotypic class.
^{b}Values represent the mean for each genotype ± the standard error.
*, **, and *** denote P values of <0.05, 0.01, and 0.001, respectively in individual comparisons of Rht-1 allelic groups.

### Field 2020 Trial

To expand on the 2019 field trial, ten BC1F3 lines from the mutant genotype and ten from the wild type genotype within each cross were chosen to be evaluated in short-row spaced plant trials (Tables 9a and 9b). The Rht-B1b-E529K mutation showed significant effects in the semi-dwarf background, increasing height by 16% (10.6 cm) relative to wild type, as did Rht-A1-S50F, which decreased height by 3% (1.9 cm). In the tall background, Rht-B1b-E529K was associated with an 8% (6.18 cm) decrease in height and a 4.6% (1.03 cm) decrease in flag leaf length, while the Rht-A1-S50F mutation increased flag leaf width by 4.7% (0.07 cm) and the Rht A1-L358F mutation decreased the number of spikelets per head by 3%. (0.49 spikelets per head). There was a trend amongst all the mutations in the tall background to decrease height relative to the wild type plants.

TABLE 9A
The impact of Rht mutations on genotype class mean values for agronomic
traits from 2020 field trials averaged over two Montana locations.

| Recurrent | Rht-1 | Plant | | Productive | | Flag Leaf | Flag Leaf |
|---|---|---|---|---|---|---|---|
| parent | mutant | Height^{a} | Tillers^{a} | Heads^{a} | Spikelets^{a} | Length^{a} | Width^{a} |
| Rht Allele | allele | Cm | No./plant | No./plant | No./head | cm | cm |
| Rht-B1b | Rht-B1b-E529K | 79.6 ± 1.86*** | 13.2 ± 0.45 | 10.6 ± 0.34 | 15.8 ± 0.13 | 20.9 ± 0.18 | 1.43 ± 0.01 |
| | Rht-B1b | 67.7 ± 0.52 | 13.1 ± 0.66 | 10.2 ± 0.50 | 15.8 ± 0.11 | 21.2 ± 0.47 | 1.43 ± 0.02 |
| | Genotype × | NS | NS | NS | ** | NS | * |
| | location | | | | | | |
| | Rht-A1-S50F | 56.9 ± 0.61* | 17.4 ± 0.10 | 12.7 ± 0.76 | 16.9 ± 0.48 | 19.4 ± 0.31 | 1.42 ± 0.02 |
| | Rht-A1a | 59.1 ± 0.51 | 16.3 ± 0.84 | 12.0 ± 0.66 | 16.3 ± 0.18 | 18.9 ± 0.13 | 1.39 ± 0.02 |
| | Genotype × | NS | NS | NS | NS | NS | NS |
| | location | | | | | | |
| | Rht-A1-L358F | 65.5 ± 0.54 | 15.5 ± 0.77 | 11.4 ± 0.59 | 15.6 ± 0.17 | 18.9 ± 1.18 | 1.44 ± 0.02* |
| | Rht-A1a | 65.2 ± 0.65 | 14.6 ± 0.73 | 10.9 ± 0.51 | 15.6 ± 0.14 | 19.8 ± 0.22 | 1.52 ± 0.02 |
| | Genotype × | NS | NS | NS | NS | NS | NS |
| | location | | | | | | |
| Rht-B1a | Rht-B1b-E529K | 80.0 ± 1.06** | 15.4 ± 0.96 | 11.8 ± 0.73* | 18.8 ± 0.22 | 21.9 ± 0.20** | 1.44 ± 0.02 |
| | Rht-B1a | 87.0 ± 1.57 | 14.8 ± 1.02 | 10.6 ± 0.73 | 19.4 ± 0.24 | 22.9 ± 0.27 | 1.48 ± 0.02 |
| | Genotype × | * | NS | NS | NS | NS | NS |
| | location | | | | | | |
| | Rht-A1-S50F | 81.0 ± 0.96** | 14.7 ± 0.83 | 10.8 ± 0.64 | 18.1 ± 0.18 | 22.8 ± 0.32 | 1.57 ± 0.02* |
| | Rht-A1a | 84.7 ± 1.43 | 15.0 ± 1.25 | 11.4 ± 0.92 | 18.0 ± 0.31 | 22.7 ± 0.37 | 1.48 ± 0.02 |
| | Genotype × | NS | ** | * | NS | NS | NS |
| | location | | | | | | |
| | Rht-A1-L358F | 85.9 ± 0.58 | 13.6 ± 1.01 | 10.4 ± 0.59 | 17.8 ± 0.14* | 22.6 ± 0.33 | 1.47 ± 0.03 |
| | Rht-A1a | 86.9 ± 0.47 | 14.5 ± 0.95 | 10.8 ± 0.51 | 18.3 ± 0.16 | 22.4 ± 0.34 | 1.50 ± 0.02 |
| | Genotype × | NS | NS | NS | NS | NS | ** |
| | location | | | | | | |

^{a}Values represent the average for each genotype ± the standard error.
n = 20 values in each genotype group, each value representing the average of five plants each in two replicates and in two locations.
*, **, and ***denote P values of <0.05, 0.01, and 0.001, respectively in individual comparisons of Rht-1 allelic groups and genotype × location interaction.
NS Genotype × location not significant with P value > 0.05.

TABLE 9B
The impact of Rht mutations on genotype class mean values for seed traits from 2020 field trials over two Montana locations.

| | | | | | | | | Seeds per |
|---|---|---|---|---|---|---|---|---|
| Recurrent | Rht-1 | Biomass | Straw | Grain | | Grain | Seed | Prod. |
| parent | mutant | yieldg/ | yield^{a} | Yield^{a} | Harvest | Protein^{a} | Weight^{a} | Head^{a} |
| Rht Allele | allele | plant | g/plant | g/plant | Index^{a} | g kg − 1 | mg/seed | No./head |
| Rht-B1b | Rht-B1b-E529K | 55.6 ± 1.5 | 29.4 ± 1.26 | 26.3 ± 1.21 | 0.48 ± 0.01 | 134 ± 3.1* | 45.5 ± 1.01 | 54.7 ± 1.88* |
| | Rht-B1b | 56.9 ± 2.4 | 28.9 ± 1.68 | 27.9 ± 1.67 | 0.49 ± 0.01 | 129 ± 3.1 | 43.9 ± 0.93 | 64.2 ± 4.55 |
| | Genotype × | NS | NS | NS | NS | NS | NS | |
| | location | | | | | | | |
| | Rht-A1-S50F | 50.0 ± 2.2 | 26.9 ± 1.85 | 23.0 ± 1.86 | 0.45 ± 0.01 | 137 ± 1.5 | 38.0 ± 0.94 | 47.6 ± 2.37 |
| | Rht-A1a | 47.5 ± 3.1 | 25.4 ± 1.51 | 23.0 ± 1.63 | 0.48 ± 0.01 | 135 ± 1.5 | 39.2 ± 1.02 | 48.2 ± 2.03 |
| | Genotype × | * | * | * | NS | NS | NS | |
| | location | | | | | | | |
| | Rht-A1-L358F | 45.9 ± 5.0 | 23.4 ± 2.54 | 22.6 ± 1.97 | 0.48 ± 0.01 | 128 ± 2.2 | 41.0 ± 0.88 | 52.8 ± 5.10 |
| | Rht-A1a | 47.0 ± 1.5 | 24.3 ± 1.67 | 22.6 ± 1.62 | 0.48 ± 0.01 | 127 ± 2.2 | 40.6 ± 0.86 | 48.9 ± 2.31 |
| | | NS | NS | NS | NS | NS | NS | |
| Rht-B1a | Rht-B1b-E529K | 66.7 ± 5.2 | 37.4 ± 3.19 | 29.3 ± 3.16 | 0.43 ± 0.01 | 126 ± 2.7 | 41.1 ± 1.12 | 58.3 ± 2.54 |
| | Rht-B1a | 69.5 ± 5.3 | 40.3 ± 4.06 | 29.2 ± 3.42 | 0.41 ± 0.01 | 125 ± 2.2 | 41.4 ± 1.16 | 61.9 ± 2.97 |
| | Genotype × | NS | NS | NS | NS | NS | NS | |
| | location | | | | | | | |
| | Rht-A1-S50F | 59.9 ± 3.0 | 34.7 ± 2.10 | 25.3 ± 1.62 | 0.42 ± 0.01 | 140 ± 2.9 | 40.5 ± 0.69 | 58.3 ± 2.67 |
| | Rht-A1a | 60.5 ± 2.8 | 35.7 ± 3.39 | 24.8 ± 2.44 | 0.41 ± 0.01 | 140 ± 3.2 | 40.6 ± 0.72 | 52.7 ± 1.96 |
| | Genotype × | * | * | * | NS | NS | NS | |
| | location | | | | | | | |
| | Rht-A1-L358F | 51.3 ± 2.9 | 27.4 ± 2.20 | 22.9 ± 2.07 | 0.44 ± 0.02 | 138 ± 2.8 | 38.8 ± 0.79 | 56.7 ± 3.00 |
| | Rht-A1a | 53.3 ± 2.5 | 30.1 ± 2.6 | 23.3 ± 2.22 | 0.42 ± 0.02 | 139 ± 2.2 | 38.1 ± 0.93 | 53.5 ± 1.77 |
| | Genotype × | NS | NS | NS | NS | NS | NS | |
| | location | | | | | | | |

^{a}Values represent the mean for each genotype ± the standard error.
n = 20 values in each genotype group, each value representing the average of 5 plants each in two replicates and in two location.
*and ** Denotes a P value of <0.05 and <0.01 in individual comparisons of Rht-1 allelic groups and genotype × location interaction.
NS Genotype × location not significant with P value > 0.05.

### GA Responsiveness Assay: Coleoptile Length

The impact of durum Rht mutations on coleoptile length was evaluated in the presence and absence of GA (Table 10). Length was compared between the mutant and wild type in each cross, and between the GA and water treatments. This allowed us to determine both the effects of the mutations on coleoptile length and on the impact of GA on coleoptile length. The Rht-B1b-E529K mutation significantly affected coleoptile length in both the semi-dwarf and full height backgrounds, reducing coleoptile length by 20% (18.44 cm) (P<0.001) without GA present and 28% (29.23 cm) (P<0.001) with GA in the full height background. Coleoptile length in the semi-dwarf background was increased by 23% (16.71 mm) (P<0.01) in water and 25% (20.12 mm) (P<0.01) in the GA solution. There were no significant differences between the sister lines containing Rht-A mutations. When comparing the length of the coleoptile in GA to that in water for the same genotype, the presence of GA increased coleoptile length by 11% for the wild-type line in the Rht-B1b-E529K mutation in both backgrounds and increased length by 8% in the wild type line in the full-height background Rht-A1-L358F pair.

TABLE 10
The impact of Rht mutations on coleoptile length and GA responsiveness.

| Recurrent | | | Coleoptile | Coleoptile | |
|---|---|---|---|---|---|
| parent | Rht-1 mutant | | length in | length in GA | GA/ |
| Rht allele | allele | n^{a} | water (mm)^{b} | (mm)^{b} | Water^{c} |
| Rht-B1b | Rht-B1b-E529K | 10 | 88.0 ± 4.24** | 99.4 ± 4.60** | 1.13 |
| | Rht-B1b | 10 | 71.3 ± 1.55 | 79.3 ± 2.54 | 1.11** |
| | Rht-A1-S50F | 10 | 64.7 ± 2.31 | 65.4 ± 2.50 | 1.01 |
| | Rht-A1a | 10 | 63.1 ± 1.50 | 60.4 ± 1.90 | 0.96 |
| | Rht-A1-L358F | 10 | 62.4 ± 1.60 | 58.9 ± 1.38 | 0.94 |
| | Rht-A1a | 10 | 60.5 ± 2.01 | 58.3 ± 1.62 | 0.96 |
| Rht-B1a | Rht-B1b-E529K | 10 | 74.6 ± 2.43*** | 74.9 ± 2.73*** | 1.00 |
| | Rht-A1a | 10 | 93.1 ± 3.78 | 104.1 ± 2.26 | 1.11* |
| | Rht-A1-S50F | 10 | 107.7 ± 2.20 | 114.3 ± 2.84 | 1.06 |
| | Rht-A1a | 10 | 107.9 ± 2.40 | 113.4 ± 3.19 | 1.05 |
| | Rht-A1-L358F | 10 | 104.9 ± 2.34 | 109.5 ± 2.38 | 1.04 |
| | Rht-A1a | 10 | 106.7 ± 2.64 | 115.7 ± 2.17 | 1.08* |

^{a}n represents the number of values in each genotype after averaging two replicates together
^{b}Values represent the average for each genotype ± the standard error
^{c}Difference between GA and water treatments expressed as a ratio.
*, **, and *** denote P values of <0.05, 0.01, and 0.001 respectively.

### GA Responsiveness Assay: GID1 Interaction

Durum Rht mutations and their interaction with the GID1 receptor was tested in vitro with a yeast-2-hybrid assay (FIG. 7). The positive controls Rht-A1a and Rht-B1a showed strong interaction on both media types, while the GID1/empty vector negative control showed no interaction. Rht-B1b-E529K also showed no interaction on either media, indicating that the addition of the E529K mutation to Rht-B1b does not alter its effect in vitro. The missense mutations had a varied effect. Rht-A1-S50F showed no interaction without GA, but restored interaction near the level of wild type when GA was present. Rht-A1-L358F demonstrated a weaker interaction than wild type in the absence of GA, and a strong interaction similar to wild type when GA was present.

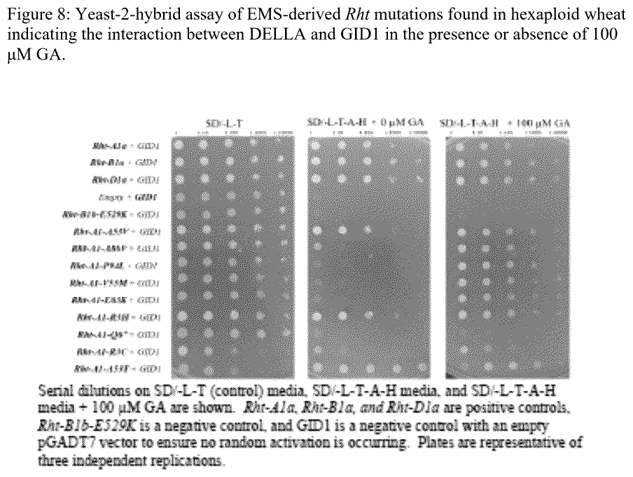

The same interaction assay was performed on a series of Rht-A1 EMS mutations identified in hexaploid wheat (FIG. 8). Positive controls Rht-A1a, Rht-B1a, and Rht-D1a showed strong interaction with GID1 in both the presence and absence of GA, while negative controls Rht-B1b-E529K and GID1/empty vector did not. Rht-A mutations varied in their effect. Rht-A1-A53V, Rht-A1-R3H, and Rht-A1-A53T each showed interaction close to that of the positive controls when no GA is present, while Rht-A1-A86V and Rht-A1-R3C showed interaction only at the strongest concentration. Rht-A1-P94L, Rht-A1-V55M, and Rht-A1-E63K did not grow on the no-GA plate. The addition of GA restored the interaction of Rht-A1-A53V, Rht-A1-A86V, Rht-A1-P94L, Rht-A1-V55M, Rht-A1-E63K, Rht-A1-R3H, and Rht-A1-A53T to the same level as the wild-type positive controls. Rht-A1-Q6* mimicked the negative controls, with no growth on either plate. Both Rht-A1-A53T and Rht-A1-R3C had reduced growth on the control plate, but in comparing the interaction plates still showed enough growth to interpret.

### Discussion

Two Rht-A missense mutations and the previously reported Rht-B1b-E529K allele were tested to see impacts on various plant physical traits and grain characteristics. Sister lines were created using each of the mutations in two different Montana-adapted parent lines, for a total of 6 mutant genotypes to be compared to their corresponding wild type sister line (Table 7).

In both the BC₁F₂ (Table 8a) and BC₁F₃ (Table 9a) generations, we observed the height increasing effect of Rht-B1b-E529K when crossed to a semi-dwarf parent, as previously reported (Mo et al. 2018). The effect was greater in 2019, with a 22% increase versus a 16% increase in 2020. We also found that the Rht-B1b-E529K decreased plant height when crossed to a full height parent. Again, the effect was more pronounced in the 2019 trial with an 11% reduction compared to an 8% reduction in 2020. Both the semi-dwarf and the full-height crosses to Rht-B1b-E529K resulted in an intermediate-height durum plant, between current semi-dwarf and full height varieties.

When comparing the Rht-A mutations to their semi-dwarf sister lines, neither Rht-A1-S50T nor Rht-A1-L358F provided the desired height increase. Both Rht-A missense mutations showed significant height reduction compared to their wild type sister lines in 2019 but that trend was not statistically significant in 2020. One possible reason for the differing results between years may be that plants overall were taller in 2019 than 2020, potentially due to a wetter year. Another possibility is that the plots were located in slightly different locations within the farm each year and may have had underlying soil differences that influenced plant growth. None of the mutations tested here contributed to an increased yield (Tables 8b and 9b).

Although no height-reducing Rht-A alleles have been identified in hexaploid wheat (Flintham et al. 1997, Gale and Youssefian 1985, Pearce et al. 2011), this data suggests that when integrated into durum varieties with the wild type Rht-B1a allele, Rht-A missense mutations could bring about shorter plants. Rht-B1b-E529K was shown to be useful for creating intermediate height durum plants both when combined with Rht-B1b, as shown in (Mo et al. 2018), and with Rht-B1a.

Along with field trials to test the agronomic impacts of the mutations, two GA responsiveness assays were also conducted. The interaction between GID1, a known receptor of wild type Rht when GA is present, and various Rht-A1 mutations was analyzed in-vitro with yeast-2-hybrid assays. Pearce et al. (2012) showed the Rht-A1a, Rht-B1b, and Rht-D1b all have DELLA/GID1 interactions, while there is no interaction with Rht-B1b, either in the presence or absence of GA. In this experiment, the co-transformed yeast was spotted onto three types of media to view the interactions in the presence of GA, in the absence of GA, and on a nonselective media as a control. All three mutations in the durum field trials were included (FIG. 7), as well as a second assay that examined 9 Rht-A mutations found in hexaploid wheat (FIG. 8). We observed no growth on either plate for Rht-B1b-E529K when examining the durum mutations, and since Pearce et al. 2012 observed that Rht-B1b did not grow, it is shown that the in-vitro effect of Rht-B1b is not altered by the addition of the E529K mutation. Therefore, it was included in the hexaploid mutation assay as a negative control.

Of the mutations tested, only one was a nonsense mutation. Rht-A1-Q6* did not grow on either plate, showing the same pattern of no interaction when a stop mutation is present that was demonstrated by Pearce et al. (2012) and with the Rht-B1b-E529K mutation in this study, but disagrees with Jobson et al. (2020, in press) who found that GA restored the function of three stop mutations.

The missense mutations varied in effect. Rht-A1-S50F, Rht-A1-P94L, Rht-A1-V55M, and Rht-A1-E63K had no interaction without GA, but their function was fully restored when GA was present. Rht-A1-L358F and Rht-A1-A86V interacted slightly on the no-GA plates, while Rht-A1-A53V and Rht-A1-R3C showed intermediate interaction, midway between the positive and negative controls. With each of those four, the addition of GA restored full function. Rht-A1-A53T's growth appeared similar on both plates but reduced on the control plate which leads to a less reliable interpretation of its results. Alleles that grew on both plates similar to wild type alleles are expected to exhibit plant growth similar to wild type plants, whereas alleles that differed when GA was or was not present are expected to produce plants that are intermediate in height.

Rht-B1b and Rht-D1b reduce coleoptile length compared to wild type Rht alleles (Fick and Qualset 1979, Schillinger et al. 1998, Jobson et al. in press 2020, Amram et al. 2015, Liatukas and Ruzgas 2011), and the Rht-B1b-E529K mutation partially repressed that length reduction compared to Rht-B1b (Mo et al. 2018). We observed a coleoptile length increase compared to Rht-B1b, which agrees with the results of the durum experiment described above, and a length decrease compared to Rht-B1a (Table 10). These results mirror the effect seen in field-grown plants of the same genotypes, as observed in previous studies correlating height of plants to coleoptile length (Fick and Qualset 1979, Schillinger et al. 1998, Liatukas and Ruzgas 2011, Jobson et al. in press 2020). The Rht-B1b-E529K mutation's effect on coleoptile length was increased when GA was present, 28% versus 20% reduction in the Rht-B1a cross and 25% versus 23% increase in the Rht-B1b cross. This mutation-induced increased GA-sensitivity response was not observed in the other crosses examined, nor was a difference in coleoptile length detected between sister lines, which supports Jobson et al. (in press 2020)'s findings that mutations other than Rht-B1b and Rht-D1b did not impact coleoptile growth or GA responsiveness. The wild type sister line in the Rht-A1-L358F, Rht-B1a cross did show a GA response, with an increased coleoptile length of 8% when GA was present. This supports previous studies (Fick and Qualset 1979, Schillinger et al. 1998, Liatukas and Ruzgas 2011, Jobson et al. in press 2020) showing that wild type Rht-B1a plants are GA-responsive and grow longer coleoptiles when exposed to GA. Field experiment results showed that height was unaffected by Rht-A mutations when Rht-B1b was present, but when combined with Rht-B1a the mutations did provide a height decrease. Coleoptile data revealed a similar pattern. The yeast-2-hybrid results indicated that both Rht-A mutations would confer intermediate interaction with GID1, which may produce plants intermediate in height. These results indicate that by adding Rht-A mutations to full height durum varieties, plant height can be reduced, while the same technique in semi-dwarf varieties is not likely to show the desired intermediate height plants.

### Example 4: Second TB1 Research Study

Overexpression of TB1 is associated with decreased tillering (Xu et al. 2014, Sakamoto and Matsuoka 2004), while wild-type TB1 (and orthologs) plants exhibit apical dominance with few main branches (Aguilar-Martinez et al. 2007, Doebley et al. 1995, Doebley et al. 1997, and Takeda et al. 2003) and mutant TB1 plants reveal a bushier architecture consisting of more tillers (Cubas et al. 1999, Xu et al. 2014, Kebrom et al. 2006, and Liu et al. 2018) and increased height (Dixon et al. 2020). We hypothesized that tiller number can be increased and utilized as a method to increase total biomass and/or grain yield in durum and other wheat relative species by suppressing TB1 expression by selecting for TB1 null alleles.

To analyze the effects of TB1 null mutations in durum, an EMS mutagenized durum wheat population was screened for stop codon mutations. Three lines containing TB1 stop mutations were chosen for crossing into wild type durum, then greenhouse and field trials were conducted on the populations created from those crosses to evaluate phenotypic differences including plant and root biomass, height, and tillering. Additionally, an expression study using RNAseq was conducted to determine the effect the mutations had on the expression of genes involved in seed dormancy and germination in developing meristems. A screen of commonly grown Montana winter, spring, and durum wheat was also conducted to determine whether commonly grown wheat varieties vary in TB1 alleles. A study on root biomass was conducted to determine if above-ground phenotypic differences were reflected below ground as well.

### Materials and Methods

### Plant Material

The wheat TILLING population database (UC Davis, Tsai et al. 2011, Krasileva et al. 2017) was screened using online tools for TB1 null mutations in the Triticum turgidum variety “Kronos” (D03-21) and three lines containing TB1 stop mutations were chosen for this project: TB1-A1-W339*, TB1-B1-W341*, and TB1-B1-Q142* (see Table 11). M4 seed of those lines was obtained and M4 plants grown in fall 2018. F1 crosses between the TB1-A1-W339* line and both TB1-B1 mutant lines were made and the resulting F1 seeds were planted, grown, and allowed to self-pollinate. DNA was extracted from the F2 plants, then PCR was performed using primers shown in Table 12. Conditions for PCR were 40 cycles of 30s denaturation at 96° C., 30 s annealing at 65° C., and 60 s extension at 72° C., for both primer sets. PCR products were Sanger sequenced (GENEWIZ, Inc., Cambridge, MA, USA), then analyzed using the DNASTAR SeqMan Pro software (DNASTAR Version 15.0.1.1, Madison, WI, USA) against the reference genes from Liu et al. (2017). Only homozygous mutant and homozygous wild type plants were kept for advancement. In this manner, four genotypes of interest for each cross were created: AABB, aabb, AAbb, and aaBB for both TB1-A1-W339* x TB1-B1-W341* and TB1-A1-W339* x TB1-B1-Q142*. F2 homozygotes were grown and allowed to self-pollinate to produce seed of F2-derived F3 lines for planting in the field during spring 2019. The number of F2-derived F3 lines varied from 9 to 19 in each of the four genotype classes for each cross.

Table 11: Summary of TS1 mutations.

| | DNA change (base | Original | New |
|---|---|---|---|
| Allele | pairs from start codon) | Codon | Codon |
| TB1-A-W339Stop | 1016 | TGG | TAG |
| TB1-B-W341Stop | 1023 | TGG | TGA |
| 7B1-B-Q1425tog | 424 | CAG | TAG |

Table 12: Amplification and sequencing
primers used to detect TB1 mutations.

| | | | PCR |
|---|---|---|---|
| Mutation | | | Product |
| Detected | Primer Sequence^{a} | Primer Type | Size |
| TB1-A1-W339* | 5′ GATCGACAACCAGCCGCCA 3′ | Forward | 882 bp |
| | 3′ GGGCTGGGAGTTGGGAAA 5′ | Reverse | |
| | 5′ GACAAGGAGTCGAGGACG 3′ | Sequencing | |
| TB1-B1-W341* | 5′ GATCGACAACCAGTGGCCG 3′ | Forward | 859 bp |
| | 3′ GGGCTGGGAGTTGGGAAA 5′ | Reverse | |
| | 5′ GACAAGGAGTGGAGGACG 3′ | Sequencing | |
| TB1-B1-Q142* | 5′ GATCGACAACCAGTCGCCG 3′ | Forward/ | 859 bp |
| | | Sequencing | |
| | 3′ GGGCTGGGAGTTGGGAAA 5′ | Reverse | |

^{a}Primers developed from sequence alignment from Liu et al. 2018. (Genbank accession numbers TraesCS4A02G271300 and TraesCS4B02G042700).

### Greenhouse Growing Conditions

Seeds were planted in Sunshine mix (Sungro Horticulture, Agawam, MA, USA) potting soil, 4 seeds per 20.3 cm pot. Plants were watered daily with a 100 ppm solution of 20-20-20 fertilizer applied through a Dosatron (Dosatron International, Inc., Clearwater, FL, USA) once plants reached the three-leaf stage. The temperature was set at 22.2° C. in the daytime and 18.3° C. at night. After flowering, the temperature was increased to 25° C. day and 21° C. until harvest. A micronized sulfur solution (Bonide Products, Inc., Oriskany, NY, USA) at a rate of 4.38 g/l was applied to plants at the 2-3 leaf stage (Z 12-Z13 on the Zadoks growth scale for wheat) for powdery mildew and mite prevention, and Marathon (Imidacloprid 1%, OHP, Inc., Bluffton, SC, USA) at a rate of 2.7 g per pot was applied at the same growth stage for aphid control.

### Initial Greenhouse Trial

A single randomized replicate of the F2 lines (102 entries) were grown in the greenhouse under the above-described conditions to produce seed for field trials. These plants were also evaluated for genotypic differences to confirm that field trials were warranted.

### Field Experiment

The F2-derived F3 lines (102 entries) were grown in two replications of a randomized complete block design at the Arthur H. Post Agronomy Farm near Bozeman, MT (latitude 45.67N, longitude 111.15W, elevation 1,455 m, soil type is Amsterdam silt loam). The plants were grown in 3-m rows with 30 cm spacing between each row. Within a row, seeds were sown 15 cm apart with 13 plants per row. Seeds were hand planted to a depth of 2/5 cm on May 9, 2019. After testing soil, 185 kg/ha urea (46-0-0) was added. From May 1st to August 31st, the research station received 18.62 cm of precipitation. The highest recorded air temperature was on July 23 at 32.3° C., the lowest recorded air temperature was −4.18° C. on May 1. Five cm irrigation water was applied using hand line sprinklers on July 1. A 1.1 liter/hectare application of Huskie (Bayer CropScience, Research Triangle PK, NC, USA) was applied for broadleaf weed control, and throughout the growing season weeds were rogued out by hand. F2-derived F4 seed harvested from the 2019 trial was used for similar field trials in 2020 at the Arthur H. Post Agronomy Farm and at the Central Agricultural Research Center (CARC) near Moccasin, MT (latitude 47.06 N, longitude 109.96W, elevation 1,297 m, soil type Danvers-Judith clay loam). Experimental design at both locations was a randomized complete block design with two replications with 93 entries per replication. Plots were 1.5-m rows with 30-cm spacing between rows. Within a row, seeds were sown 15 cm apart with eight plants per row.

Plots were hand-planted to a depth of 2.5 cm on Apr. 25, 2020 at the Bozeman location and on May 11, 2020 at the Moccasin location. The Moccasin plot was fertilized with 67.3 kg/ha ESN slow-release nitrogen fertilizer (44-0-0, Nutrien, Saskatoon, SK, CA) and in Bozeman with 403.5 kg/ha urea (46-0-0). The Bozeman trial received 5 cm irrigation water on July 2, while the Moccasin location received no added irrigation. From May 1 to August 31, the Post farm received 14.3 cm of precipitation. The highest recorded air temperatures were on August 17 and 18 at 34.4° C., the lowest recorded air temperature was −0.6° C. on May 8 and 11. The CARC received 16.03 cm of precipitation. The highest recorded air temperature was on August 17 at 33.9° C., the lowest recorded air temperature was −1.1° C. on May 12. Herbicides applied to the Bozeman crop were 0.06 liter/hectare of Affinity Broadspec (FMC, Philadelphia, PA, USA), 0.58 liter/hectare MCPA Ester 4 (Albaugh, LLC, Ankeny, IA, USA), 1.2 liter/hectare Discover NG (Syngenta Crop Protection, LLC, Greensboro, NC, USA), and 0.29 liter/hectare Propi-Star EC (Albaugh, LLC, Ankeny, IA, USA) on June 1. In Moccasin, Vendetta (Wilbur-Ellis Agribusiness, Aurora, CO, USA) was applied on May 27 for broadleaf weed control.

### Plant Measurements

Greenhouse and field measurements were conducted similarly. Plants were measured near physiological maturity, after growth had ceased. Height of plant was determined by measuring from the soil surface to the terminal spikelet of the tallest tiller. Tiller number was counted at the base of the plant, regardless of whether the tiller produced a head. Spikelets per head was counted on the tallest tiller only. Flag leaf length was measured from stem to tip, and width at the widest point of the leaf. Number of productive heads included only heads that formed seed. Biomass was measured at harvest by weighing the whole plant after cutting at the base and adjusted for moisture content and seed weight after drying and harvesting seed. Seed protein and moisture were measured using a Foss Infratec 1241 Machine (Foss Analytics, Eden Prairie, MN, USA; AACCI Method 39-11.01). Individual seed weight was measured with a Single Kernel Characterization System 4100 (Perten, Springfield, IL, USA; AACCI Method 55-31.01).

Field data was measured on five consecutive plants in the middle of each row, then averaged for one data point per row. The five plants were tagged prior to measurements to ensure consistency, then harvested together. Greenhouse plants were measured individually, for one data point per plant.

### RNA Sequencing Expression Analysis

F4 seed of each genotype (AABB, aabb, AAbb, and aaBB) from the Tb1-A1-W339*/Tb1-B1a, Tb1-A1a/Tb1-B1-Q142* cross population was planted in the greenhouse in 20.3 cm pots, with 15 seeds per pot. Three different lines from each genotype were planted in individual pots for three biological replicates, for a total of 45 seeds per genotype, repeated three times for three technical replicates. Developing spikes (Dixon et al., 2018) at 3 weeks post planting were used for analyses. Plants were removed from soil and rinsed, then ˜1 cm of tiller tissue was excised. A composite sample of 45 mg tissue was collected and immediately frozen in liquid N. RNA extraction was completed with a Qiagen RNeasy Plant Mini Kit and the protocol listed therein (Qiagen Catalog #74904, 4th edition, April 2006). RNA samples were analyzed using GENEWIZ Next Generation Sequencing service (GENEWIZ, South Plainfield, NJ, USA).

### Variety Screen

Fifteen winter wheat varieties, 15 spring wheat varieties, and 10 durum varieties along with controls of Chinese spring wheat and one representative line of each of the four genotypes of interest (Kronos AABB, aabb, AAbb, and BBaa) were planted in the greenhouse (Table 13), then DNA extracted at the two-leaf stage. Each line was genotyped with primers designed to capture the whole TB1 gene (see Table 14 for primer sequences and product size). PCR conditions were as follows: initialization 96° C. for 5 min., then 40 cycles of 96° C. for 30 sec, 30 sec at the annealing temperature shown in Table 14, 1 minute at 72° C., and a 7 min. final extension at 72° C. PCR products were Sanger sequenced by GENEWIZ (GENEWIZ, Inc., Cambridge, MA, USA), then analyzed for differing alleles using the DNASTAR SeqMan Pro software (DNASTAR Version 15.0.1.1, Madison, WI, USA) against the reference genes from Liu et al. (2017).

TABLE 13
List of wheat varieties screened for TB1 alleles.

| Variety/Name | Type | Identifier |
|---|---|---|
| Alum | Spring | WA8166 |
| Brennan | Spring | Syngenta 2009 |
| Chinese Spring | Spring | CI 14108 |
| Choteau | Spring | PI 633974 |
| Corbin | Spring | WestBred, LLC 2006 |
| Duclair | Spring | PI 680981 |
| Egan | Spring | PI 671855 |
| Lanning | Spring | PI 676978 |
| McNeal | Spring | PI 574642 |
| NS Presser CL | Spring | Northern Seed LLC 2016 |
| Reeder | Spring | PI 613586 |
| SY Ingmar | Spring | Syngenta 2015 |
| SY Soren | Spring | Syngenta 2011 |
| SY Valda | Spring | Syngenta 2015 |
| Vida | Spring | PI 642366 |
| WB Gunnison | Spring | WestBred, LLC 2011 |
| Bearpaw | Winter | PI 665228 |
| Bobcat | Winter | PI 683235 |
| Decade | Winter | PI 660291 |
| Flathead | Winter | PI 693237 |
| FourOSix | Winter | PI 689753 |
| Judee | Winter | PI 665227 |
| Loma | Winter | PI 680576 |
| MT1435 | Winter | MSU experimental line |
| Northern | Winter | PI 676026 |
| Rampart | Winter | PI 593889 |
| Ray | Winter | PI 689754 |
| StandClear CLP | Winter | PI 693236 |
| SY Clearstone 2CL | Winter | Syngenta 2012 |
| Warhorse | Winter | PI 670157 |
| Yellowstone | Winter | PI 643428 |
| Alzada | Durum | WestBred, LLC 2004 |
| CDC-Precision | Durum | CFIA 7832, 2015 |
| CDC-Vivid | Durum | CFIA #7220, 2012 |
| Divide | Durum | PI 64021 |
| Joppa | Durum | PI 673106 |
| Mountrail | Durum | PI 607540 |
| MT112219 | Durum | MSU experimental line |
| MTD16005 | Durum | MSU experimental line |
| ND-Riveland | Durum | PI 687796 |
| Tioga | Durum | PI 660664 |

TABLE 14
Primers used for TB/ mutation screen.

| | | Product | Annealing |
|---|---|---|---|
| | | Size | Temp. |
| Name* | Primer Sequence | bp | ° C. |
| Tb-A-FLW | 5′ CGAGCCCCATGGACTTA 3′ | 1064 | 58 |
| TB1-ABD-ALW | 3′ CTTGTACGGTACGCATGTC 5′ | | |
| Tb-8-FLW | 5′ CATTCCTCCCCTGCATTT 3′ | 981 | 58 |
| TB1-ABD-RLW | 3′ CTTGTACCGTACGCATGTC 5′ | | |
| Th-D-FLW | 5′ GGTGCAATGGCTCCTCG 3′ | 634 | 59 |
| TB1-ABD-RLW | 3′ CTTGTACCGTACATGTC 5′ | | |
| TB1-A Frag 2 For | 5′ GCACATCTGATCATAGCTCCT 3′ | 1191 | 66 |
| TB1-A Frag 2 Rev | 3′ GGGCTGCGAGTTGGGAAA 5′ | | |
| TB1-8 Frag 2 For | 5′ CATGCCCTTTCCACCAC 3′ | 1033 | 61 |
| Tb-B1-RevLW | 3′ GAGGAGCCATTGCACCGTC 5′ | | |
| TB1-D Frag 2 For | 5′ AGACGGGTGGTCTCAGGGT 3′ | 1094 | 65 |
| TB1-D Frag 2 Rev | 3′ GGGCTGCGAGTTGGTAAA 5′ | | |

Gene IDs used for developing primers for TB1-A1, TB1-B1, and TB1-D1 are TresCS4A02G271300, TraesCS4B02G042700, and TraesCS4D02G040100, respectively.

### Root Analysis

F4 seed of each genotype (AABB, aabb, AAbb, and aaBB) from the Tb1-A1-W339*/Tb1-B1a, Tb1-A1a/Tb1-B1-Q142* cross population was planted in the greenhouse in 20.3 cm pots filled with ceramic granules (PROFILE Products LLC, Buffalo Grove, IL, USA), with eight seeds per pot, thinned to four spaced plants per pot when plants were at the two-three leaf stage. Three different lines from each genotype were planted in individual pots for three biological replicates, repeated three times for three technical replicates. This experiment was duplicated three times, for two data collection time points: one set one month after planting and two sets two months after planting. Pots were placed randomly into blocks by time point. Plants were gently removed from the ceramic granules, then rinsed in water to remove any granules clinging to the roots. Total roots in each pot were compiled in a paper bag for drying, with the same being done with the above-ground biomass. Tiller counts were also taken.

### Statistical Analysis

Root data was analyzed using a one-way ANOVA treating each date separately. Pairwise comparisons among genotype means were conducted using the emmeans package (Lenth 2020) for R data analysis software (R Foundation for Statistical Computing, Vienna, Austria). The mean for each line over both replicates in each field trial environment was computed, then data was combined over all three environments before a mixed linear model was fitted in R using the Imer4 package in R (Bates et al., 2015) where the model included location, genotype class, lines within genotype class, and location x genotype class interaction. Lines within genotype class were considered random and other factors were fixed. When genotype x environment interaction was present, each location was analyzed separately. Dunnett's test for comparing groups to a control was performed using the DescTools package in R (Signorell et al. 2020). Greenhouse data were also analyzed with one-way ANOVA, and Tukey's test for determining differences amongst all groups was performed using the agricolae package (de Mendiburu 2020) for R (R Foundation for Statistical Computing, Vienna, Austria). RNA data was first normalized to Actin using the method in (Oistad et al. 2016), then analyzed with one tailed t-tests to detect differences in fold change for expression of genes in mutant genotypes versus the TB1 wild type sample.

### Results

### Initial Greenhouse Trial

To test the impact of TB1 mutations on plant growth under controlled conditions, F2 TB1 homozygous plants were grown in the greenhouse. Tillers and spikelets were counted at plant maturity. Total tillers, productive tillers, spikelets per head, and productive spikelets per plant were all compared across genotypes for each cross (Tables 15a and 15b). There were no statistically significant differences at the P<0.05 level among genotypes for either cross, however, the trend for both was towards increased total and productive tillers when one or more TB1 null mutations were present.

TABLE 15a
Tiller, spikelet, and productive head comparisons of genotype class
means from the [symbol] cross population in greenhouse trial.

| | | | Productive | |
|---|---|---|---|---|
| | | Total Tillers | Tillers | Spikelets/ |
| | | (mean ± SE, | (mean ± SE, | Head |
| Genotype | n | No./plant) | No./plant) | (mean ± SE) |
| [symbol] | 15 | 6.27 ± 0.54 [symbol] | 2.93 ± 0.21 [symbol] | 14.49 ± 0.29 [symbol] |
| [symbol] | 18 | 7.00 ± 0.49 [symbol] | 2.72 ± 0.19 [symbol] | 13.66 ± 0.26 [symbol] |
| [symbol] | 7 | 6.71 ± 0.79 [symbol] | 2.57 ± 0.31 [symbol] | 13.18 ± 0.42 [symbol] |
| [symbol] | 9 | 7.89 ± 0.69 [symbol] | 3.00 ± 0.27 [symbol] | 14.12 ± 0.37 [symbol] |

^{a}Numbers with different superscript letters differ at P < 0.05. [symbol]
indicates data missing or illegible when filed

TABLE 15b
Tiller, spikelet, and productive head comparisons of genotype class
means from the [symbol] cross population in greenhouse trial

| | | | Productive | |
|---|---|---|---|---|
| | | Total Tillers | Tillers | Spikelets/ |
| | | (mean ± SE, | (mean ± SE, | Head |
| Genotype | n | No./plant) | No./plant) | (mean ± SE) |
| [symbol] | 13 | 5.38 ± 0.39 [symbol] | 2.92 ± 0.19 [symbol] | 13.83 ± 0.28 [symbol] |
| [symbol] | 12 | 6.42 ± 0.41 [symbol] | 2.83 ± 0.20 [symbol] | 13.19 ± 0.29 [symbol] |
| [symbol] | 13 | 6.54 ± 0.39 [symbol] | 2.92 ± 0.19 [symbol] | 13.27 ± 0.28 [symbol] |
| [symbol] | 12 | 6.17 ± 0.41 [symbol] | 3.42 ± 0.20 [symbol] | 13.07 ± 0.29 [symbol] |

^{a}Numbers with different superscript letters differ at P < 0.05. [symbol]
indicates data missing or illegible when filed

### Field Trials

To test the impact of TB1 null mutations on plant growth under field conditions, F2: F3 homozygous plants were grown in 2019 at one location and F2: F4 lines 10 derived from that trial were grown in 2020 at two locations. Only tiller number in the Tb1-A1-W339*/Tb1-B1a, Tb1-A1a/Tb1-B1-Q142* cross differed significantly by environment. The double mutant genotype had 27% more tillers than the wild type, and 18-21% more than the mutant groups in 2019, but no differences were found in either of the 2020 locations (Table 16).

TABLE 16
Tiller number comparisons of genotype class means from the [symbol]
cross population grown in three environments.

| | | Bozeman | Bozeman | Moccasin |
|---|---|---|---|---|
| | | 2019 | 2020 | 2020 |
| Genotype | n | Tillers/plant | Tillers/plant | Tillers/plant |
| [symbol] | 11 | 16.2 ± 0.58 [symbol] | 15.9 ± 0.71 [symbol] | 11.3 ± 0.67 [symbol] |
| [symbol] | 10 | 17.6 ± 0.56 [symbol] | 16.4 ± 0.53 [symbol] | 11.4 ± 0.50 [symbol] |
| [symbol] | 11 | 17.3 ± 0.90 [symbol] | 15.5 ± 0.52 [symbol] | 12.4 ± 0.64 [symbol] |
| [symbol] | 10 | 21.0 ± 0.80 [symbol] | 16.5 ± 0.85 [symbol] | 12.5 ± 0.46 [symbol] |

^{a}Numbers with different superscript letters differ at P < 0.05.
n denotes the number of ID's in each genotype averaged across two reps, in each environment. [symbol]
indicates data missing or illegible when filed

Finding no other GxE interactions, analysis was continued by averaging the two replications in an environment together, then combining the three environments. As shown in Tables 17a and 17b, the double mutant TB1-A1-W339*, TB1-B1-Q142* had reduced spikelets and reduced flag leaf length, while TB1-A1-W339*, TB1-B1 reduced only flag leaf length. Number of tillers when mutations were present trended upwards, as did productive heads, biomass and plant height, and individual seed weight trended down.

[symbol]

| | | |
|---|---|---|
| [symbol] | [symbol] | [symbol] |
| [symbol] | [symbol] | [symbol] |
| [symbol] | [symbol] | [symbol] |
| [symbol] | [symbol] | [symbol] |

[symbol]

| | | |
|---|---|---|
| [symbol] | [symbol] | [symbol] |
| [symbol] | [symbol] | [symbol] |
| [symbol] | [symbol] | [symbol] |
| [symbol] | [symbol] | [symbol] |

[symbol]

indicates data missing or illegible when filed

TABLE 17A
Comparison of agronomic trait measurements taken on each of the four
genotypes produced in the Tb1-A1-W339*/Tb1-B1a, Tb1-A1a/Tb1-B1-Q142*
cross, from field trials combined across three Montana environments.

| | | Plant | | | Productive | Flag Leaf | Flag Leaf |
|---|---|---|---|---|---|---|---|
| | | Height | Tillers | Spikelets | Heads | Length | Width |
| TB1 Genotype | N^{a} | cm | No./plant | No./head | No./plant | cm | cm |
| TB1-A1, TB1-B1 | 11 | 62.2 ± 0.98 | 14.5 ± 0.54b | 14.9 ± 0.12 | 11.4 ± 0.54b | 18.3 ± 0.25a | 1.45 ± 0.02 |
| TB1-A1, TB1- | 10 | 62.5 ± 0.82 | 15.2 ± 0.58b | 14.6 ± 0.13 | 12.2 ± 0.58b | 17.9 ± 0.25a | 1.43 ± 0.02 |
| B1- Q142* | | | | | | | |
| TB1-A1-W339*, | 11 | 64.7 ± 0.93 | 15.1 ± 0.53b | 14.8 ± 0.10 | 12.2 ± 0.55b | 17.3 ± 0.24b | 1.44 ± 0.01 |
| TB1-B1 | | | | | | | |
| TB1-A1-W339*, | 10 | 65.1 ± 1.01 | 16.7 ± 0.76a | 14.4 ± 0.15 | 13.4 ± 0.71a | 16.9 ± 0.25b | 1.41 ± 0.02 |
| TB1-B1-Q142* | | | | | | | |
| Prob > F | | 0.22 | 0.026 | 0.13 | 0.017 | <0.001 | 0.68 |

^{a}Denotes the number of lines in each genotype class.
Values represent the average for each genotype ± the standard error.
Means followed by different letters indicate they differ significantly (P < 0.05). T comparisons were made only following a significant (P < 0.05) F ratio.

TABLE 17B
Comparison of seed trait measurements taken on each of the four genotypes
produced in the Tb1-A1-W339*/Tb1-B1a, Tb1-A1a/Tb1-B1-Q142* cross,
from field trials combined across three Montana environments.

| | | Straw | Grain | | Seed | | |
|---|---|---|---|---|---|---|---|
| | | yield | yield | Protein | Weight | Biomass | Harvest |
| TB1 Genotype | N^{a} | g/plant | g/plant | % | mg/seed | g/plant | Index |
| TB1-A1, TB1-B1 | 11 | 20.4 ± 1.11 | 19.9 ± 1.39 | 14.3 ± 0.11 | 42.5 ± 0.88 | 40.4 | 0.48 ± 0.02 |
| TB1-A1, TB1- | 10 | 21.3 ± 0.96 | 20.7 ± 1.25 | 14.4 ± 0.16 | 44.3 ± 0.77 | 41.9 | 0.49 ± 0.02 |
| B1-Q142* | | | | | | | |
| TB1-A1-W339*, | 11 | 20.8 ± 0.95 | 20.5 ± 1.30 | 14.4 ± 0.18 | 42.7 ± 0.68 | 41.4 | 0.49 ± 0.02 |
| TB1-B1 | | | | | | | |
| TB1-A1-W339*, | 10 | 22.6 ± 1.45 | 20.5 ± 1.29 | 14.2 ± 0.28 | 41.9 ± 1.24 | 43.0 | 0.48 ± 0.02 |
| TB1-B1-Q142* | | | | | | | |
| Prob > F | | 0.77 | 0.95 | 0.83 | 0.33 | 0.87 | 0.88 |

^{a}Denotes the number of lines in each genotype class.
Values represent the average for each genotype ± the standard error.

In the Tb1-A1-W339*/Tb1-B1a, Tb1-A1a/Tb1-B1-W341* cross (Tables 18a and 18b), the double mutant had an increase in tillers per plant and reduced spikelets and flag leaf length. The TB1-A1-W339*, TB1-B1 genotype also had reduced flag leaf length. Productive heads and biomass trended upwards when mutations were present.

In the Tb1-A1-W339*/Tb1-B1a, Tb1-A1a/Tb1-B1-W341* cross (Tables 18a and 18b), the double mutant had an increase in tillers per plant and reduced spikelets and flag leaf length. The TB1-A1-W339*, TB1-B1 genotype also had reduced flag leaf length. Productive heads and biomass trended upwards when mutations were present.

TABLE 18A
Comparison of agronomic trait measurements taken on each of the four
genotypes produced in the Tb1-A1-W339*/Tb1-B1a, Tb1-A1a/Tb1-B1-W341*
cross, from field trials combined across three Montana environments.

| | | Plant | | | Productive | Flag Leaf | Flag Leaf |
|---|---|---|---|---|---|---|---|
| | | Height | Tillers | Spikelets | Heads | Length | Width |
| TB1 Genotype | N^{a} | Cm | No./plant | No./head | No./plant | cm | cm |
| TB1-A1, TB1-B1 | 16 | 69.7 ± 0.83 | 14.9 ± 0.42b | 15.0 ± 0.11 | 12.0 ± 0.42b | 20.2 ± 0.22a | 1.48 ± 0.01 |
| TB1-A1, TB1- | 18 | 69.8 ± 0.78 | 15.9 ± 0.48ab | 14.9 ± 0.88 | 12.6 ± 0.48b | 20.2 ± 0.19a | 1.48 ± 0.01 |
| B1- W341* | | | | | | | |
| TB1-A1- | 8 | 69.3 ± 1.17 | 15.8 ± 0.66ab | 14.6 ± 0.19 | 12.5 ± 0.68b | 19.4 ± 0.32b | 1.53 ± 0.02 |
| W339*, | | | | | | | |
| TB1-B1 | | | | | | | |
| TB1-A1- | 9 | 68.4 ± 1.16 | 16.7 ± 0.74a | 14.5 ± 0.13 | 13.4 ± 0.67a | 19.4 ± 0.31b | 1.47 ± 0.02 |
| W339*, | | | | | | | |
| TB1-B1- | | | | | | | |
| W341* | | | | | | | |
| Prob > F | | 0.51 | 0.04 | 0.14 | 0.008 | 0.02 | 0.23 |

^{a}Denotes the number of lines in each genotype class.
Values represent the average for each genotype ± the standard error
Means followed by different letters indicate they differ significantly (P < 0.05). T comparisons were made only following a significant (P < 0.05) F ratio.

TABLE 18B
Comparison of seed trait measurements taken on each of the four genotypes
produced in the Tb1-A1-W339*/Tb1-B1a, Tb1-A1a/Tb1-B1-W341* cross,
from field trials combined across three Montana environments.

| | | Straw | | | Seed | | |
|---|---|---|---|---|---|---|---|
| | | yield | Yield | Protein | Weight | Biomass | Harvest |
| TB1 Genotype | N^{a} | g/plant | g/plant | % | mg/seed | g/plant | Index |
| TB1-A1, TB1-B1 | 16 | 27.2 ± 0.79 | 23.8 ± 0.94 | 14.7 ± 0.14 | 49.4 ± 0.98 | 51.0 | 0.46 ± 0.01 |
| TB1-A1, TB1- | 18 | 29.8 ± 0.96 | 23.5 ± 0.96 | 14.8 ± 0.18 | 50.0 ± 0.86 | 53.2 | 0.44 ± 0.01 |
| B1-W341* | | | | | | | |
| TB1-A1-W339*, | 8 | 27.3 ± 1.08 | 23.8 ± 1.11 | 15.2 ± 0.16 | 49.5 ± 1.24 | 51.2 | 0.46 ± 0.02 |
| TB1-B1 | | | | | | | |
| TB1-A1-W339*, | 9 | 31.4 ± 2.28 | 21.9 ± 1.25 | 15.2 ± 0.23 | 51.1 ± 0.89 | 53.2 | 0.42 ± 0.02 |
| TB1-B1-W341* | | | | | | | |
| Prob > F | | 0.220 | 0.18 | 0.15 | 0.46 | 0.63 | 0.12 |

^{a}Denotes the number of lines in each genotype class.
Values represent the average for each genotype ± the standard error.

### RNA Sequencing Expression Analysis

Total RNA was extracted from a composite sample of each of the 4 TB1 genotypes in the Tb1-A1-W339*/Tb1-B1a, Tb1-A1a/Tb1-B1-Q142* cross to assess the expression of a range of genes including TB1 and genes associated with the GA (gibberellic acid) signaling, JA (jasmonic acid) signaling, ABA (abscissic acid) biosynthesis, and ABA signaling pathways. These particular pathways were chosen due to their effect on plant growth and development. Table 19 shows significant fold change of the RPKM (mean reads per kilobase of transcript, per million mapped reads) of each mutant TB1 sample versus the wild type TB1. Other related genes in the mentioned pathways were analyzed as well, with no change detected. All results were normalized to the housekeeping gene Actin. TB1-A and TB1-B were both expressed at low levels in all 4 genotypes, with TB1-A consistently higher. In the double mutant TB1-A1-W339*, TB1-B1-Q142*, TB1-B expression was decreased by 41%, while TB1-A expression was increased by 26%. In TB1-A1-W339*, TB1-B1, expression of both TB1 genes was upregulated by around 55%. In TB1-A, TB1-B1-Q142*, both were downregulated, TB1-A by 43% and TB1-B by 61%.

The JA signaling pathway contained the most genes with significantly altered level of expression of the plant growth and development-regulating genes analyzed. Downregulated in that pathway were a WRKY gene, TRITD7Bv1G060720, decreased by 55% and a JAV1 gene, TRITD7Av1G252390, decreased by 74% in the double mutant sample. In the TB1-A mutant sample, a Jasmonyl-isoleucine synthetase gene TRITD1Bv1G222110 decreased by 23%.

TABLE 19
List of genes analyzed using total RNA sequencing, with results including fold change from wild type displayed for each TB1 mutant genotype.

| | | TB1-A1-W339*, | | TB1-A1-W339*, | | TB1-A1, | | |
|---|---|---|---|---|---|---|---|---|
| | | TB1-B1-Q142* | | TB1-B1 | | TB1-B1-Q142* | | TB1-A1, |
| | | | fold | | fold | | fold | TB1-B1 |
| Protein | Gene | RPKM [symbol] | change | RPKM [symbol] | change | RPKM [symbol] | change | RPKM [symbol] |
| Actin | TRITD4Av1G220530 | 144.0 ± 0.00 | 1.00 | 144.0 ± 0.00 | 1.00 | 144.0 ± 0.00 | 1.00 | 144.0 ± 0.00 |
| TB1-A | TRITD4Av1G194310 | 1.41 ± 0.12 | 1.26 * | 1.72 ± 0.68 | 1.54 | 0.64 ± 0.06 | 0.57 ** | 1.12 ± 0.06 |
| TB1-B | TRITD4Bv1G012050 | 0.55 ± 0.12 | 0.59 | 1.44 ± 0.42 | 1.55 | 0.36 ± 0.11 | 0.39 * | 0.92 ± 0.16 |
| Gibberellin Signaling Pathway | | | | | | | | |
| Rht-A [symbol] | TRITD4Av1G194130 | 94.65 ± 12.13 | 0.96 | 101.95 ± 12.42 | 1.03 | 83.73 ± 1.47 | 0.85 * | 99.07 ± 3.79 |
| Rht-B [symbol] | TRITD4Bv1G012280 | 122.78 ± 12.84 | 1.01 | 137.82 ± 17.31 | 1.14 | 107.06 ± 5.86 | 0.88 | 121.00 ± 12.92 |
| IDD- | TRITD3Av1G032110 | 12.17 ± 1.07 | 0.87 | 14.81 ± 1.87 | 1.06 | 10.70 ± 0.17 | 0.77 * | 13.91 ± 1.02 |
| like | | | | | | | | |
| IDD- | TRITD3Av1G077250 | 0.33 ± 0.13 | 4.24 | 0.16 ± 0.00 | 2.12 * | 0.14 ± 0.01 | 1.80 | 0.08 ± 0.04 |
| like | | | | | | | | |
| BZR/ | TRITD2Av1G065030 | 30.54 ± 5.12 | 1.14 | 29.80 ± 2.09 | 1.11 | 23.52 ± 0.65 | 0.88 * | 26.74 ± 0.85 |
| BE S | | | | | | | | |
| PIF4- | TRITD2Bv1G121430 | 0.99 ± 0.29 | 0.47 * | 1.58 ± 0.37 | 0.74 | 1.18 ± 0.17 | 0.55 * | 2.13 ± 0.34 |
| like | | | | | | | | |
| Jasmonic Acid Signaling Pathway | | | | | | | | |
| Jasmonyl- | TRITD1Bv1G222110 | 4.70 ± 0.38 | 0.78 * | 4.68 ± 0.27 | 0.77 ** | 5.69 ± 0.36 | 0.94 | 6.05 ± 0.20 |
| isoleucine | | | | | | | | |
| synthetase | | | | | | | | |
| Jasmonyl- | TRITD3Av1G058120 | 2.11 ± 0.25 | 1.65 * | 2.59 ± 0.73 | 2.03 | 1.18 ± 0.11 | 0.92 | 1.28 ± 0.02 |
| isoleucine | | | | | | | | |
| synthetase | | | | | | | | |
| CO [symbol] | TRITD3Bv1G206960 | 51.90 ± 4.23 | 1.02 | 57.12 ± 9.24 | 1.12 | 42.90 ± 0.46 | 0.84 | 51.10 ± 7.26 |
| JAV1 | TRITD7Av1G252390 | 0.12 ± 0.02 | 0.26 ** | 0.58 ± 0.29 | 1.21 | 0.87 ± 0.45 | 1.81 | 0.48 ± 0.08 |
| WRKY | TRITD2Av1G267740 | 6.16 ± 1.46 | 2.07 * | 10.36 ± 2.64 | 3.47 * | 3.59 ± 0.71 | 1.20 | 2.98 ± 0.19 |
| WRKY | TRITD2Bv1G231460 | 8.75 ± 1.79 | 2.15 * | 13.89 ± 1.60 | 3.41 ** | 6.05 ± 1.77 | 1.48 | 4.07 ± 0.74 |
| WRKY | TRITD3Bv1G082570 | 1.77 ± 0.49 | 1.18 | 3.17 ± 1.65 | 2.13 | 5.82 ± 1.83 | 3.90 * | 1.49 ± 0.66 |
| WRKY | TRITD6Av1G052760 | 38.51 ± 5.11 | 1.13 | 51.45 ± 19.95 | 1.51 | 69.57 ± 12.38 | 2.04 * | 34.12 ± 11.04 |
| WRKY | TRITD7Bv1G060710 | 0.32 ± 0.03 | 0.90 | 0.75 ± 0.32 | 2.09 | 0.67 ± 0.01 | 1.86 ** | 0.36 ± 0.05 |
| WPKY | TRITD7Bv1G060720 | 0.35 ± 0.04 | 0.45 *** | 0.69 ± 0.30 | 0.91 | 0.67 ± 0.08 | 0.88 | 0.76 ± 0.04 |
| JAZ | TRITD4Av1G184160 | 0.68 ± 0.11 | 1.53 | 1.16 ± 0.63 | 2.61 | 0.78 ± 0.13 | 1.76 * | 0.45 ± 0.08 |
| JAZ | TRITD4Bv1G007870 | 12.81 ± 1.59 | 1.07 | 12.39 ± 1.32 | 1.04 | 11.65 ± 0.90 | 0.98 | 11.93 ± 1.05 |
| JAZ | TRITD0Uv1G02 [symbol] 260 | 10.95 ± 1.14 | 1.07 | 10.61 ± 1.57 | 1.04 | 10.16 ± 0.97 | 0.99 | 10.23 ± 0.93 |
| JAZ | TRITD4Bv1G172710 | 0.48 ± 0.03 | 1.21 | 0.66 ± 0.33 | 1.66 | 1.38 ± 0.30 | 3.46 * | 0.40 ± 0.24 |
| JAZ | TRITD6Av1G037770 | 26.33 ± 0.62 | 1.12 * | 24.57 ± 0.69 | 1.05 | 21.51 ± 1.57 | 0.92 | 23.44 ± 0.54 |
| Rht-A [symbol] | TRITD4Av1G194130 | 94.65 ± 12.13 | 0.96 | 101.95 ± 12.42 | 1.03 | 83.73 ± 1.47 | 0.85 * | 99.07 ± 3.79 |
| Rht-B [symbol] | TRITD4Bv1G012280 | 122.78 ± 12.84 | 1.01 | 137.82 ± 17.31 | 1.14 | 107.06 ± 5.86 | 0.88 | 121.00 ± 12.92 |
| EIN3/ | TRITD5Av1G159810 | 3.70 ± 0.25 | 0.62 * | 5.92 ± 1.05 | 0.99 | 5.19 ± 0.50 | 0.87 | 5.99 ± 0.49 |
| EIL1 | | | | | | | | |
| EIN3/ | TRITD5Bv1G150070 | 4.42 ± 0.72 | 0.67 * | 6.27 ± 1.18 | 0.95 | 6.39 ± 0.86 | 0.97 | 6.61 ± 0.76 |
| EIL1 | | | | | | | | |
| ORA59/ | TRITD4Bv1G001490 | 0.21 ± 0.06 | 0.74 | 0.31 ± 0.16 | 1.11 | 0.02 ± 0.02 | 0.06 * | 0.28 ± 0.11 |
| ERF- | | | | | | | | |
| like | | | | | | | | |
| PDF | TRITD4Bv1G184330 | 22.28 ± 3.68 | 0.94 | 17.70 ± 1.62 | 0.75 * | 18.09 ± 2.37 | 0.76 * | 23.71 ± 0.97 |
| 2.1- | | | | | | | | |
| like | | | | | | | | |
| PDF | TRITD6Bv1G028180 | 1.34 ± 0.56 | 1.78 | 0.93 ± 0.47 | 1.23 | 2.21 ± 0.50 | 2.94 * | 0.75 ± 0.23 |
| 2.1- | | | | | | | | |
| like | | | | | | | | |
| MYC- | TRITD3Bv1G076400 | 12.47 ± 1.13 | 0.84 * | 16.83 ± 2.51 | 1.13 | 18.95 ± 3.24 | 1.27 | 14.89 ± 0.23 |
| like | | | | | | | | |
| MYC- | TRITD3Bv1G148410 | 8.58 ± 1.43 | 1.00 | 20.80 ± 8.80 | 2.42 | 23.08 ± 5.28 | 2.68 * | 8.60 ± 3.43 |
| like | | | | | | | | |
| MYC- | TRITD5Bv1G166430 | 3.30 ± 1.08 | 1.15 | 1.61 ± 0.28 | 0.56 * | 3.33 ± 1.49 | 1.16 | 2.86 ± 0.26 |
| like | | | | | | | | |
| MYC- | TRITD5Bv1G166420 | 2.42 ± 0.77 | 1.00 | 1.30 ± 0.23 | 0.54 * | 2.73 ± 1.21 | 1.13 | 2.41 ± 0.35 |
| like | | | | | | | | |
| TPL | TRITD3Bv1G086210 | 44.56 ± 6.45 | 0.67 * | 65.89 ± 8.18 | 0.98 | 55.05 ± 5.01 | 0.82 | 66.96 ± 4.10 |
| TPL | TRITD7Av1G146530 | 59.46 ± 13.74 | 0.76 | 76.61 ± 8.01 | 0.98 | 66.94 ± 1.26 | 0.86 * | 77.84 ± 2.98 |
| TPL | TRITD7Bv1G106040 | 43.97 ± 9.70 | 0.78 | 49.28 ± 5.08 | 0.88 | 47.38 ± 3.85 | 0.84 * | 56.18 ± 1.43 |
| HDA19- | TRITD6Av1G081960 | 51.80 ± 3.33 | 0.80 * | 51.41 ± 2.49 | 0.80 * | 53.68 ± 6.41 | 0.83 | 64.38 ± 4.95 |
| like | | | | | | | | |
| HDA19- | TRITD7Av1G198070 | 17.17 ± 1.07 | 0.75 * | 19.41 ± 2.46 | 0.84 * | 19.85 ± 1.59 | 0.86 | 23.04 ± 1.61 |
| like | | | | | | | | |
| ABA Biosynthesis Pathway | | | | | | | | |
| NCED/ | TRITD2Bv1G125530 | 0.05 ± 0.01 | 0.87 | 0.14 ± 0.02 | 2.74 * | 0.11 ± 0.04 | 2.07 | 0.05 ± 0.03 |
| TaNCED | | | | | | | | |
| vp15 | TRITD5Av1G193780 | 0.08 ± 0.06 | 0.77 | 0.28 ± 0.08 | 2.87 * | 0.31 ± 0.17 | 3.08 | 0.10 ± 0.03 |
| ABA Signaling Pathway | | | | | | | | |
| PYR1- | TRITD4Bv1G118420 | 20.44 ± 3.23 | 1.46 | 21.36 ± 1.34 | 1.53 * | 13.70 ± 1.90 | 0.98 | 13.97 ± 1.66 |
| like | | | | | | | | |
| PYL5 | TRITD4Av1G055360 | 8.47 ± 1.92 | 1.50 | 8.29 ± 0.44 | 1.47 ** | 4.50 ± 0.43 | 0.80 | 5.64 ± 0.36 |
| TEM1 | TRITD1Av1G218480 | 1.38 ± 0.33 | 1.31 | 3.31 ± 1.29 | 3.15 | 2.49 ± 0.53 | 2.36 * | 1.05 ± 0.04 |
| ZHD15 | TRITD4Bv1G015270 | 5.57 ± 1.58 | 0.93 | 5.58 ± 0.55 | 0.93 | 3.41 ± 0.40 | 0.57 ** | 6.02 ± 0.16 |

^{a}Reads per kilobase of transcript, per million mapped reads, normalized to Actin (TRITD4Av1G220530). Mean plus and minus the standard error of three technical replicates
^{b}Rht genes are part of both the gibberellin and jasmonic acid signaling pathways.
*, **, and *** denote P values of <0.05. 0.01, and 0.001 respectively. [symbol]
indicates data missing or illegible when filed

Upregulated genes include two more WRKY genes: TRITD2Bv1G231460 increased by 241% in the TB1-A mutant, and TRITD7Bv1G060710 increased by 86% in the TB1-B mutant. In the ABA signaling pathway, TRITD4Av1G055360, a PYL5 gene was upregulated 47% in the TB1-A mutant, and a ZHD15 gene, TRITD4Bv1G015270, was downregulated 43% in the TB1-B mutant.

### Variety Screen

Forty wheat varieties were analyzed for mutations in TB1, along with controls containing known mutations. One silent mutation present in two varieties and one missense mutation predicted to have a neutral effect in one variety were found in the A genome of spring wheat. Two more spring wheat varieties were found to have the TB-B1b allele, consisting of two silent mutations and three missense mutations with predicted neutral effects, previously reported by Dixon et al. (2018). Also in the B genome, five durum varieties were found to have a missense mutation predicted to have a borderline deleterious effect. Lastly, four spring wheat varieties were found to contain a predicted deleterious missense mutation in the D genome, also identified previously by Dixon et al. (2018). For specific varieties of spring and durum wheat and the mutations found, see Table 20.

TABLE 20
List of variant TB1 alleles present in durum and
bread wheat varieties commonly grown in Montana.

| | DNA | Original | New | Provean | |
|---|---|---|---|---|---|
| Allele | Change^{a} | Codon | Codon | Score^{b} | Identified In: |
| TB1-A1-A104A | 312 | GCT | GCC | NA | ‘Alum’, ‘WB-Gunnison’ |
| TB1-A1-A314P | 940 | GCT | CCT | −0.952 | ‘SY-Valda’ |
| TB1-B1-S184G | 550 | AGC | GGC | −2.071 | ‘Riveland’, ‘Joppa’, |
| | | | | | ‘Tioga’, ‘Precision’, |
| | | | | | ‘Mountrail’ |
| TB1-B1-A271V^{c} | 812 | GCA | GTA | −0.061 | ‘McNeal’, ‘Egan’ |
| TB1-B1-L340L^{c} | 912 | TTG | TTA | NA | ‘McNeal’, ‘Egan’ |
| TB1-B1-G66G^{c} | 198 | GAG | GAA | NA | ‘McNeal’, ‘Egan’ |
| TB1-B1-S103R^{c} | 309 | AGC | AGG | −0.291 | ‘McNeal’, ‘Egan’ |
| TB1-B1-K221N^{c} | 663 | AAA | AAT | −1.112 | ‘McNeal’, ‘Egan’ |
| TB1-D1-D110Y | 328 | GAC | TAC | −7.939 | ‘Duclair’, ‘Vida, |
| | | | | | ‘Choteau’, ‘NS-Presser’ |

^{a}Nucleotide locations are relative to the coding sequence starting with the start codon.
^{b}Provean (Protein Variation Effect Analyzer) score used to predict effect: A score of −2.5 for single amino acid substitutions is predicted to be deleterious 80% of the time, with higher scores having a deleterious effect less often and lower scores more often.
^{c}These five mutations make up the TB-B1b allele reported by Dixon et al. (2018).

### Root Analysis

Root biomass, aboveground biomass, and tillers per pot were analyzed at one-month and two months post planting (Table 21a). The number of lines analyzed per genotype group differed due to excluding pots that were affected by disease or mortality. At both time points, there were no detectable differences among genotypes in either root or aboveground biomass. One month after planting, plants with TB1-A1-W339*, either with the wild type TB1-B1 or in combination with the mutant TB1-B1-Q142* allele, had 44% and 68% more tillers, respectively, than the TB1-A1, TB1-B1 wild type.

TABLE 21a
Root biomass, tillers, and aboveground biomass comparisons
by genotype on a per plant basis, one month after planting.

| | | Root | | |
|---|---|---|---|---|
| | | Biomass | Tillers | Biomass |
| | | (mean ± SE, | (mean ± SE, | (mean ± SE, |
| Genotype | n | g/plant) | #/plant) | g/plant) |
| [symbol] | 8 | 1.18 ± 0.23 [symbol] | 12.0 ± 1.03 [symbol] | 2.04 ± 0.30 [symbol] |
| [symbol] | 9 | 1.19 ± 0.22 [symbol] | 12.3 ± 0.97 [symbol] | 2.17 ± 0.28 [symbol] |
| [symbol] | 7 | 1.84 ± 0.25 [symbol] | 17.3 ± 1.10 [symbol] | 2.91 ± 0.32 [symbol] |
| [symbol] | 8 | 1.78 ± 0.23 [symbol] | 20.2 ± 1.03 [symbol] | 2.95 ± 0.30 [symbol] |

^{a}Superscript numbers indicate groups with statistical differences at the P < 0.05 level. Genotypes with the same letter mean there is no statistical difference between them.
n denotes the number of replicates (pots) measured for each genotype [symbol]
indicates data missing or illegible when filed

### Discussion

The initial greenhouse trial revealed trends of increases in number of total tillers and productive heads when TB1 mutations were present, either alone or in combination (Tables 15a and 15b). Therefore, we proceeded with field trials on F3 plants. In the first year, we found that in the Tb1-A1-W339*/Tb1-B1a, Tb1-A1a/Tb1-B1-Q142* cross, either mutation provided a tiller number increase of around 20%, while the mutations together added nearly 30% more tillers (Table 10). This agrees with previous research (Cubas et al. 1999, Xu et al. 2014, Kebrom et al. 2006, and Liu et al. 2018) indicating that mutations in TB1 cause a bushier, more branched plant architecture in a variety of species. However, the current research was the first demonstration that TB1 null mutations could be used to increase tiller number in wheat.

Continuing field trials in two locations for the second year, it was possible to examine GxE interactions. We found that only tiller number in the Tb1-A1-W339*/Tb1-B1a, Tb1-A1a/Tb1-B1-Q142* cross differed significantly by environment. This interaction was further examined, and it was found that the tiller number in the first year was the only significant difference between environments (Table 16). The remaining response variables were analyzed across environments. Spikelet numbers were reduced in the double mutant TB1-A1-W339*, TB1-B1-Q142*, while flag leaves were shorter in both it and the TB1-A1-W339*, TB1-B1 single mutant (Tables 17a and 17b). The reduction in spikelet number is in contrast to the findings of Dixon et al. (2018), who observed that paired spikelets were present when TB1 was overexpressed. As expected, based on greenhouse trials, mutant genotypes had a trend of increased tillers and productive heads. There was also a trend of increased height with TB1 null mutations, which agrees with Dixon et al. (2020) and Hubbard et al. (2002) who both found that TB1 mutations resulted in taller plants.

In the other cross, the double mutant TB1-A1-W339*, TB1-B1-W341* had increased tillers along with reduced spikelets per head, and it, along with the single mutant TB1-A1-W339*, TB1-B1, had reduced flag leaf length. All mutations alone displayed a trend towards increased biomass, more dramatically so when combined. Gene expression analysis revealed few trends. A list of GA, JA, and ABA signaling pathway genes was analyzed along with TB1-A and TB1-B. Expression in the double mutant genotype was increased for TB1-A, which does not fit the expectations based on Dixon et al. (2018)'s findings that increased expression of TB1-A decreased tiller number. In this project, we used stop mutations that were found to increase tiller number. We were interested to see if the coding sequence change had any effect on RNA expression levels of TB1 and other genes associated with growth regulating hormones. TB1-B was reduced in the double mutant which did agree with Dixon et al. (2018). In the single mutant genotypes, TB1-A and TB1-B were both upregulated in the TB1-A mutant, and both downregulated in the TB1-B mutant. In the plant growth regulatory genes, expression varied considerably. There were no trends of a group of genes or even a single gene that were consistently up or downregulated whenever mutations were present. Our results disagree with those of Dong et al. (2019) who found that in TB1 mutant plants, all but two ABA biosynthesis and signaling genes were significantly downregulated, and all JA biosynthesis and signaling genes were significantly downregulated.

To determine if TB1 mutant genotypes already exist amongst Montana top grown wheat varieties, a screen was conducted in which the entire TB1 gene was sequenced and examined for mutations in 40 different varieties. In all, 9 different mutations were identified. The allele known as TB1-B1b, which consists of a suite of five mutations, was identified in McNeal and Egan spring wheat. This allele was previously identified by Dixon et al. (2018). Also identified by Dixon et al. (2018) research was the TB1-D1-D110Y mutation, which we found in four spring wheat varieties commonly grown in Montana. We also identified three mutations in durum wheat that were identified in our examination of previous literature. This indicates that there is natural allelic variation in both hexaploid and tetraploid wheat.

Along with examining the effects of TB1 mutations on field plant characteristics and on gene expression, a below ground study on roots was conducted. Aboveground biomass, root biomass, and tiller counts were taken on the experimental plants. At one-month post-planting, the TB1-A1-W339* allele, either with the wild type TB1-B1 or in combination with the mutant TB1-B1-Q142* allele, was associated with a large increase in tillering, while in the other cross there were only increased tillers when both mutations were present. By two months post-planting, only the double mutant had increased tillers. We did not see any statistically significant differences in above- or belowground biomass, which contradicts the findings of Gaudin et al. (2014), who found that in maize plants with a TB1 mutation, increased tillering was strongly correlated with an increase in root biomass proportional to the increase in plant biomass. One factor that complicated this experiment was the tendency of the pots to hold water, which led to the development of fungal infections in the growth media and rotted roots. Overall, the plants did not appear healthy and robust compared to either the previous greenhouse trial or any of the field experiments. A different method of growing plants for root examinations may yield different and more representative results.

Each of the TB1 experiments was intended to detect differences in tiller number or other characteristics due to TB1 allelic differences. Although the presence of TB1 mutations appeared to increase tiller number and plant height in the 2019 field trials, once two more environments/years were added, those results were less noticeable. Root biomass was not shown to be affected by TB1 mutations, but that experiment may yield different results under more favorable growing conditions.

Analyzing gene expression with RNA sequencing revealed that some genes which regulate plant growth hormones are upregulated, some downregulated, and many are unaffected. When taken together, these results indicate that TB1 mutations in semi-dwarf durum did not have the intended impact of increasing yield through increased tiller number. However, the trends do support further research on TB1 mutations' impacts on wheat, including the potential to increase biomass of forage-type winter wheat and to examine the impacts in hexaploid spring wheat as well. The screen of allelic variation in TB1 in currently growth Montana varieties show that natural variation does exist and may be a contributor to these varieties' success.

SEQUENCE ID INFORMATION

>Rht-A1a (SEQ ID NO 001)
Type: DNA
Organism: Triticum turgidum spp. Durum
Length: 1863 base pairs
Definition: The Rht-Ala Wild Type Allele of Rht-A1
ATGAAGCGCGAGTACCAGGACGCCGGCGGGAGCGGTGGCGGGGGCGGCATGGGCTCGTCTGAGGACAAGATGATG
GTGTCGGCGGCGGCGGGGGAGGGGGAGGAGGTGGACGAGCTGCTGGCGGCGCTCGGGTACAAGGTGCGGGCCTCC
GACATGGCGGACGTGGCGCAGAAGCTGGAGCAGCTGGAGATGGCCATGGGGATGGGCGGCGTGGGCGCCGGCGCC
GCCCCCGACGACAGCTTCGCCACCCACCTCGCCACGGACACCGTGCACTACAACCCCACCGACCTCTCCTCCTGG
GTCGAGAGCATGCTGTCGGAGCTCAACGCGCCGCCGCCGCCCCTCCCGCCCGCCCCGCAGCAGCTCAACGCCTCC
ACCTCCTCCACCGTCACGGGCGGTGGGTACTTCGATCTCCCGCCCTCGGTTGACTCCTCCTGCAGCACCTACGCC
CTGCGGCCGATCCCCTCCCCGGCCGGCGCCGTCGGGCCGGCCGACCTGTCCGCCGACTCCGTGCGGGACCCCAAG
CGGATGCGCACTGGCGGGAGCAGCACCTCGTCGTCGTCATCCTCCTCGTCCTCTCTCGGTGGGGGCGCCAGGAGC
TCTGTGGTGGAGGCTGCTCCGCCGGTCGCGGCCGGGGCCAACGCGCCCGCGCTGCCGGTCGTCGTGGTCGACACG
CAGGAGGCCGGGATTCGGCTGGTGCACGCGCTGCTGGCCTGCGCGGAGGCCGTGCAGCAGGAGAACTTCTCTGCC
GCGGAGGCGCTGGTGAAGCAGATACCCTTGCTGGCCGCGTCCCAGGGCGGCGCGATGCGCAAGGTCGCCGCCTAC
TTCGGCGAGGCCCTCGCCCGCCGCGTCTTCCGCTTCCGCCCGCAGCCGGACAGCTCCCTCCTCGACGCCGCCTTC
GCCGACCTCCTCCACGCGCACTTCTACGAGTCCTGCCCCTACCTCAAGTTCGCCCACTTCACCGCCAACCAGGCC
ATCCTGGAGGCCTTCGCCGGCTGCCGCCGCGTGCACGTCGTCGACTTCGGCATCAAGCAGGGGATGCAGTGGCCC
GCCCTCCTCCAGGCCCTCGCGCTCCGTCCCGGCGGCCCTCCCTCGTTCCGCCTCACCGGCGTCGGCCCCCCGCAG
CCGGACGAGACCGACGCCTTGCAGCAGGTGGGCTGGAAGCTCGCCCAGTTCGCGCACACCATCCGCGTCGACTTC
CAGTACCGCGGCCTCGTCGCCGCCACGCTCGCGGACCTGGAGCCATTCATGCTGCAGCCGGAGGGCGAGGAGGAC
CCGAACGAGGAGCCCGAGGTAATCGCCGTCAACTCGGTCTTCGAGATGCACCGGCTGCTCGCGCAGCCCGGCGCC
CTGGAGAAGGTCCTGGGCACCGTGCGCGCCGTGCGGCCGAGGATCGTCACCGTGGTGGAGCAGGAGGCCAACCAC
AACTCCGGCACATTCCTGGACCGCTTCACCGAGTCTCTGCACTACTACTCCACCATGTTCGATTCCCTCGAGGGC
GGCAGCTCCGGCGGCCCATCCGAAGTCTCATCGGGGGCTGCCGCTGCTCCTGCCGCCGCCGGCACGGACCAGGTC
ATGTCCGAGGTGTACCTCGGCCGGCAGATCTGCAACGTGGTGGCCTGCGAGGGGGCGGAGCGCACAGAGCGCCAC
GAGACGCTGGGGCAGTGGCGGAACCGGCTGGGCAACGCCGGGTTCGAGACCGTGCACCTGGGCTCCAATGCCTAC
AAGCAGGCGAGCACGCTGCTGGCCCTATTCGCCGGCGGCGACGGGTACAAGGTGGAGGAGAAGGAGGGCTGCCTG
ACTCTCGGGTGGCACACGCGCCCGCTGATCGCCACCTCGGCATGGCGCCTGGCCGCGCCGTGA
>Rht-A1-MDIV169-L358F (SEQ ID NO 002)
Type: DNA
Organism: Triticum turgidum spp. Durum
Length: 1863 base pairs
Definition: Mutant Allele of Rht-A1a
Location: Mutation at the 1072 base pair
Other Information: Base change from C to T resulting in amino acid change
L358F
ATGAAGCGCGAGTACCAGGACGCCGGCGGGAGCGGTGGCGGGGGCGGCATGGGCTCGTCTGAGGACAAGATGATG
GTGTCGGCGGCGGCGGGGGAGGGGGAGGAGGTGGACGAGCTGCTGGCGGCGCTCGGGTACAAGGTGCGGGCCTCC
GACATGGCGGACGTGGCGCAGAAGCTGGAGCAGCTGGAGATGGCCATGGGGATGGGCGGCGTGGGCGCCGGCGCC
GCCCCCGACGACAGCTTCGCCACCCACCTCGCCACGGACACCGTGCACTACAACCCCACCGACCTCTCCTCCTGG
GTCGAGAGCATGCTGTCGGAGCTCAACGCGCCGCCGCCGCCCCTCCCGCCCGCCCCGCAGCAGCTCAACGCCTCC
ACCTCCTCCACCGTCACGGGCGGTGGGTACTTCGATCTCCCGCCCTCGGTTGACTCCTCCTGCAGCACCTACGCC
CTGCGGCCGATCCCCTCCCCGGCCGGCGCCGTCGGGCCGGCCGACCTGTCCGCCGACTCCGTGCGGGACCCCAAG
CGGATGCGCACTGGCGGGAGCAGCACCTCGTCGTCGTCATCCTCCTCGTCCTCTCTCGGTGGGGGCGCCAGGAGC
TCTGTGGTGGAGGCTGCTCCGCCGGTCGCGGCCGGGGCCAACGCGCCCGCGCTGCCGGTCGTCGTGGTCGACACG
CAGGAGGCCGGGATTCGGCTGGTGCACGCGCTGCTGGCCTGCGCGGAGGCCGTGCAGCAGGAGAACTTCTCTGCC
GCGGAGGCGCTGGTGAAGCAGATACCCTTGCTGGCCGCGTCCCAGGGCGGCGCGATGCGCAAGGTCGCCGCCTAC
TTCGGCGAGGCCCTCGCCCGCCGCGTCTTCCGCTTCCGCCCGCAGCCGGACAGCTCCCTCCTCGACGCCGCCTTC
GCCGACCTCCTCCACGCGCACTTCTACGAGTCCTGCCCCTACCTCAAGTTCGCCCACTTCACCGCCAACCAGGCC
ATCCTGGAGGCCTTCGCCGGCTGCCGCCGCGTGCACGTCGTCGACTTCGGCATCAAGCAGGGGATGCAGTGGCCC
GCCCTCCTCCAGGCCCTCGCGCTCCGTCCCGGCGGCCCTCCCTCGTTCCGCCTCACCGGCGTCGGCCCCCCGCAG
CCGGACGAGACCGACGCCTTGCAGCAGGTGGGCTGGAAGCTCGCCCAGTTCGCGCACACCATCCGCGTCGACTTC
CAGTACCGCGGCCTCGTCGCCGCCACGCTCGCGGACCTGGAGCCATTCATGCTGCAGCCGGAGGGCGAGGAGGAC
CCGAACGAGGAGCCCGAGGTAATCGCCGTCAACTCGGTCTTCGAGATGCACCGGCTGCTCGCGCAGCCCGGCGCC
CTGGAGAAGGTCCTGGGCACCGTGCGCGCCGTGCGGCCGAGGATCGTCACCGTGGTGGAGCAGGAGGCCAACCAC
AACTCCGGCACATTCCTGGACCGCTTCACCGAGTCTCTGCACTACTACTCCACCATGTTCGATTCCCTCGAGGGC
GGCAGCTCCGGCGGCCCATCCGAAGTCTCATCGGGGGCTGCCGCTGCTCCTGCCGCCGCCGGCACGGACCAGGTC
ATGTCCGAGGTGTACCTCGGCCGGCAGATCTGCAACGTGGTGGCCTGCGAGGGGGCGGAGCGCACAGAGCGCCAC
GAGACGCTGGGGCAGTGGCGGAACCGGCTGGGCAACGCCGGGTTCGAGACCGTGCACCTGGGCTCCAATGCCTAC
AAGCAGGCGAGCACGCTGCTGGCCCTATTCGCCGGCGGCGACGGGTACAAGGTGGAGGAGAAGGAGGGCTGCCTG
ACTCTCGGGTGGCACACGCGCCCGCTGATCGCCACCTCGGCATGGCGCCTGGCCGCGCCGTGA
>Rht-A1-MDIV4-S50F (SEQ ID NO 003)
Type: DNA
Organism: Triticum turgidum spp. Durum
Length: 1863 base pairs
Definition: Mutant Allele of Rht-Ala
Location: Mutation at the 149 base pair
Other Information: Base change from C to T resulting in amino acid change
S50F
ATGAAGCGCGAGTACCAGGACGCCGGCGGGAGCGGTGGCGGGGGCGGCATGGGCTCGTCTGAGGACAAGATGATG
GTGTCGGCGGCGGCGGGGGAGGGGGAGGAGGTGGACGAGCTGCTGGCGGCGCTCGGGTACAAGGTGCGGGCCTTC
GACATGGCGGACGTGGCGCAGAAGCTGGAGCAGCTGGAGATGGCCATGGGGATGGGCGGCGTGGGCGCCGGCGCC
GCCCCCGACGACAGCTTCGCCACCCACCTCGCCACGGACACCGTGCACTACAACCCCACCGACCTCTCCTCCTGG
GTCGAGAGCATGCTGTCGGAGCTCAACGCGCCGCCGCCGCCCCTCCCGCCCGCCCCGCAGCAGCTCAACGCCTCC
ACCTCCTCCACCGTCACGGGCGGTGGGTACTTCGATCTCCCGCCCTCGGTTGACTCCTCCTGCAGCACCTACGCC
CTGCGGCCGATCCCCTCCCCGGCCGGCGCCGTCGGGCCGGCCGACCTGTCCGCCGACTCCGTGCGGGACCCCAAG
CGGATGCGCACTGGCGGGAGCAGCACCTCGTCGTCGTCATCCTCCTCGTCCTCTCTCGGTGGGGGCGCCAGGAGC
TCTGTGGTGGAGGCTGCTCCGCCGGTCGCGGCCGGGGCCAACGCGCCCGCGCTGCCGGTCGTCGTGGTCGACACG
CAGGAGGCCGGGATTCGGCTGGTGCACGCGCTGCTGGCCTGCGCGGAGGCCGTGCAGCAGGAGAACTTCTCTGCC
GCGGAGGCGCTGGTGAAGCAGATACCCTTGCTGGCCGCGTCCCAGGGCGGCGCGATGCGCAAGGTCGCCGCCTAC
TTCGGCGAGGCCCTCGCCCGCCGCGTCTTCCGCTTCCGCCCGCAGCCGGACAGCTCCCTCCTCGACGCCGCCTTC
GCCGACCTCCTCCACGCGCACTTCTACGAGTCCTGCCCCTACCTCAAGTTCGCCCACTTCACCGCCAACCAGGCC
ATCCTGGAGGCCTTCGCCGGCTGCCGCCGCGTGCACGTCGTCGACTTCGGCATCAAGCAGGGGATGCAGTGGCCC
GCCCTCCTCCAGGCCCTCGCGCTCCGTCCCGGCGGCCCTCCCTCGTTCCGCCTCACCGGCGTCGGCCCCCCGCAG
CCGGACGAGACCGACGCCTTGCAGCAGGTGGGCTGGAAGCTCGCCCAGTTCGCGCACACCATCCGCGTCGACTTC
CAGTACCGCGGCCTCGTCGCCGCCACGCTCGCGGACCTGGAGCCATTCATGCTGCAGCCGGAGGGCGAGGAGGAC
CCGAACGAGGAGCCCGAGGTAATCGCCGTCAACTCGGTCTTCGAGATGCACCGGCTGCTCGCGCAGCCCGGCGCC
CTGGAGAAGGTCCTGGGCACCGTGCGCGCCGTGCGGCCGAGGATCGTCACCGTGGTGGAGCAGGAGGCCAACCAC
AACTCCGGCACATTCCTGGACCGCTTCACCGAGTCTCTGCACTACTACTCCACCATGTTCGATTCCCTCGAGGGC
GGCAGCTCCGGCGGCCCATCCGAAGTCTCATCGGGGGCTGCCGCTGCTCCTGCCGCCGCCGGCACGGACCAGGTC
ATGTCCGAGGTGTACCTCGGCCGGCAGATCTGCAACGTGGTGGCCTGCGAGGGGGCGGAGCGCACAGAGCGCCAC
GAGACGCTGGGGCAGTGGCGGAACCGGCTGGGCAACGCCGGGTTCGAGACCGTGCACCTGGGCTCCAATGCCTAC
AAGCAGGCGAGCACGCTGCTGGCCCTATTCGCCGGCGGCGACGGGTACAAGGTGGAGGAGAAGGAGGGCTGCCTG
ACTCTCGGGTGGCACACGCGCCCGCTGATCGCCACCTCGGCATGGCGCCTGGCCGCGCCGTGA
>Rht-B1a (SEQ ID NO 004)
Type: DNA
Organism: Triticum turgidum spp. Durum
Length: 1866 base pairs
Definition: The Rht-B1a Wild Type Allele of Rht-B1
ATGAAGCGCGAGTACCAGGACGCCGGAGGGAGCGGCGGTGGCGGGGGAGGCATGGGCTCGTCCGAGGACAAGATG
ATGGTGTCGGGGTCGGCGGCGGCGGGGGAGGGGGAGGAGGTGGACGAGCTGCTGGCGGCGCTCGGGTACAAGGTG
CGGGCGTCCGACATGGCGGACGTGGCGCAGAAGCTGGAGCAGCTGGAGATGGCCATGGGGATGGGCGGCGTGGGC
GCCGGCGCCGCGCCCGACGACAGCTTCGCCACCCACCTCGCCACGGACACCGTGCACTACAACCCCACCGACCTC
TCCTCCTGGGTCGAGAGCATGCTGTCGGAGCTCAACGCGCCGCCGCCGCCCCTCCCGCCCGCCCCGCAGCTCAAC
GCCTCCACCTCCTCCACCGTCACCGGCGGCGGGTACTTCGATCTCCCGCCCTCCGTCGACTCCTCCTGCAGCACC
TACGCGCTGCGGCCGATCCCGTCCCCGGCCGTCGCGCCGGCCGACCTCTCCGCCGACTCCGTCGTGCGGGATCCC
AAGCGGATGCGCACTGGCGGCAGCAGCACCTCGTCGTCATCCTCATCGTCCTCTCTCGGCGGTGGCGGCGCCAGG
AGCTCTGTGGTGGAGGCTGCCCCGCCGGTGGCCGCCGCGGCCGGTGCGCCCGCGCTGCCGGTCGTCGTGGTCGAC
ACGCAGGAGGCCGGGATTCGGCTGGTGCACGCGCTGCTGGCGTGCGCAGAGGCCGTGCAGCAGGAGAACTTCTCT
GCCGCGGAGGCGCTGGTGAAGCAGATACCCTTGCTGGCCGCGTCCCAGGGCGGCGCCATGCGCAAGGTCGCCGCC
TACTTCGGCGAGGCCCTCGCCCGCCGCGTCTTCCGCTTCCGCCCGCAGCCGGACAGCTCCCTCCTCGACGCCGCC
TTCGCCGACCTCCTCCACGCGCACTTCTACGAGTCCTGCCCCTACCTCAAGTTCGCCCACTTCACCGCCAACCAG
GCCATCCTGGAGGCGTTCGCCGGCTGCCGCCGCGTGCACGTCGTCGACTTCGGCATCAAGCAGGGGATGCAGTGG
CCCGCCCTTCTCCAGGCCCTGGCGCTCCGTCCCGGCGGCCCTCCCTCGTTCCGCCTCACCGGCGTCGGCCCCCCG
CAGCCGGACGAGACCGACGCCTTGCAGCAGGTGGGCTGGAAGCTCGCCCAGTTCGCGCACACCATCCGCGTCGAC
TTCCAGTACCGCGGCCTCGTCGCCGCCACGCTCGCGGACCTGGAGCCGTTCATGCTGCAGCCGGAGGGCGAGGAG
GACCCGAACGAGGAGCCCGAGGTAATCGCCGTCAACTCGGTCTTCGAGATGCACCGGCTGCTCGCGCAGCCCGGC
GCCCTGGAGAAGGTCCTGGGCACCGTGCGCGCCGTGCGGCCGAGGATCGTCACCGTGGTGGAGCAGGAGGCGAAC
CACAACTCCGGCACATTCCTGGACCGCTTCACCGAGTCCCTGCACTACTACTCCACCATGTTCGATTCTCTGGAG
GGCGGCAGCTCCGGCGGCCCATCCGAAGTCTCATCTGGGGCGGCTGCTGCTCCTGCCGCCGCCGGCACGGACCAG
GTCATGTCCGAGGTGTACCTCGGCCGGCAGATCTGCAACGTGGTGGCCTGCGAGGGGGCGGAGCGCACAGAGCGG
CACGAGACCCTGGGGCAGTGGCGGAACCGCCTCGGCAACGCCGGGTTCGAGACCGTCCACCTGGGCTCCAATGCC
TACAAGCAGGCGAGCACGCTGCTGGCGCTCTTCGCAGGCGGCGACGGGTACAAGGTGGAGGAGAAGGAGGGCTGC
CTGACGCTGGGGTGGCACACGCGCCCGCTGATCGCCACCTCCGCATGGCGCCTGGCCGCGCCGTGA
>Rht-B1b-Kronos-E529K (SEQ ID NO 005)
Type: DNA
Organism: Triticum turgidum spp. Durum
Length: 1866 base pairs
Definition: Mutant Allele of Rht-B1b
Location: Mutation at the 1585 base pair
Other Information: Base change from C to T resulting in amino acid change
E529K
ATGAAGCGCGAGTACCAGGACGCCGGAGGGAGCGGCGGTGGCGGGGGAGGCATGGGCTCGTCCGAGGACAAGATG
ATGGTGTCGGGGTCGGCGGCGGCGGGGGAGGGGGAGGAGGTGGACGAGCTGCTGGCGGCGCTCGGGTACAAGGTG
CGGGCGTCCGACATGGCGGACGTGGCGCAGAAGCTGGAGTAGCTGGAGATGGCCATGGGGATGGGCGGCGTGGGC
GCCGGCGCCGCGCCCGACGACAGCTTCGCCACCCACCTCGCCACGGACACCGTGCACTACAACCCCACCGACCTC
TCCTCCTGGGTCGAGAGCATGCTGTCGGAGCTCAACGCGCCGCCGCCGCCCCTCCCGCCCGCCCCGCAGCTCAAC
GCCTCCACCTCCTCCACCGTCACCGGCGGCGGGTACTTCGATCTCCCGCCCTCCGTCGACTCCTCCTGCAGCACC
TACGCGCTGCGGCCGATCCCGTCCCCGGCCGTCGCGCCGGCCGACCTCTCCGCCGACTCCGTCGTGCGGGATCCC
AAGCGGATGCGCACTGGCGGCAGCAGCACCTCGTCGTCATCCTCATCGTCCTCTCTCGGCGGTGGCGGCGCCAGG
AGCTCTGTGGTGGAGGCTGCCCCGCCGGTGGCCGCCGCGGCCGGTGCGCCCGCGCTGCCGGTCGTCGTGGTCGAC
ACGCAGGAGGCCGGGATTCGGCTGGTGCACGCGCTGCTGGCGTGCGCAGAGGCCGTGCAGCAGGAGAACTTCTCT
GCCGCGGAGGCGCTGGTGAAGCAGATACCCTTGCTGGCCGCGTCCCAGGGCGGCGCCATGCGCAAGGTCGCCGCC
TACTTCGGCGAGGCCCTCGCCCGCCGCGTCTTCCGCTTCCGCCCGCAGCCGGACAGCTCCCTCCTCGACGCCGCC
TTCGCCGACCTCCTCCACGCGCACTTCTACGAGTCCTGCCCCTACCTCAAGTTCGCCCACTTCACCGCCAACCAG
GCCATCCTGGAGGCGTTCGCCGGCTGCCGCCGCGTGCACGTCGTCGACTTCGGCATCAAGCAGGGGATGCAGTGG
CCCGCCCTTCTCCAGGCCCTGGCGCTCCGTCCCGGCGGCCCTCCCTCGTTCCGCCTCACCGGCGTCGGCCCCCCG
CAGCCGGACGAGACCGACGCCTTGCAGCAGGTGGGCTGGAAGCTCGCCCAGTTCGCGCACACCATCCGCGTCGAC
TTCCAGTACCGCGGCCTCGTCGCCGCCACGCTCGCGGACCTGGAGCCGTTCATGCTGCAGCCGGAGGGCGAGGAG
GACCCGAACGAGGAGCCCGAGGTAATCGCCGTCAACTCGGTCTTCGAGATGCACCGGCTGCTCGCGCAGCCCGGC
GCCCTGGAGAAGGTCCTGGGCACCGTGCGCGCCGTGCGGCCGAGGATCGTCACCGTGGTGGAGCAGGAGGCGAAC
CACAACTCCGGCACATTCCTGGACCGCTTCACCGAGTCCCTGCACTACTACTCCACCATGTTCGATTCTCTGGAG
GGCGGCAGCTCCGGCGGCCCATCCGAAGTCTCATCTGGGGCGGCTGCTGCTCCTGCCGCCGCCGGCACGGACCAG
GTCATGTCCAAGGTGTACCTCGGCCGGCAGATCTGCAACGTGGTGGCCTGCGAGGGGGCGGAGCGCACAGAGCGG
CACGAGACCCTGGGGCAGTGGCGGAACCGCCTCGGCAACGCCGGGTTCGAGACCGTCCACCTGGGCTCCAATGCC
TACAAGCAGGCGAGCACGCTGCTGGCGCTCTTCGCAGGCGGCGACGGGTACAAGGTGGAGGAGAAGGAGGGCTGC
CTGACGCTGGGGTGGCACACGCGCCCGCTGATCGCCACCTCCGCATGGCGCCTGGCCGCGCCGTGA
>TaTB1-4A-Wildtype (SEQ ID NO 006)
Type: DNA
Organism: Triticum turgidum spp. Durum
Length: 1059 base pairs
Definition: The TaTB1-4A Wild Type Allele of TaTB1-4A
ATGTTTCCTTTCTATGATTCCCCGAGCCCCATGGACTTACCCCTTTACCAACAGCTGCAGATCAGCCCTCCCTCG
CCAAAGGCGCCGGATCACCAATCCTTGCTCTACTACCATTCCTCCCCTGCATTCGCCGCCGACGCCTTCCACCAC
AGCTACCTCTGTGCCGGCGCCGCGACGCCGCCCGCGGCCGAGATCGACAACCAGCCGCCACCGGAGCTGCTGCTG
ATGGATCAGGCGCCAGCGCCAAGGGCAGACGGTGTTGGAACCGCACAAGGCCTGCACGGCGGTGGAGGCCTCGAC
AGCGCGGCGGCTGCCGCGGCCAGGAAAGACCGGCACAGCAAGATATGCACCGCCGGCGGGATGAGGGACCGGCGG
ATGCGGCTGTCCCTCGACGTCGCCCGCAAGTTCTTCGCGCTCCAGGACATGCTCGGCTTCGACAAGGCCAGCAAG
ACGGTGCAATGGCTCCTCAATACGTCAAAGGGCGCCATCAGGGAGGTCATGACTGACGAGGCGTCCTCCGACTGC
GAGGAGGACGGCTCCAGCAGCCTCTCCGTCGTCGACGGCAAGCACAAGCCGCCAGGGACGGAGGCTGGAGGTGGT
GATCACGGCGAGGGGAAGAAGCCGATGCCAAGGGCAGCTAGAAGAGCACCCGCCAATCCAAAGCCGCAAAGGAAA
TTGGCCAGTGCGCACCCGATCCCCGACAAGGAGTCGAGGACGAAGGCGCGGGAGAGGGCCAGGGAGCGGACGCGG
GAAAAGAACCGGATGCGATGGGTTACGCTCGCGTCTACGATTAACATCGAGCCGGCAGCCACCGGCATGGCGGCG
GCGAGGGCGGATGAGTTGATCACGAGGCCGCACAATTTTATCAATCGCTCCTCGTCCATGAACACAGCAAGCGCT
GAATTGGAGGAGGGGTGCTCGTCGTCCATGCCGAGCGAAGCTATCGTGGCTGGCTTCGGCAATGGAGGGTACGGA
AGCAGCGGCAACTACTACCAGTATCAGCTGGAGCAGCAATGGGAGCTCGCTGGAGTGGTGTTTCCCAACTCGCAG
CCCTACTGA
>TaTB1-4A-Kronos944-STOP (SEQ ID NO 007)
Type: DNA
Organism: Triticum turgidum spp. Durum
Length: 1059 base pairs
Definition: Mutant Allele of TaTB1-4A
Location: Mutation at the 1016 base pair
ATGTTTCCTTTCTATGATTCCCCGAGCCCCATGGACTTACCCCTTTACCAACAGCTGCAGATCAGCCCTCCCTCG
CCAAAGGCGCCGGATCACCAATCCTTGCTCTACTACCATTCCTCCCCTGCATTCGCCGCCGACGCCTTCCACCAC
AGCTACCTCTGTGCCGGCGCCGCGACGCCGCCCGCGGCCGAGATCGACAACCAGCCGCCACCGGAGCTGCTGCTG
ATGGATCAGGCGCCAGCGCCAAGGGCAGACGGTGTTGGAACCGCACAAGGCCTGCACGGCGGTGGAGGCCTCGAC
AGCGCGGCGGCTGCCGCGGCCAGGAAAGACCGGCACAGCAAGATATGCACCGCCGGCGGGATGAGGGACCGGCGG
ATGCGGCTGTCCCTCGACGTCGCCCGCAAGTTCTTCGCGCTCCAGGACATGCTCGGCTTCGACAAGGCCAGCAAG
ACGGTGCAATGGCTCCTCAATACGTCAAAGGGCGCCATCAGGGAGGTCATGACTGACGAGGCGTCCTCCGACTGC
GAGGAGGACGGCTCCAGCAGCCTCTCCGTCGTCGACGGCAAGCACAAGCCGCCAGGGACGGAGGCTGGAGGTGGT
GATCACGGCGAGGGGAAGAAGCCGATGCCAAGGGCAGCTAGAAGAGCACCCGCCAATCCAAAGCCGCAAAGGAAA
TTGGCCAGTGCGCACCCGATCCCCGACAAGGAGTCGAGGACGAAGGCGCGGGAGAGGGCCAGGGAGCGGACGCGG
GAAAAGAACCGGATGCGATGGGTTACGCTCGCGTCTACGATTAACATCGAGCCGGCAGCCACCGGCATGGCGGCG
GCGAGGGCGGATGAGTTGATCACGAGGCCGCACAATTTTATCAATCGCTCCTCGTCCATGAACACAGCAAGCGCT
GAATTGGAGGAGGGGTGCTCGTCGTCCATGCCGAGCGAAGCTATCGTGGCTGGCTTCGGCAATGGAGGGTACGGA
AGCAGCGGCAACTACTACCAGTATCAGCTGGAGCAGCAATAGGAGCTCGCTGGAGTGGTGTTTCCCAACTCGCAG
CCCTACTGA
>TaTB1-4B-Wildtype (SEQ ID NO 008)
Type: DNA
Organism: Triticum turgidum spp. Durum
Length: 1065 base pairs
Definition: The TaTB1-4B Wild Type Allele of TaTB1-4B
ATGTTTCCTTTCTATGATTCCCCTAGCCCCATGGACTTGCCCCTTTACCAACAGCTGCAGATCAGCCCTCCCTCG
CCAAAGGCGCCGGATCACCAATCCTTGCTCTACTACCATTCCTCCCCTGCATTTGCCGCCGACTCCTTCCACCAC
AGCTACCTCTGTGCCGGTGCCGCGACGCCCACGCCGCCCGCGGCCGAGATCGACAACCAGTCGCCGCCGGAGCTG
CTGCTGATGGATCAGGCTCCGGCGCCAAGGGCAGACGGTGTTGGAACCGCACAAGGCCTGCACGGCGGTGGAGGC
CTCGACAGCGCGGCGGCCGCCGCGGCCAGGAAAGACCGGCACAGCAAGATATGCACCGCCGGCGGGATGAGGGAC
CGGCGGATGCGGCTCTCCCTCGACGTCGCCCGCAAGTTCTTCGCGCTCCAGGACATGCTCGGCTTCGACAAGGCC
AGCAAGACGGTGCAATGGCTCCTCAATACGTCAAAGGGCGCCATCAGGGAGGTCATGACTGACGAGGCGTCCTCC
GACTGCGAGGAGGACGGCTCCAGCAGCCTCTCCGTCGTCGACGGCAAGCACAAGCCGCCAGGGACGGAGGCTGAA
GGTGGTGATCACGGCGAGGGGAAGAAGCAGGTGCCAAGGGCAGCCAGAAGGGCACCCGCCAAACCAAAGCCGCAA
AGGAAATTGGCCAGTGCGCACCCGATCCCCGACAAGGAGTCGAGGACGAAGGCGCGGGAGAGGGCGAGGGAGCGG
ACTAGGGAAAAGAACCGGATGCGATGGGTGACGCTCGCGTCCACAATTAACATCGAGCCGGCAGCCACCGGCATG
GCGGCGGCGAGGGCGGACGAGTTCGTCACGAGGCCGAACAATTTGATCAGTCGCTCCTCGTCCATGAACACAGCA
AGAGCTGAATTGGAGGAGGGGTGCTCGTCGTCCATGCCGAGCGAAGCGATCATGGCTGGCTTCGGCAATGGAGGA
TACGGAAGCAGCGGTAACTACTACCAGTATCAGCTGGAGCAGCAATGGGAGCTCGGTGGAGTGGTGTTTCCCAAC
TCCCAGCCCTACTGA
>TaTB1-4B-Kronos562-STOP (SEQ ID NO 009)
Type: DNA
Organism: Triticum turgidum spp. Durum
Length: 1065 base pairs
Definition: Mutant Allele of TaTB1-4B
Location: Mutation at the 1023 base pair
ATGTTTCCTTTCTATGATTCCCCTAGCCCCATGGACTTGCCCCTTTACCAACAGCTGCAGATCAGCCCTCCCTCG
CCAAAGGCGCCGGATCACCAATCCTTGCTCTACTACCATTCCTCCCCTGCATTTGCCGCCGACTCCTTCCACCAC
AGCTACCTCTGTGCCGGTGCCGCGACGCCCACGCCGCCCGCGGCCGAGATCGACAACCAGTCGCCGCCGGAGCTG
CTGCTGATGGATCAGGCTCCGGCGCCAAGGGCAGACGGTGTTGGAACCGCACAAGGCCTGCACGGCGGTGGAGGC
CTCGACAGCGCGGCGGCCGCCGCGGCCAGGAAAGACCGGCACAGCAAGATATGCACCGCCGGCGGGATGAGGGAC
CGGCGGATGCGGCTCTCCCTCGACGTCGCCCGCAAGTTCTTCGCGCTCCAGGACATGCTCGGCTTCGACAAGGCC
AGCAAGACGGTGCAATGGCTCCTCAATACGTCAAAGGGCGCCATCAGGGAGGTCATGACTGACGAGGCGTCCTCC
GACTGCGAGGAGGACGGCTCCAGCAGCCTCTCCGTCGTCGACGGCAAGCACAAGCCGCCAGGGACGGAGGCTGAA
GGTGGTGATCACGGCGAGGGGAAGAAGCAGGTGCCAAGGGCAGCCAGAAGGGCACCCGCCAAACCAAAGCCGCAA
AGGAAATTGGCCAGTGCGCACCCGATCCCCGACAAGGAGTCGAGGACGAAGGCGCGGGAGAGGGCGAGGGAGCGG
ACTAGGGAAAAGAACCGGATGCGATGGGTGACGCTCGCGTCCACAATTAACATCGAGCCGGCAGCCACCGGCATG
GCGGCGGCGAGGGCGGACGAGTTCGTCACGAGGCCGAACAATTTGATCAGTCGCTCCTCGTCCATGAACACAGCA
AGAGCTGAATTGGAGGAGGGGTGCTCGTCGTCCATGCCGAGCGAAGCGATCATGGCTGGCTTCGGCAATGGAGGA
TACGGAAGCAGCGGTAACTACTACCAGTATCAGCTGGAGCAGCAATGGGAGCTCGGTGGAGTGGTGTTTCCCAAC
TCCCAGCCCTACTGA
>TaTB1-4B-Kronos2226-STOP (SEQ ID NO 010)
Type: DNA
Organism: Triticum turgidum spp. Durum
Length: 1065 base pairs
Definition: Mutant Allele of TaTB1-4B
Location: Mutation at the 424 base pair
ATGTTTCCTTTCTATGATTCCCCTAGCCCCATGGACTTGCCCCTTTACCAACAGCTGCAGATCAGCCCTCCCTCG
CCAAAGGCGCCGGATCACCAATCCTTGCTCTACTACCATTCCTCCCCTGCATTTGCCGCCGACTCCTTCCACCAC
AGCTACCTCTGTGCCGGTGCCGCGACGCCCACGCCGCCCGCGGCCGAGATCGACAACCAGTCGCCGCCGGAGCTG
CTGCTGATGGATCAGGCTCCGGCGCCAAGGGCAGACGGTGTTGGAACCGCACAAGGCCTGCACGGCGGTGGAGGC
CTCGACAGCGCGGCGGCCGCCGCGGCCAGGAAAGACCGGCACAGCAAGATATGCACCGCCGGCGGGATGAGGGAC
CGGCGGATGCGGCTCTCCCTCGACGTCGCCCGCAAGTTCTTCGCGCTCTAGGACATGCTCGGCTTCGACAAGGCC
AGCAAGACGGTGCAATGGCTCCTCAATACGTCAAAGGGCGCCATCAGGGAGGTCATGACTGACGAGGCGTCCTCC
GACTGCGAGGAGGACGGCTCCAGCAGCCTCTCCGTCGTCGACGGCAAGCACAAGCCGCCAGGGACGGAGGCTGAA
GGTGGTGATCACGGCGAGGGGAAGAAGCAGGTGCCAAGGGCAGCCAGAAGGGCACCCGCCAAACCAAAGCCGCAA
AGGAAATTGGCCAGTGCGCACCCGATCCCCGACAAGGAGTCGAGGACGAAGGCGCGGGAGAGGGCGAGGGAGCGG
ACTAGGGAAAAGAACCGGATGCGATGGGTGACGCTCGCGTCCACAATTAACATCGAGCCGGCAGCCACCGGCATG
GCGGCGGCGAGGGCGGACGAGTTCGTCACGAGGCCGAACAATTTGATCAGTCGCTCCTCGTCCATGAACACAGCA
AGAGCTGAATTGGAGGAGGGGTGCTCGTCGTCCATGCCGAGCGAAGCGATCATGGCTGGCTTCGGCAATGGAGGA
TACGGAAGCAGCGGTAACTACTACCAGTATCAGCTGGAGCAGCAATGGGAGCTCGGTGGAGTGGTGTTTCCCAAC
TCCCAGCCCTACTGA

### REFERENCES

The following references are referred to herein and are incorporated by reference in their entirety.
Aguilar-Martinez, J. A., Poza-Carrion, C., and Cubas, P. (2007). Arabidopsis BRANCHED1 acts as an integrator of branching signals within axillary buds. The Plant Cell 19, 458-472. https://doi.org/10.1105/tpc.106.048934
Cantrell, R. G. & Haro-Arias, E. S. (1986). Selection for spikelet fertility in a semidwarf durum wheat population. Crop Science, 26(4), 691-693. https://doi.org/10.2135/cropsci1986.0011183X00260000010x
Clarke, J. M., Marchylo, B. A., Kovacs, M. I. P., Noll, J. S., McCaig, T. N., & Howes, N. K. (1998). Breeding durum wheat for pasta quality in Canada. Euphytica 100, 163-170. https://doi.org/10.1023/A.101831363344
Cubas, P., Lauter, N., Doebley, J., & Coen, E. (1999). The TCP domain: a motif found in proteins regulating plant growth and development. The Plant Journal 18(2), 215-222. https://doi.org/10.1046/j.1365-313X.1999.00444.x
Destro, D., Miglioranza, E., Arias, C. A. A., Vendrame, J. M., & Vieira de Almeida, J. C. (2001). Main stem and tiller contribution to wheat cultivars yield under different irrigation regimes. Brazilian Archives of Biology and Technology, 44, 325-330 https://doi.org/10.1590/S1516-89122001000400001
Dixon, L. E., Greenwood, J. R., Bencivenga, S., Zhang, P., Cockram, J., Mellers, G., Ramm, K., Cavanagh, C., Swait, S. M., & Boden, S. A. (2018). TEOSINTE BRANCHED1 regulates inflorescence architecture and development in bread wheat (Triticum aestivum). The Plant Cell 30, 563-581. https://doi.org/10.1106/tpc.17.00961
Dixon, L. E., Pasquariello, M., & Boden, S. A. (2020). TEOSINTE BRANCHED1 regulates height and stem internode length in bread wheat. Journal of Experimental Botany 71(16), 4742-4750. https://doi.org/10.1093/jxb/eraa252
Doebley, J., Stec, A., & Gustus, C. (1995). Teosinte branched1 and the origin of maize: evidence of epistasis and the evolution of dominance. Genetics 141, 333-346. https:/doi.org/10.1093/genetics/141.1.333
Doebley, J., Stec, A., & Hubbard, L. (1997). The evolution of apical dominance in maize. Nature 386, 485-488. https://doi.org/10.1038/386485a0
Dong, Z., Xiao, Y., Govindarajulu, R., Feil, R., Siddoway, M. L., Nielsen, T., Lunn, J. E., Hawkins, J., Whipple, C., & Chuck, G. (2019). The regulatory landscape of a core maize domestication module controlling bud dormancy and growth repression. Nature Communications 10, 3810. https//doi.org/10.1038/s41467-019-11774-w
Duggan, B. L., Domitruk, D. R., & Fowler, D. B. (2000). Yield component variation in winter wheat grown under drought stress. Canadian Journal of Plant Science, 80(4), 739-745. https://cdnsciencepub.com/doi/10.4141/P00.006
Elhani, S., Martos, V., Rharrabti, Y., Royo, C., & Garcia del Moral, L. F. (2007). Contribution of main stem and tillers to durum wheat (Triticum turgidum L. var. durum) grain yield and its components grown in Mediterranean environments. Field Crops Research, 103, 25-35. https://doi.org/10.1016/j.fcr.2007.05.008
Fischer, R. A., Aguilar, I., & Laing, D. R. (1977). Post-anthesis sink size in a high-yielding dwarf wheat: yield response to grain number. Australian Journal of Agricultural Research, 28(2), 165-175. https://doi org/10.1071/AR9770165
Flintham, J. E., Borner, A., Worland, A. J., & Galeio, M. D. (1997). Optimizing wheat grain yield: effects of Rht (gibberellin-insensitive) dwarfing genes. Journal of Agricultural Science, 128, 11-25. https://doi.org/10.1017/S002185969003942
Garcia del Moral, L. F., Rharrabti, Y., Villegas, D., & Royo C. (2003). Evaluation of grain yield and its components in durum wheat under Mediterranean conditions: an ontogenic approach. Agronomy Journal, 95, 266-274. https://doi.org/10.2134/aggonj2003.2660
Gaudin, A. C. M., McClymont, S. A., Soliman, S. S. M., & Raizada, M. N. (2014). The effect of altered dosage of a mutant allele of Teosinte branched 1 (TB1-ref) on the root system of modern maize. BMC Genetics 15(23). https://doi.org/101.1186/1471-2156-15-23
Hubbard, L., McSteen, P., Doebley, J., & Hake, S. (2002). Expression patterns and mutant phenotype of teosinte branched1 correlate with growth suppression in maize and teosinte. Genetics 162(4), 1927-1935. https://doi.org/10.1093/genetics/162.4.1927
Krasileva, K. V., Vasquez-Gross, H. A., Howell, T., Bailey, P., Paraiso, F., Clissold, L., Simmonds, J., Ramirez-Gonzalex, R. H., Wang, X., Borril, P., Fosker, C., Ayling, S., Philips, A., Uauy, C., & Dubcovsky, J. (2017). Uncovering hidden variation in polyploid wheat. Proceedings of the National Academy of Sciences, 114(6), E913-E921. https://doi.org/10.1073/pnas.1619268114
Lewis, J. M., Mackintosh, C. A., Shin, S., Gilding, E., Kravchenko, S., Baldridge, G., Keyen, R., & Muehlbauer, G. J. (2008). Overexpression of the maize Teosinte Branched1 gene in wheat suppresses tiller development. Plant Cell Reports 27, 1217-1225. https://doi.org/10.1007/s00299-008-0543-8
Liu, J., Cheng, X., Liu, P., & Sun, J. (2017). miR156-targeted SBP-box transcription factors interact with DWARF53 to regulate TEOSINTE BRANCHED1 and BARREN STALK1 expression in bread wheat. Plant Physiology 174(3), 1931-1948. https://doi.org/10.1104/pp.17.00445
Mathews, K. L., Chapman, S. C., Trethowan, R., Singh, R. P., Crossa, J., Pfeiffer, W., van Ginkel, M., & DeLacy, I. (2006). Global adaptation of spring bread and durum wheat lines near-isogenic for major reduced height genes. Crop Science, 46, 603-613. https://doi.org/10.2125/cropsci2005.05-0056
Moayedi, A. A., Boyce, A. N., Barakbah, S. S., & Ghodsi, M. (2009). Tillering behaviors of promising durum wheat genotypes and bread wheat cultivar under different water deficit conditions. Middle Eastern and Russian Journal of Plant Science and Biotechnology, 3 (Special Issue 1), 15-19.
Motzo, R., Giunta, F., & Deidda, M. (2004). Expression of a tiller inhibitor gene in the progenies of interspecific crosses Triticum aestivum L. x T. turgidum subsp. Durum. Field Crops Research 85(1), 15-20. https://doi.org/10.1016/S0378-4290(03)00123-0
NASS-National Agricultural Statistics Service. (2019). 2019 state agriculture overview: Montana. United States Department of Agriculture. https://www.nass.gov/Quick_Stats/Ag_Overview/stateOverview.php?state=MONTANA
NASS—National Agricultural Statistics Service. (2020a). Charts and maps: All wheat acres. United States Department of Agriculture. https://www.nass.usda.gov/Charts_and_Maps/grphics/awac.pdf
NASS—National Agricultural Statistics Service. (2020b). Charts and maps: Durum wheat: production acreage by county. United States Department of Agriculture. https://www.nass.usda.gov/Charts_and_Maps/Crops_County/dw-pr.php
NASS—National Agricultural Statistics Service. (2020c). Charts and maps: Durum wheat acres. United States Department of Agriculture. https://www.nass.usda.gov/Charts_and_Maps/graphics/dwac.pdf
Naseer, A. M., Martin, J. M., Heo, H. Y., Blake, N. K., Sherman, J. D., Pumphrey, M., Kephart, K. D., Lanning, S. P, Naruoka, Y., & Talbert, L. E. (2016). Impact of a quantitative trait locus for tiller number on plasticity of agronomic traits in spring wheat. Crop Science 56(2), 595-602. https://doi.org/10.2136/cropsci2015.05.0325
Royo, C., Elias, E. M., & Manthey, F. A. (2009). Durum wheat breeding. In M. J. Carena (Ed.), Cereals-Handbook of Plant Breeding (pp. 199-226). Berlin: Springer. https://doi.org/10.1007/978-0-387-72297.9_6
Sakamoto, T. & Matsuoka, M. (2004). Generating high-yielding varieties by genetic manipulation of plant architecture. Current Opinion in Biotechnology 15 (2), 144-147. https//doi.org/10.1016/j.copbio.2004.02.003
SAS Institute Inc 2013. SAS/ACCESS® 9.4 Interface to ADABAS: Reference. Cary, NC: SAS Institute Inc.
Takeda, T., Suwa, Y., Suzuki, M., Kitano, H., Ueguchi-Tanaka, M., Ashikari, M., Matsuoka, M., & Ueguchi, C. (2003). The OsTB1 gene negatively regulates lateral branching in rice. The Plant Journal 33(3), 513-520. https://doi.org/10.10464/j.1365-313x.2003.01648.x
Tsai, H, Howell, T., Nitcher, R., Missirian, V., Watson, B., Ngo, K. J., Lieberman, M., Fass, J., Uauy, C., Tran, R. K., Khan, A. A., Filkov, V., Tai, T. H., Dobcovsky, J., & Comai, L. (2011). Discovery of rare mutations in populations, TILLING by sequencing. Plant Physiology, 156, 1257-1268. https://doi.org/10.1104/pp.110.169748
Xu, K., Wang, Y., Shi, L., Sun, F., Liu, S., & Xi, Y. (2016). PvTB1, a Teosinte Branched1 gene homolog, negatively regulates tillering in switchgrass. Journal of Plant Growth Regulation 35:44-53. https://doi.org/10.1007/s00344-015-9505-x
Addisu, M., Snape, J. W., Simmonds, J. R., & Gooding, M. J. (2009). Reduced height (Rht) and photoperiod insensitivity (Ppd) allele associations with establishment and early growth of wheat in contrasting production systems. Euphytica, 166, 249-263. https://doi.org/10.1007/s10681-008-9838-7
Amram, A., Fadida-Myers, A., Golan, G., Nashef, K., Ben-David, R., & Peleg, Z. (2015). Effect of GA-sensitivity on wheat early vigor and yield components under deep sowing. Frontiers in Plant Science, 6, 487. https//doi.org/10.3389/2015.00487
Bai, M. Y., Shang, J. X., Oh, E., Fan, M., Bai, Y., Zentella, R., Sun, T. P., & Wang Z. Y. (2012). Brassinosteroid, gibberellin and phytochrome impinge on a common transcription module in Arabidopsis. Nature Cell Biology, 14, 810-817. https://doi.org/10.1038/ncb2546
Bates, D., Mächler, M., Bolker, B., & Walker, S. (2015). Fitting linear mixed-effects models using Ime4. Journal of Statistical Software, 67(1), 1-48. https://doi.org/10.18637/jss.v067.101
Cantrell, R. G. & Haro-Arias, E. S. (1986). Selection for spikelet fertility in a semidwarf durum wheat population. Crop Science, 26(4), 691-693. https://doi.org/10.2135/cropsci1986.0011183X002600040010x
Chandler, P. M., Marion-Poll, A., Ellis, M., & Gubler, F. (2002). Mutants at the Slender1 locus of barley cv Himalaya molecular and physiological characterization. Plant Physiology 129, 181-190. https://doi.org/10.1104/pp.010917
Chandler, P. M., Harding, C. A., Ashton, A. R., Mucair, M. D., Dixon, N. E., & Mander, L. N. (2008). Characterization of gibberellin receptor mutants of barley (Hordeum vulgare L.) Molecular Plant, 1(2), 285-294. https://doi.org/10.1093/mp/ssn002
Clarke, J. M., Marchylo, B. A., Kovacs, M. I. P., Noll, J. S., McCaig, T. N., & Howes, N. K. (1998). Breeding durum wheat for pasta quality in Canada. Euphytica 100, 163-170. https://doi.org/10.1094/CCHEM-05-17-0103-FI
Elias, E. M. & F. A. Manthey. 2007. Registration of ‘Divide’ durum wheat. Journal of Plant Registration. 1: https://doi.org/10.3198/jpr2006.05.0281crc
Feiz, L., Beecher, B. S., Martin, J. M., and Giroux, M. J. (2009). In planta mutagenesis determines the functional regions of the wheat puroindoline proteins. Genetics, 183(3), 853-860. https://doi.org/10.1634/genetics.109.106013
Fick, G. N. & Qualset, C. O. (1976). Seedling emergence, coleoptile length, and plant height relationships in crosses of dwarf and standard-height wheats. Euphytica, 25, 679-684. https://doi.org/10.1007/BF00041606
Gale, M. D. & Marshall, G. A. (1973). Insensitivity to gibberellin in dwarf wheats. Annals of Botany, 37, 729-735. https://doi.org/10.1093/oxfordjournals.aob.a084741
Gale, M. D. & Youssefian, S. (1985). Dwarfing genes in wheat. In Russell, G. E. (Ed.); Progress in Plant Breeding (pp. 1-35). Butterworths, London.
Hedden, P. (2003). The genes of the Green Revolution. TRENDS in Genetics, 19(1), 5-9. https://doi.org/10.1016/s09625(02)00009-4
Hogg, A. C., Carr, P. M., Eberly, J. O., Chen, C., Kowatch, C., Crutcher, F. K., Lamb, P. F., McNamara, K., Haney, E., Kephart, K. D., Smith, V., Dykes, L., Chen, X., Huang, L., & Giroux, M. J. (2021) Registration of Lustre durum wheat. Journal of Plant Registrations, in review, June 2021.
Hoogendoorn, J., Rickson, J. M., & Gale, M. D. (1990). Differences in leaf and stem anatomy related to plant height of tall and dwarf wheat (Triticum aestivum L.). Journal of Plant Physiology, 136, 72-77. https://doi.org/10.1016/S0176-1617(11)81618-4
Ikeda, A., Ueguchi-Tanaka, M., Sonoda, Y., Kitano, H., Koshioka, M., Futsuhar, Y., Matsuoka, M., & Yamaguchi, J. (2001). Slender rice, a constitutive gibberellin response mutant, is caused by a null mutation of the SLR1 gene, an orthologs of the height-reducing gene GAI/RGA/RHT/D8. The Plant Cell, 13, 999-1010. https://doi.org/10.1105/tpc.13.5.999
Jobson, E. M., Martin, J. M., Schneider, T. M., & Giroux, M. J. (2018). The impact of the Rht-B1b, Rht-D1b, and Rht-8 wheat semi-dwarfing genes on flour milling, baking, and micronutrients. Cereal Chemistry, 95, 770-778. https://doi.org/10.1002/cche.10091
Jobson, E. M., Johnston, R. E., Oiestad, A. J., Martin, J. M., & Giroux, M. J. (2019). The impact of the wheat Rht-B1b semi-dwarfing allele on photosynthesis and seed development under field conditions. Frontiers in Plant Science, 10, (51). https//doi.org/10.3389/fpis.2019.00051
Jobson, E. M., Martin, J. M., Sharrock, R., Hogg, A. C., & Giroux, M. J. (2020). Identification and molcular characterizations of novel Rht-1 alleles in hard red spring wheat. Crop Science, Accepted Article, https://doi.org/10.1002/csc2.20375 (in press).
Knopf, C., Becker, H., Ebmeyer, E., & Korzun, V. (2008). Occurrence of three dwarfing Rht genes in German winter wheat varieties. Cereal Research Communications, 36(4), 553-560. https://doi.org/10.1556/CRC36.2008.4.4
Li, A., Yang, W., Lou, X., Liu, D., Sun, J., Guo, X., Wang, J., Li, Y., Zhan, K., Ling, H. Q., & Zhang, A. (2013). Novel natural allelic variations at the Rht-1 loci in wheat. Journal of Integrative Plant Biology, 55, 1026-1037. https://doi.org/10.1111/jipb.12103
Li, X. P. & Lan, S. (2006). Effects of different Rht-B1b, Rht-D1b, and Rht-B1c dwarfing genes on agronomic characteristics in wheat. Cereal Research Communications, 34(2-3), 919-924. https://doi.org/10.1556/CRC.34.2006.2-3.220
Liatukas, Z. & Ruzgas, V. (2011). Coleoptile length and plant height of modern tall and semi-dwarf European winter wheat varieties. Acta Societatis Botanicorum Poloniae, 80, 197-203. https://doi.org/10.5586.aspb.2011.018
Mo, Y., Pearce, S., & Dubcovsky, J. (2018). Phenotypic and transcriptomic characterization of a wheat tall mutant carrying an induced mutation in the C-terminal PFYRE motif of RHT-B1b. BMC Plant Biology, 18(1). https://doi.org/10.1186/s12870-012-1465-4
Moayedi, A. A., Boyce, A. N., Barakbah, S. S., & Ghodsi, M. (2009). Tillering behaviors of promising durum wheat genotypes and bread wheat cultivar under different water deficit conditions. Middle Eastern and Russian Journal of Plant Science and Biotechnology, 3 (Special Issue 1), 15-19.
Pandey, M., Singh, A. K., DePauw, R. M., Bokore, F. E., Ellouze, W., Knox, R. E. & Cuthbert, R. D. 2015. Coleoptile length, gibberellin sensitivity, and plant height variation of durum wheat in Canada. Canadian Journal of Plant Science. 95: 1259-1264. https://doi.org/10.4141/cjps-2015-154
Pearce, S., Saville, R., Vaughan, S. P., Chandler, P. M., Wilhelm, E. P., Sparks, C. A., Al-Kaff, N., Korolev, A., Boulton, M. I., Phillips, A. L., Hedden, P., Nicholson, P., & Thomas, S. G. (2011). Molecular characterization of Rht-1 dwarfing genes in hexaploid wheat. Plant Physiology, 157, 1820-1831. https://doi.org/10.1104/pp.111.183657
Peng, J., Carol, P., Richards, D. E., King, K. E., Cowling, R. J., Murphy, G. P., & Harberd, N. P. (1997). The Arabidopsis GAI gene defines a signaling pathway that negatively regulates gibberellin responses. Genes and Development, 11, 3194-3205. https.//doi.org/10.1101/gad.11.23.2194
Peng, J., Richards, D. E., Hartley, N. M., Murphy, G. P., Devos, K. M., Flintham, J. E., Beales, J., Fish, L. J., Worland, A. J., Pelica, F., Sudhakar, D., Christou, P., Snape, J. W., Gale, M. D., & Harberd, N. P. (1999). ‘Green revolution’ genes encode mutant gibberellin response modulators. Nature, 400, 256-261. https://doi.org/10.1038/22307
Schillinger, W. F., Donaldson, E., Allan, R. E., & Jones, S. S. (1998). Winter wheat seedling emergence from deep sowing depths. Agronomy Journal, 90, 582-586. https://doi.org/10.2134/agronj1998.000219620000050002x
Slade, A., Fuerstenberg, S., Loeffler, D., Steine, M. N., & Facciotti, D. (2005). A reverse genetic, nontransgenic approach to wheat crop improvement by TILLING. Nature Biotechnology, 23, 75-81. https://doi.org/10.1038/nbt1043
Troccoli, A., Borrelli, G. M., De Vita, P., Fares, C., & Di Fonzo, N. (2000). Durum wheat quality: A multidisciplinary concept. Journal of Cereal Science 32, 99-113. https://doi.org/10.1006/jcrs.2000.0322
Van De Velde, K., Thomas, S. G., Heyse, F., Kaspar, R., Van Der Straeten, D., & Rohde, A. (2021) N-terminal truncated RHT-1 proteins generated by translational reinitiation cause semi-dwarfing of wheat Green Revolution alleles. Molecular Plant, 14, 679-687. https://doi.org/10.1016/j.molp.2021.01.002
Wilhelm, E. P., Howells, R. M., Al-Kaff, N., Jia, J., Baker, C., Leverington-Waite, M. A., Griffiths, S., Greenland, A. J., Boulton, M. I., & Powell, W. (2013). Genetic characterization and mapping of the Rht-1 homoeologs and flanking sequences in wheat. Theoretical and Applied Genetics, 126, 1321-1336. https://doi.org/10.1007/00122-013-2055-3
Winkler, R. G. & Freeling, M. (1994). Physiological genetics of the dominant gibberellin-nonresponsive maize dwarfs, Dwarf8 and Dwarf9. Planta, 193, 341-348. https://doi.org/10.1007/BF00201811
Worland, A. J., Korzun, V., Roder, M. S., Ganal, M. W., & Law, C. N. (1998). Genetic analysis of the dwarfing gene Rht8 in wheat. Part II. The distribution and adaptive significance of allelic variants at the Rht8 locus of wheat as revealed by microsatellite screening. Theoretical and Applied Genetics, 96, 1110-1120. https://doi.org/10.1007/s001220060846
Youssefian, S., Kirby, E. J. M., & Gale, M. D. (1992). Pleiotropic effects of the GA-insensitive Rht dwarfing genes in wheat. 1. Effects on development of the ear, stem and leaves. Field Crops Research, 28, 179-190. https://doi.org/10.1016/0378-4290(92)90040-G

Thus, specific embodiments, methods of optimization of small grain cereal forage and grain production via mutation of the TB1 and RHT genes have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification and claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A method of producing a mutated wheat plant having an increased biomass content, comprising: introducing at least one TB1 null allele mutation to a wheat plant seed to produce a first TB1 mutated wheat plant seed.

2. The method of claim 1, further comprising introducing a second TB1 null allele mutation to a wheat plant seed to produce a second TB1 mutated wheat plant seed, crossing the second TB1 mutated wheat plant seed with the first TB1 mutated wheat plant seed, and producing a third TB1 mutated wheat plant seed.

3. The method of claim 1, where the biomass content of the mutated wheat plant is increased at least 5% as compared to the biomass content of a non-mutated wheat plant.

4. The method of claim 1, where the biomass content of the mutated wheat plant is increased at least 10% as compared to the biomass content of a non-mutated wheat plant.

5. The method of claim 1, where the biomass content of the mutated wheat plant is increased at least 15% as compared to the biomass content of a non-mutated wheat plant.

6. The method of claim 1, wherein the at least one TB1 null allele mutation is found in a genome in the group consisting of SEQ ID NO. 007, 009, and 010.

7. A mutated wheat plant produced from the method of claim 1.

8. The mutated wheat plant of claim 7, wherein the plant is winter wheat or spring wheat.

9. The mutated wheat plant of claim 7, wherein the plant is durum wheat, hexaploid wheat, or triticale wheat.

10. A mutated wheat plant seed produced from the method of claim 1.

11. The mutated wheat plant seed of claim 10, wherein the seed is winter wheat or spring wheat.

12. The mutated wheat plant seed of claim 10, wherein the seed is durum wheat, hexaploid wheat, or triticale wheat.

13. A method of producing a mutated wheat plant having an increased seed protein content, comprising: introducing at least one TB1 null allele mutation to a wheat plant seed to produce a first TB1 mutated wheat plant seed.

14. The method of claim 13, further comprising introducing a second TB1 null allele mutation to a wheat plant seed to produce a second TB1 mutated wheat plant seed, crossing the second TB1 mutated wheat plant seed with the first TB1 mutated wheat plant seed, and producing a third TB1 mutated wheat plant seed.

15. The method of claim 13, where the seed protein content of the mutated wheat plant is increased at least 1% as compared to the seed protein content of a non-mutated wheat plant.

16. The method of claim 13, where the seed protein content of the mutated wheat plant is increased at least 3% as compared to the seed protein content of a nonmutated wheat plant.

17. The method of claim 13, where the seed protein content of the mutated wheat plant is increased at least 5% as compared to the seed protein content of a nonmutated wheat plant.

18. The method of claim 13, wherein the at least one TB1 null allele mutation is found in a genome in the group consisting of SEQ ID NO. 007, 009, and 010.

19. A mutated wheat plant produced from the method of claim 13.

20. The mutated wheat plant of claim 19, wherein the plant is winter wheat or spring wheat.

21. The mutated wheat plant of claim 19, wherein the plant is durum wheat, hexaploid wheat, or triticale wheat.

22. A mutated wheat plant seed produced from the method of claim 13.

23. The mutated wheat plant seed of claim 22, wherein the seed is winter wheat or spring wheat.

24. The mutated wheat plant seed of claim 22, wherein the seed is durum wheat, hexaploid wheat, or triticale wheat.

25-57. (canceled)
